SEQUENCE LISTING (83 sequences)

SEQ ID NO: 1 - DNA, 8905 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatccgtgatagtaacctgatagtaacctgataatagcagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 2 - DNA, 12082 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccgctagctgataatagccgataccctgtcaccggatgtgctttccggtctgatgagtccgtgaggacgaaacaggactgtcgcagagacaaccgccgataccctgtcaccggatgtgctttccggtctgatgagtccgtgaggacgaaacaggactgtcgcagggcaccgtatcaactctgagatgcaggtacatccagctgatgagtcccaaataggacgaaacgcgcttcggtgcgtcctggattccactgctatccactattcatccaaagagactatcaactctgagatgcaggtacatccagctgatgagtcccaaataggacgaaacgcgcttcggtgcgtcctggattccactgctatccactattcatccgtctccgatcgtctagagcctgacctagctgacctagccgccaccatgcagaggctgcaggtagtgctgggccacctgaggggtccggccgattccggctggatgccgcaggcagcgccttgcctgagatctactagtggtgacctagctgacctagccgtcaccatggaccctgttgtgctgcaaaggagagactgggagaaccctggagtcacccagctcactgaacttaaccctagccctgccaccatggcttggaggaactccgaggaagccaggactgacagtaacagtaggcagcgccgccatggcaacggagagtggaggtttgcctggtttgacgctaaggatagtgtggccaccatggggctggagtgcgacctcccagaggcggctgacgttaaagttagcagcagccgccatgggcacggctacgacgcgcccatctacagtgacgttagcactaagatcgccaccatggccccttttgtgcccaccgagaacccgactaactgtagcagtgacacctgccgccatggcgagagctggctgcaagaaggacagactaagattagctgtgacggagccaccatggccttccacctctggtgcaacggcaggtgtaacggtagcggtgaagacagccgccatggctccgagttcgacctctctgccttccgtaacgctgaagataacagggccaccatggtggtgctcaggtggtccgacggcagctatagggctgaccgtaacatgtgccgccatggtggcatcttcagggacgtcagcctgcttagcactgagactaaccaggccaccatggtccacgttgccacgaggttcaacgacgatagcagtgaagctaagctgggccgccatgggcagatgtgtggagaactcagagagtctgacagtagcactaagagcgccaccatgggcgagacccaggtggcctctggcacagctgactttagagctaagatcagccgccatggaggaggctacgccgacagagccacccttgagcttagcgttaagaacgccaccatggggtctgccgagacccccaacctctacagtaacgttagggctgagcacagccgccatggcacgctcatcgaagccgaagcctgcgataacggtgacagtagagtcgccaccatggacggcctgctgctgctcaacggcaagcctaagcttgacagtagagtcagccgccatgggcaccatcctctgcacggacaagtcattaaggctgagactagggtggccaccatggtgctcacgaagcagaacaacttcaacgctaagagtgactctagctaccgccgccatggtctctggtacaccctgtgcgacaggaataaccttagggttgacgaggccaccatggtcgagacacacggcatggtgcccacgaataagcttagagctgaccccagccgccatggtgccatgtccgagagagtcaccaggattaagcatagagctgagaacgccaccatggtcatcatctggtctctgggcaacgagtctaagcatagagctgaccacggccgccatggcaggtggatcaagtctgccgaccccagtagacctgagcataacgaagccaccatggcagacaccacagccacagacatcatctgtagcattgaggctaaggtcggccgccatgggcccttccctgctgtgcccaagtggagtagcactgagtgtaactctgccaccatggaaacgagacctctcatcctgtgcgagtatagacctgaaagtaaacccggccgccatggctttgccaagtactggcaagccttcactgagtatagcactaagcaagccaccatggcccgcgactgggtggaccagtcactcattgagtatagcgataacggcagccgccatggtgcctacggaggagactttggcgacactgagaatagcactaagttcgccaccatgggcctggtctttgccgaccggactccgcctgaccctagcactaaggccagccgccatggacagttcttcccgttcacgctgtctggtgaaactaacgatagcacagccaccatggtcttcagacactccgacaacgagctgcttgactgtaaggctaggctgggccgccatggtctggcttctggcgaggtgcctctggctgaggctaatcatagaaaggccaccatggaactgcccgagctgcctcagccagagtctgacgataaccttaggctcagccgccatggggttcagcccaacgcaacagcttggtctgagcctagccataactctgccaccatggagtggaggctggccgagaacctctcggttgaccttagggctaactctcgccgccatggtcacctcacaacatccgaaatggagtttgacattaggcttaacaacgccaccatggagttcaacaggcagtctggcttcctgtctgagattaggactaaagacagccgccatggcctctctcctctccgagacctgttcactagggctgagcttaacactgccaccatggtgtcagaggccaccaggatcgacccaaatagttgtgaggataagtggagccgccatggacactaccaggccgaggctgccctgcttagctgtgaagctaaccagcgaatgcctggggctctcatcaccacagcccacgcttgtagccctgaagctaagacagcgaatgcctggggcaagacctacagaatcgacggccataggcctgaggctaaccagcgaatgcctggggctgcctccgacacacctcaccctgctaggattgagcttaactcagcgaatgcctggggcgcagagagggtcaactggctgggtgagggtaatcataggcagcgaatgcctggggccacagctgcctgcttcgacacctgtgagcttaaccttagcgcagcgaatgcctggggcgtgttcccttccgagaacggccttgagtgtaacactaggcagcgaatgcctggggctcaccagtggaggggagacttgcctgacagtaactctaggccagcgaatgcctggggcatggaaacctctcacagacagcttgaccataaggataggcagcgaatgcctggggccgacggcttccacatgggcattggtgaagataactctagctcagcgaatgcctggggcgagttcccgacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 3 - DNA, 9536 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatccgtgatagtaacctgatagtaacctgataatagcagatctgcagcttggggtatcagtcacattcggctggtacccctccggaagcgaatgggagccgacgatgtggtcgattcttcgaaatcttttgtcatggaaaacttttcttcgtaccacgggacgaaacctggttatgtggattccattcaaaaaggtaggtttaatgttcgttagatatagttgcagcttctaacaaacatcaaaactgattatgcttagggtttttctttttattttttaacaggcatacaaaagccaaaatctggtacacaaggaaactacgacgacgattggaaaggtgaggcactcagggtgcaggacttggactataaacccaatggagaagatagcccttcaacctctgtgacttttctaaagctactttcccccctttttgccttagggttttacagtaccgacaacaaatacgacgctgcgggatactctgtggacaacgaaaacccgctctctggaaaagctggaggcgtggtcaaagtcacgtatccaggtcaaaggaaataaatttttagaatccatttatttgtactgaagtaaaagttcacatatgcaacttctatttaataggttaacttcacaaacctattctgtaccatagggctcacgaaagttctcgcactcaaagtggacaatgccgaaactatcaagaaagagttgggtctctctctcaccgaaccgctcatggagcaagtcggaacggaagagtttatcaaaagattcggcgatggtgcttcgcgtgtcgtgctcagccttcccttcgccgaggggagttccagcgtcgagtacatcaacaactgggaacaggtatgaatgcaattgttggcatctttttttaaagttatgtttaagatatgaagttaaaattattttcaaatctgtagttaggctagtcattaaaactttttccaggtcagaacttacgacctgcttttatttccaaataggcgaaagcgctcagcgtcgaactcgagatcaacttcgaaacccgtggaaaacgtggccaagatgcgatgtacgagtatatggctcaagcctgtgcaggtgggcagctcatgagcccaggagattctgtcttgtttctgtgcctagtggagtttgttagtttgctgtgattagctggcaacggaaactggattcatgttgcagagggtttttctcatctgggtattcttggttttccacttacactttccccgtcttttctgtaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 4 - DNA, 9174 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccggtaccatgggagccgacgatgtggtcgattcttcgaaatcttttgtcatggaaaacttttcttcgtaccacgggacgaaacctggttatgtggattccattcaaaaaggcatacaaaagccaaaatctggtacacaaggaaactacgacgacgattggaaagggttttacagtaccgacaacaaatacgacgctgcgggatactctgtggacaacgaaaacccgctctctggaaaagctggaggcgtggtcaaagtcacgtatccaggtgagtctctagccctgcctttgcctgtcctctcagcacttccattagccagctacctacttccatccactcccaaacttcagggctctgcctgcccccagaggcacaggacttagttctgggaccagggatcaggccgcagccctggcctgctgttgcttctgtcagggacttgcctttgaccccagcctctctgaccctcagggtctccttggggagctcttctgaatttgggctggcagataccccacccagaccaggtctgccggtgcggcagggccagtggggcaggttggctgtggctgctgtgccctagtctgccctttctgacttgcagggctcacgaaggttctcgcactcaaggtggacaatgccgaaactatcaagaaagagttgggtctcagcctcaccgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 5 - DNA, 10034 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccctggctgagctgtacaagggtaagtcactgactgtctatgcctgggaaagggtgggcaggagatggggcagtgcaggaaaagtggcactatgaacccaactacacaaatcagcgatttcaacaacaactacacaaatcagcgatttcaattgtactaaccttcttctctttcctctcctgacaggaggagccatcatcgcccgatatcccaatcgcttaccgattcagaatctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcaatcgcttaccgattcagaatccctacggcaagctgaccctgaagttcatcagatctgcagcttggggtatcagtcacattcggctggtacccctccggaagcgaatgggagccgacgatgtggtcgattcttcgaaatcttttgtcatggaaaacttttcttcgtaccacgggacgaaacctggttatgtggattccattcaaaaaggtaggtttaatgttcgttagatatagttgcagcttctaacaaacatcaaaactgattatgcttagggtttttctttttattttttaacaggcatacaaaagccaaaatctggtacacaaggaaactacgacgacgattggaaaggtgaggcactcagggtgcaggacttggactataaacccaatggagaagatagcccttcaacctctgtgacttttctaaagctactttcccccctttttgccttagggttttacagtaccgacaacaaatacgacgctgcgggatactctgtggacaacgaaaacccgctctctggaaaagctggaggcgtggtcaaagtcacgtatccaggtcaaaggaaataaatttttagaatccatttatttgtactgaagtaaaagttcacatatgcaacttctatttaataggttaacttcacaaacctattctgtaccatagggctcacgaaagttctcgcactcaaagtggacaatgccgaaactatcaagaaagagttgggtctctctctcaccgaaccgctcatggagcaagtcggaacggaagagtttatcaaaagattcggcgatggtgcttcgcgtgtcgtgctcagccttcccttcgccgaggggagttccagcgtcgagtacatcaacaactgggaacaggtatgaatgcaattgttggcatctttttttaaagttatgtttaagatatgaagttaaaattattttcaaatctgtagttaggctagtcattaaaactttttccaggtcagaacttacgacctgcttttatttccaaataggcgaaagcgctcagcgtcgaactcgagatcaacttcgaaacccgtggaaaacgtggccaagatgcgatgtacgagtatatggctcaagcctgtgcaggtgggcagctcatgagcccaggagattctgtcttgtttctgtgcctagtggagtttgttagtttgctgtgattagctggcaacggaaactggattcatgttgcagagggtttttctcatctgggtattcttggttttccacttacactttccccgtcttttctgtaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 6 - DNA, 9441 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgtctacggcaagctgaccctgaagttcatccaaaactacacaaatcagcgatttcaacaaaactacacaaatcagcgatttcaacaacaaaactacacaaatcagcgatttcaacaaaatcgcttaccgattcagaatcgcccggggatctgtccactgctgttgctgttttgggcatccatcaggagaaggctcacggcaacaaagtgctcggtgcctttactacgggggggggggggggggggggggggccgaagttgtcagcccagaaccccacacgagttttgccactgggaagctgtgatccagtgcaggctgggacagccgacctccagcgcgcggtcaccggtaccatgggagccgacgatgtggtcgattcttcgaaatcttttgtcatggaaaacttttcttcgtaccacgggacgaaacctggttatgtggattccattcaaaaaggcatacaaaagccaaaatctggtacacaaggaaactacgacgacgattggaaagggttttacagtaccgacaacaaatacgacgctgcgggatactctgtggacaacgaaaacccgctctctggaaaagctggaggcgtggtcaaagtcacgtatccaggtgagtctctagccctgcctttgcctgtcctctcagcacttccattagccagctacctacttccatccactcccaaacttcagggctctgcctgcccccagaggcacaggacttagttctgggaccagggatcaggccgcagccctggcctgctgttgcttctgtcagggacttgcctttgaccccagcctctctgaccctcagggtctccttggggagctcttctgaatttgggctggcagataccccacccagaccaggtctgccggtgcggcagggccagtggggcaggttggctgtggctgctgtgccctagtctgccctttctgacttgcagggctcacgaaggttctcgcactcaaggtggacaatgccgaaactatcaagaaagagttgggtctcagcctcaccgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 7 - DNA, 9635 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagacgcggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcgtgcgcacctacggcaagctgaccctgaagttcatccaacaaaactacacaaatcagcgatttccacaacaactacggcaagctgaccctgaagttcatccaacaaaactacacaaatcagcgatttccacaacaactacggcaagctgaccctgaagttcatccaacaaaactacacaaatcagcgatttccagcaaggcaaccaaaggctctttttagagccacctttcaacgcgcaaggcaacaaaaggcccttttcagggccacctttcaagagggcgcaaggcaaccaaaggctcttttcagagccacctttcaaggcgcaaggcaaccaaaggctcttttcagagccccctttattggacaaactacctacagagatttaaagctctaaggtaaatataaaatttttaagtgtataatgtgttaaactactgattctaattgtttgtgtattttagattccaacctatggaactgatcaatcggagcagtggtggaatccctttaaacatttgcgtctgacacaactgtgttcactagcaacctcaaacagacaccacggtgcatctgactcctgaggagaagtctgccgttactgccctgtggggcaaggtgaacgtggacgaagttggtgctgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtggtctacccttggacccagaggttctttgagtcctttggggatctgtccactgctgaagctgttacgggcaaccctaagctgaaggctcctggcaagaaagtgctcggtgcctttagtgatcgcctggctcacctggacaacctcaagggcacctttgccacgctgagtgagctgcactgtgacaagctgcacgtgtatcctgagaacttcaggctcctgggcaacgtgctggtctgtgtgctggcccatcaccttggcaaagaattcaccccaccagtgcaggctgcctatcagaaagtggtggctggtgtggctaacgccctggcccacaagtatcactaagctcgctttcttgctgtccgatttctattagaggttcctttgttccctaagtccaactacgaaactgggggatattctgaagggccttgagcatctggattctgcctggcgcgccggtcaccccggatccgtgatagtaacctgatagtaacctgataatagcagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 8 - DNA, 8866 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 9 - DNA, 9665 bp, Artificial Sequence
  Synthetic
agatctgcgcagcaccatggcctgaaataacctctgaaagaggaacttggttaggtacctaccggaaggaacccgcgctatgacggcaataaaaagacagaataaaacgcacggtgttcttataatggttacaaataaagcaatagcatcacaaatttcacaaataaagcatttttttcactgcattctagttgtggtaataaaatatctttattttcattacatctgtgtgttggttttttgtgtgtggcctcccaaagtgctgggattacaggcatgagccatcgagcccaacccaattttttttttttttaattttactttctgcaatcattcatccattcagccagtgcggtatttctgaggtgtgttcgatcgcggatccatgcctgccgcagtacagttgtgagccaaatgagactgagactagttcccgccctccaagagcttgcaagacccgcagtggcgtaaaaacactaacatcttttagtgatcgattctgcactccaggggttttcaatctactacaagagtgaataagagttcgcctttgtctgatatctgttgtcattctctctcgcttctttaactgattttttctcagctaataaaacatccacccacaaccccccgaacgcccgcaaacaccaggccactctagcaaaacctctctcactccgcctgcgcaatccagctgacttccggttacagataaccacgtgattgggaacccttgctgcgcatgtctagtaggaagtcggactataccactttccctacggaaggggtacttttttatgtttttaagtttaaaaccgatttctgatatttgacttttatcatttcaggcctatatggaggctatgagtgagtttagtgtggcagaagatgaaagaaccggacaggaatacggacgaaattggagcagggtttgggctctccccttcgcagataatcggaggagccgggcccgagcgagctctttcctttcgctgctgcggccgcagccgtgaggtgagggcgagctggtctccatcaggcgctgacgcgtgtcgacaagggactgtcggtcttgggaccgcagctggggttgggggagatgaaatggaggccgccctaaagcggccggtcccggggtttggggtaggccggagcactttcgtcccgggcctccggagtgagggggggcggggagcgtcgcagcaactgagaccaggaaaagtctgccccggctggtgccgcaccgcacacgtgtccggtcgacccacgcgagcagagcaaacggagcgaacaagaccaagccgtgggccctttcttgcttggcacacccggagcggagccgatctctgctttcacgtgatgtagggcaagcctagtgtaggccccaggcctccgactgccgagagaggtgatctctaactcttgactccattcactcctttggcctctcataaaggaaatctctgcgaatagccgaacgaggcttgttactgtgataaaacagggaaataagcccagaaaacagagtaacttgcctgcattcctagactagaaatcaggtctactcacctcgaatattctttaaacgctgagtaccagaaatggcataacccccctattcaatccaataagtccttggcttgactttccagaggagaaatgcgaacatgaggctccgagaggtgaaggcatagcgtgggttttgaagtcttaaacccaagggggccagctgcatagcccagagccttaaagatgatttagggaagagtcttatttcgcggctgtggtgtgggtcacaaagggcaggtcttgatggggacgttcattcttgcccaggattggctttcagagtctaatcatgttttctgtgtgtctagtatcctcaggcttcagaagaggctcgcctctagtgtcctccgctgtggcaagaagaaggtctggaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggtcccacgcgtcaattggaaaacttacgctgagtacttcgatctccctacggcaagctgaccctgaagttcaacagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 10 - DNA, 8290 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgacgagctcaagcttcccggcgcgccccggtccgtccggactacggcaagctgaccctgaagttcatcccaaaacttacgctgagtacttcgatctggtcaccccggatccgtgatagtaacctgatagtaacctgataatagcagatctgcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 11 - DNA, 6441 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtacccccaagcttaccggtcccacgcgtcaattggaaaacttacgctgagtacttcgatctccctacggcaagctgaccctgaagttcaacagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 12 - DNA, 6368 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatctgctagcggcgcgcccccggtacctgataagcctagcagcgaatgcctggggcagacgatgtcgtcgacagtagcaagagctttgtgatggagaattttagtagctatcatggtactaagccgggatacgtagatagtatccagaagggaatccagaaacccaagagcggaactcagggcaactacgatgacgactggaagggtttctactcgaccgataacaaatatgatgcagccggttacagcgtggacaacgagaatcctttgagcggcaaggcaggcggggtcgtcaaggtcacctaccccggtttaaccaaagtgttagctttgaaggtggacaacgcggagacaatcaaaaaggaactcggactctcgctcacggagcctcttatggaacaggtcggcaccgaggaattcataaagcgttttggagatggagcaagtagggttgtcttatcattgccatttgcggaaggctcgagctcagtggagtacataaacaattgggagcaagccaaggcactctcagttgagctggagatcaacttcgagacaagaggcaagagagggcaggacgcgatgtacgagtacatggcacaggcgtgcgctggcaacagagtccgtaggtgaacataagcataggcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 13 - DNA, 6015 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagacgcggtacctagctaggtagcaattgaccggtcccacgcgtcaattggaaaacttacgctgagtacttcgatctccctacggcaagctgaccctgaagttcaacagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 14 - DNA, 6352 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatcttgtacattcgaacgccgccatgggcgctgatgatgttgttgattcttctaaatcttttgtcatggaaaacttttcttcgtaccacgggactaaacctggttatgtggattccattcaaaaaggtatacaaaagccaaaatctggtacacaaggaaattatgacgatgattggaaagggttttatagtaccgacaataaatacgacgctgcgggatactctgtggataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatagtgaactagttccggatctagagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 15 - DNA, 6658 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccatgggagccgacgatgtggtcgattcttcgaaatcttttgtcatggaaaacttttcttcgtaccacgggacgaaacctggttatgtggattccattcaaaaaggcatacaaaagccaaaatctggtacacaaggaaactacgacgacgattggaaagggttttacagtaccgacaacaaatacgacgctgcgggatactctgtggacaacgaaaacccgctctctggaaaagctggaggcgtggtcaaagtcacgtatccaggtgagtctctagccctgcctttgcctgtcctctcagcacttccattagccagctacctacttccatccactcccaaacttcagggctctgcctgcccccagaggcacaggacttagttctgggaccagggatcaggccgcagccctggcctgctgttgcttctgtcagggacttgcctttgaccccagcctctctgaccctcagggtctccttggggagctcttctgaatttgggctggcagataccccacccagaccaggtctgccggtgcggcagggccagtggggcaggttggctgtggctgctgtgccctagtctgccctttctgacttgcagggctcacgaaggttctcgcactcaaggtggacaatgccgaaactatcaagaaagagttgggtctcagcctcaccgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 16 - DNA, 6964 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatccggtaccgaggagatctgccgccgcgatcgccggaagcgaatgggagccgacgatgtggtcgattcttcgaaatcttttgtcatggaaaacttttcttcgtaccacgggacgaaacctggttatgtggattccattcaaaaaggtaggtttaatgttcgttagatatagttgcagcttctaacaaacatcaaaactgattatgcttagggtttttctttttattttttaacaggcatacaaaagccaaaatctggtacacaaggaaactacgacgacgattggaaaggtgaggcactcagggtgcaggacttggactataaacccaatggagaagatagcccttcaacctctgtgacttttctaaagctactttcccccctttttgccttagggttttacagtaccgacaacaaatacgacgctgcgggatactctgtggacaacgaaaacccgctctctggaaaagctggaggcgtggtcaaagtcacgtatccaggtcaaaggaaataaatttttagaatccatttatttgtactgaagtaaaagttcacatatgcaacttctatttaataggttaacttcacaaacctattctgtaccatagggctcacgaaagttctcgcactcaaagtggacaatgccgaaactatcaagaaagagttgggtctctctctcaccgaaccgctcatggagcaagtcggaacggaagagtttatcaaaagattcggcgatggtgcttcgcgtgtcgtgctcagccttcccttcgccgaggggagttccagcgtcgagtacatcaacaactgggaacaggtatgaatgcaattgttggcatctttttttaaagttatgtttaagatatgaagttaaaattattttcaaatctgtagttaggctagtcattaaaactttttccaggtcagaacttacgacctgcttttatttccaaataggcgaaagcgctcagcgtcgaactcgagatcaacttcgaaacccgtggaaaacgtggccaagatgcgatgtacgagtatatggctcaagcctgtgcaggtgggcagctcatgagcccaggagattctgtcttgtttctgtgcctagtggagtttgttagtttgctgtgattagctggcaacggaaactggattcatgttgcagagggtttttctcatctgggtattcttggttttccacttacactttccccgtcttttctgtaggaaatcgtgtcaggcgatagtgagcggccgcactcgaggtttaaacggccggccgcggtcatagctgtttcctgaacagatcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccacgctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatggagagcgacgagagcggcctgcccgccatggagatcgagtgccgcatcaccggcaccctgaacggcgtggagttcgagctggtgggcggcggagagggcacccccgagcagggccgcatgaccaacaagatgaagagcaccaaaggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtgatgggcaccggcttccccgaggacagcgtgatcttcaccgacaagatcatccgcagcaacgccaccgtggagcacctgcaccccatgggcgataacgatctggatggcagcttcacccgcaccttcagcctgcgcgacggcggctactacagctccgtggtggacagccacatgcacttcaagagcgccatccaccccagcatcctacagaacgggggccccatgttcgccttccgccgcgtggaggaggatcacagcaacaccgagctgggcatcgtggagtaccagcacgccttcaagaccccggatgcagatgccggtgaagaataactgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 17 - DNA, 7733 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagacgcggtacctagctaggtagcaattgaccggtcaagatggcggccaacaacaacaacaacaacaacaacaacaacaacaacaacaacaacaagaagatggcggcaacaacaacaacaacaacaacaacaacaacaacaacaacaaccaacaacaagatggcggccaacaacaacaacaacaacaacaacaagaagatggcggcaacaacaacaacaacaacaacaacaaccaagatggcggccaacaacaacaagaagatggcggcaacaacaacaaccaagatggcggccaacaacaacaagaagatggcggcaacaacaacaaccaagatggcggcacgcgtcggtccggctagccgtacgctccttagcgacgaaatctactgcccccctgagagccaccatggcttggggtcctacgctgtgcaggccaagtttggagattacaacaaagaaggccgccatggtgggcacctcagctctgagcggctcatccgccaccatgggttggaccagcacaaacttaccagggaccgccgccatggccggacccaggcgtgccaccatggacaccgtgggttgcgccgccatggtgctctgttggagtgccaccatggtgctcaggacctgggccgccatggaatacctgataactgataagccaccatgggaacagacctttggcttggagttgacgcccttggactcaacatttacgaggccgccatggagttcaccccaaagattggctttccttggagtgaaatcaggaacatctctgccaccatggaaaagtttgtcatcaagcccatcgacaaggccgccatggactttgtgttttacgccccacgtctcacagccaccatggggaccctgcagctcgccgccatggaccacgagttgtacgccaccatggggaagcctgacaccgccgccatggagcagacgaaggccgccaccatggaggctgataagctgataagccgccatgggctggaaacagagaagaaaaggagagaaaccgtggagagagagaaagagcgccaccatggcgagaaggaggagttgttgctgcggctgcaggactacgaggagaagacaagccgccatgggagagacctctcggagcagattcagaggggccaccatggggaggaggagaggaagcgggcacaggagggccgccatggcccagaggctgaccgccaccatggactgcgggctaagggccgccatgggagacaggcggtgggccaccatgggagccaggagcagcgccgccatgggctacctgataactgataagccaccatggtggaagaggcgcggaggcgcaaggaggacgaagttgaagagtggcagcaagccgccatggaagcccaggacgacctggtcaagaccaaggaggagctgcacctggtgccggccaccatggcgccaccaccaccacccgtgtacgagccggccgccatggacgtccaggagagcttgcaagacgagggtgccaccatggcgggctacagcgcagccgccatggctgacggcatccgggccaccatggacgaggagaagcgtgccgccatggcagagaagaacgaggccaccatggggcctgataagctgataagccgccatggggcccgagacgagaacaagaggacccacaacgacatcatccacaacgagagccaccatggaggccgggacaagtacaagacgctgcggcagatccggcagggcaacaccagccgccatggcgacgagttcgaggccctgcaacagccaggccaccatggagggcagaggggtgctcatagcgggcgctgccgccatggccacgcttgtgtctgccaccatggaagtctcggaactcgccgccatggcagttcctttcgaagccaccatggcaacagaaacattcgccgccatggaccacctgataactgataagccaccatggttgcaatcgtgccaagcaggcctgattctcgcgattactcgcgaatcaccgccgccatggtgctgggagcaggactcattgaattacggaaaacgcctgtcaagtctcaggccaccatggggaactggcctgtgtcatacaagagtcaggccgccatggggaaacgtggcaggacttccatctgtgccgccaccatggtgtattcgaaacgagccgccatggattttctcatctctgccaccatggcatctttgtacattgccgccatgggaggggtcaaaattgccaccatggtggctgataagttgatagtaaccgccatggtgtttcatccagtcgccaccatgggctggcagagagcagccgccatggcagcgtcagtggtggccaccatggcttggatttttttttttgttttttttttttttgctcaacaattttacaacacattgtgtcgagcccgggaattcgtttaaacctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 18 - DNA, 6679 bp, Artificial Sequence
  Synthetic
aacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagctgatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagaattttgtaatacgactcactatagggcggccgggaattcgtcgactggatcttgtacattcgaacgccgccatgggcgctgatgatgttgttgattcttctaaatcttttgtcatggaaaacttttcttcgtaccacgggactaaacctggttatgtggattccattcaaaaaggtatacaaaagccaaaatctggtacacaaggaaattatgacgatgattggaaagggttttatagtaccgacaataaatacgacgctgcgggatactctgtggataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatagtgaactagttccggatctagagcggccgcactcgaggtttaaacggccggccgcggtcatagctgtttcctgaacagatcccgggtggcatccctgtgacccctccccagtgcctctcctggccctggaagttgccactccagtgtccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattgggtacgcggccagcttggctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcccgggagcttgtatatccattttcggatctgatcaagagacacgtacgaccatgaaaaagcctgaactcaccgcgacgtctgttgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaatttagcgagagcctgacctattgcatctcccgccgtgcacagggtgtcacgttgcaagacctgcctgaaaccgaactgcccgctgttctgcaaccggtcgcggaggccatggatgcaatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagctgcagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaatttt

SEQ ID NO: 19 - DNA, 870 bp, Artificial Sequence
  Synthetic
cgcggatccaccggtcaattgtatcaactctgagatgcaggtacatccagctgatgagtcccaaataggacgaaacgcgcttcggtgcgtcctggattccactgctatccactattcatctacttgcactgcacccgataccctgtcaccggatgtgctttccggtctgatgagtccgtgaggacgaaacaggactggaacgtactacgacaggaacttgtcctgagatgcaggtacatcccactgatgagtcccaaataggacgaaacgcgcttcggtgcgtctgggattccactgctatccacacgcgtcggtccgaagcttgtcgaccgccggtgcaaagatctgaattcacctgatagctgatagctgatagcccggggtctctgtggatagaccagagcggagcctgggagctctctggctatctacggaacccactgcttaagcctcaataccgcttgccttgagtgcttcaagtagtgtgtgccgaacacaagctcacgacccactacacaagctcacgacccactacacaagctcacgacccactacacgagcttggggcgcgtggtggcggctgcagccgccaccacgcgccccggatcggagattgtcaggagctaaggaagctaaacaacgactacagcaggcttttgcaaaaagctccaccacggcccaacgttgggccgtggtggagcttggattgtacttgcactgcatacacaacgagatcggaacgtactacgacaggaactacgaccctgcggtccaccacggccgatatcacggccgtggtggaccgcagggaagaacaacgtctccgatctttggtaccaacacatctagacaaagtactggcgcgcccaa

SEQ ID NO: 20 - DNA, 4950 bp, Artificial Sequence
  Synthetic polynucleotide
gcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggattcgaaagatctgccaccatacgttgccgcgcagcggactgcccgccaggatatggatcctgatgatgttgttgattcttctaaatcttttgttatggaaaacttttcttcgtaccacgggactaaacctggttatgtggattccattcaaaaaggtatacaaaagccaaaatctggtacccaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgccgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatctctttgtgaaggaaccttacttctgtggtgtgacataattggacaaactacctacagagatttaaagctctaatgactcgagccatgggacccacacttttctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagctttagtcctgttcctcagctacaaaatggacacaatttccagcagggtctctgagggcaaattcccttccccaaggttgttcaccaatttctgtcatggctgggccagaggcatccctgaaatttgtgctgactacttctgaccattctgcataaagctcatctaggcctctgacccagacccaagcaagggtgttgtcagggacaacttggtcctgaactgctgagatgaagagggtgacatcatctctgacaacaccagcaaaatcatcttcaacaaagtctctggagaatcctaatctgtcagtccagaactctacagcccctgcaacatcccttgctgtgaggactgggactgcagaagtgagtttggccatgatggctcctcctgtcaggagaggaaagagaagaaggttagtacaattgctatagtgagttgtattatactatgcttatgattaattgtcaaactagtgggttcatagtgccacttttcctgcactgccccatctcctgcccaccctttcccaggcatagacagtcagtgacttacccttgtacagctcatccattcccagagtaattcctgctgctgtcacaaactccaggaggaccatgtggtctcttttctcattagggtctttggacagagcagattgagtgctgagatagtgattatctgggaggagaactgggccatcaccaataggggtgttctgctggtaatggtctgccagttggacagatccatcctcaatgttgtgtctaatcttgaaattagccttaattccattcctctgcttatctgccataatgtaaacattgtgagaattatagttgtactccagcttgtgacccagaatgtttccatcttccttaaaatcaatgcctttcagctcaattctgttaaccagtgtatcaccttcaaacttcacttctgcccttgtcttataatttccatcatccttaaagaagattgtcctctcctgaacataaccttctggcattgcagatttaaagaagtcatgctgcttcatgtggtcagggtatctgctgaaacattgaacaccataagtcagggtggtcaccagagttggccaaggcactggcagctttcctgttgtacaaatgaacttcagagtcagctttccataagttgcatctccttcaccttcaccagacacagagaatttgtggccattcacatcaccatccagctcaaccagaattgggacaacaccagtaaagagttcttctcccttgctcatggtggcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagctgatctgacggttcactaaacgagctctgcttatatagacctcccaccgtacacgcctaccgcccatttgcgtcaatggggcggagttgttacgacattttggaaagtcccgttgatttactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatca

SEQ ID NO: 21 - DNA, 7241 bp, Artificial Sequence
  Synthetic
agatctgcgcagcaccatggcctgaaataacctctgaaagaggaacttggttaggtacctaccggaaggaacccgcgctatgacggcaataaaaagacagaataaaacgcacggtgttcttataatggttacaaataaagcaatagcatcacaaatttcacaaataaagcatttttttcactgcattctagttgtggtaataaaatatctttattttcattacatctgtgtgttggttttttgtgtgtggcctcccaaagtgctgggattacaggcatgagccatcgagcccaacccaattttttttttttttaattttactttctgcaatcattcatccattcagccagtgcggtatttctgaggtgtgttcgatcgcggatccatgcctgccgcagtacagttgtgagccaaatgagactgagactagttcccgccctccaagagcttgcaagacccgcagtggcgtaaaaacactaacatcttttagtgatcgattctgcactccaggggttttcaatctactacaagagtgaataagagttcgcctttgtctgatatctgttgtcattctctctcgcttctttaactgattttttctcagctaataaaacatccacccacaaccccccgaacgcccgcaaacaccaggccactctagcaaaacctctctcactccgcctgcgcaatccagctgacttccggttacagataaccacgtgattgggaacccttgctgcgcatgtctagtaggaagtcggactataccactttccctacggaaggggtacttttttatgtttttaagtttaaaaccgatttctgatatttgacttttatcatttcaggcctatatggaggctatgagtgagtttagtgtggcagaagatgaaagaaccggacaggaatacggacgaaattggagcagggtttgggctctccccttcgcagataatcggaggagccgggcccgagcgagctctttcctttcgctgctgcggccgcagccgtgaggtgagggcgagctggtctccatcaggcgctgacgcgtgtcgacaagggactgtcggtcttgggaccgcagctggggttgggggagatgaaatggaggccgccctaaagcggccggtcccggggtttggggtaggccggagcactttcgtcccgggcctccggagtgagggggggcggggagcgtcgcagcaactgagaccaggaaaagtctgccccggctggtgccgcaccgcacacgtgtccggtcgacccacgcgagcagagcaaacggagcgaacaagaccaagccgtgggccctttcttgcttggcacacccggagcggagccgatctctgctttcacgtgatgtagggcaagcctagtgtaggccccaggcctccgactgccgagagaggtgatctctaactcttgactccattcactcctttggcctctcataaaggaaatctctgcgaatagccgaacgaggcttgttactgtgataaaacagggaaataagcccagaaaacagagtaacttgcctgcattcctagactagaaatcaggtctactcacctcgaatattctttaaacgctgagtaccagaaatggcataacccccctattcaatccaataagtccttggcttgactttccagaggagaaatgcgaacatgaggctccgagaggtgaaggcatagcgtgggttttgaagtcttaaacccaagggggccagctgcatagcccagagccttaaagatgatttagggaagagtcttatttcgcggctgtggtgtgggtcacaaagggcaggtcttgatggggacgttcattcttgcccaggattggctttcagagtctaatcatgttttctgtgtgtctagtatcctcaggcttcagaagaggctcgcctctagtgtcctccgctgtggcaagaagaaggtctggaccggtcccacgcgtcaattggaaaacttacgctgagtacttcgatctccctacggcaagctgaccctgaagttcaacagatctcgccgccatgggagctgatgatgtggttgattcttcgaaatcttttgtcatggaaaacttttcttcgtaccacgggacgaaacctggttatgtggattccattcaaaaaggcatacaaaagccaaaatctggtacacaaggaaactatgacgatgattggaaagggttttatagtaccgacaacaaatatgacgctgcgggatactctgtggataatgaaaacccgctctctggaaaagctggaggcgtggtcaaagtgacgtatccaggactgacgaaggttctcgcactaaaggtggataatgccgaaactattaagaaagagttaggtttaagtctcactgaaccgctcatggagcaagtcggaacggaagagtttatcaaaagattcggtgatggtgcttcgcgtgtagtgctcagccttcccttcgctgaggggagttctagcgttgagtacatcaacaactgggaacaggcgaaagcgttaagcgtagaacttgagattaactttgaaacccgtggaaaacgtggccaagatgcgatgtatgagtatatggctcaagcctgtgcaggaaatcgtgtcaggcgatagtgaactagtatccggaatctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 22 - DNA, 6326 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcccgaccggtcaacaacgtctcagatcttcccccaaggcgcgccgaactctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctaggcgatcgctcgagcccgggaattcgtttaaacctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 23 - DNA, 9195 bp, Artificial Sequence
  Synthetic
agatctgcgcagcaccatggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttgattcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcccgaccggtcaacaacgtctcagatcccggtccgactagtcgtacgcaccggcggtcgaccacgtgcctgcaggaaagtgcgcacggtcaccgagctcaaaacgccaagaacctcatcatcttccctacggcaagctgaccctgaagttcatcaactacacaaatcagcgatttccagctcagcgggacccgcatccggaggcgcgccgaactctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctaggcgatcgtctagagcctgacctagctgacctagccgccaccatgcagaggctgcaggtagtgctgggccacctgaggggtccggccgattccggctggatgccgcaggcagcgccttgcctgagatctactagtggtgacctagctgacctagccgtcaccatggaccctgttgtgctgcaaaggagagactgggagaaccctggagtcacccagctcactgaacttaaccctagccctgccaccatggcttggaggaactccgaggaagccaggactgacagtaacagtaggcagcgccgccatggcaacggagagtggaggtttgcctggtttgacgctaaggatagtgtggccaccatggggctggagtgcgacctcccagaggcggctgacgttaaagttagcagcagccgccatgggcacggctacgacgcgcccatctacagtgacgttagcactaagatcgccaccatggccccttttgtgcccaccgagaacccgactaactgtagcagtgacacctgccgccatggcgagagctggctgcaagaaggacagactaagattagctgtgacggagccaccatggccttccacctctggtgcaacggcaggtgtaacggtagcggtgaagacagccgccatggctccgagttcgacctctctgccttccgtaacgctgaagataacagggccaccatggtggtgctcaggtggtccgacggcagctatagggctgaccgtaacatgtgccgccatggtggcatcttcagggacgtcagcctgcttagcactgagactaaccaggccaccatggtccacgttgccacgaggttcaacgacgatagcagtgaagctaagctgggccgccatgggcagatgtgtggagaactcagagagtctgacagtagcactaagagcgccaccatgggcgagacccaggtggcctctggcacagctgactttagagctaagatcagccgccatggaggaggctacgccgacagagccacccttgagcttagcgttaagaacgccaccatggggtctgccgagacccccaacctctacagtaacgttagggctgagcacagccgccatggcacgctcatcgaagccgaagcctgcgataacggtgacagtagagtcgccaccatggacggcctgctgctgctcaacggcaagcctaagcttgacagtagagtcagccgccatgggcaccatcctctgcacggacaagtcattaaggctgagactagggtggccaccatggtgctcacgaagcagaacaacttcaacgctaagagtgactctagctaccgccgccatggtctctggtacaccctgtgcgacaggaataaccttagggttgacgaggccaccatggtcgagacacacggcatggtgcccacgaataagcttagagctgaccccagccgccatggtgccatgtccgagagagtcaccaggattaagcatagagctgagaacgccaccatggtcatcatctggtctctgggcaacgagtctaagcatagagctgaccacggccgccatggcaggtggatcaagtctgccgaccccagtagacctgagcataacgaagccaccatggcagacaccacagccacagacatcatctgtagcattgaggctaaggtcggccgccatgggcccttccctgctgtgcccaagtggagtagcactgagtgtaactctgccaccatggaaacgagacctctcatcctgtgcgagtatagacctgaaagtaaacccggccgccatggctttgccaagtactggcaagccttcactgagtatagcactaagcaagccaccatggcccgcgactgggtggaccagtcactcattgagtatagcgataacggcagccgccatggtgcctacggaggagactttggcgacactgagaatagcactaagttcgccaccatgggcctggtctttgccgaccggactccgcctgaccctagcactaaggccagccgccatggacagttcttcccgttcacgctgtctggtgaaactaacgatagcacagccaccatggtcttcagacactccgacaacgagctgcttgactgtaaggctaggctgggccgccatggtctggcttctggcgaggtgcctctggctgaggctaatcatagaaaggccaccatggaactgcccgagctgcctcagccagagtctgacgataaccttaggctcagccgccatggggttcagcccaacgcaacagcttggtctgagcctagccataactctgccaccatggagtggaggctggccgagaacctctcggttgaccttagggctaactctcgccgccatggtcacctcacaacatccgaaatggagtttgacattaggcttaacaacgccaccatggagttcaacaggcagtctggcttcctgtctgagattaggactaaagacagccgccatggcctctctcctctccgagacctgttcactagggctgagcttaacactgccaccatggtgtcagaggccaccaggatcgacccaaatagttgtgaggataagtggagccgccatggacactaccaggccgaggctgccctgcttagctgtgaagctaaccagcgaatgcctggggctctcatcaccacagcccacgcttgtagccctgaagctaagacagcgaatgcctggggcaagacctacagaatcgacggccataggaaacctagagcggccgctggccgcaataaaatatctttattttcattacatctgtgtgttggttttttgtgtgaggatctaaatgagtcttcggacctcgcgggggccgcttaagcggtggttagggtttgtctgacgcggggggagggggaaggaacgaaacactctcattcggaggcggctcggggtttggtcttggtggccacgggcacgcagaagagcgccgcgatcctcttaagcacccccccgccctccgtggaggcgggggtttggtcggcgggtggtaactggcgggccgctgactcgggcgggtcgcgcgccccagagtgtgaccttttcggtctgctcgcagacccccgggcggcgccgccgcggcggcgacgggctcgctgggtcctaggctccatggggaccgtatacgtggacaggctctggagcatccgcacgactgcggtgatattaccggagaccttctgcgggacgagccgggtcacgcggctgacgcggagcgtccgttgggcgacaaacaccaggacggggcacaggtacactatcttgtcacccggaggcgcgagggactgcaggagcttcagggagtggcgcagctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagcgacccgcttaaaagcttggcattccggtactgttggtaaagccaccatggccgatgctaagaacattaagaagggccctgctcccttctaccctctggaggatggcaccgctggcgagcagctgcacaaggccatgaagaggtatgccctggtgcctggcaccattgccttcaccgatgcccacattgaggtggacatcacctatgccgagtacttcgagatgtctgtgcgcctggccgaggccatgaagaggtacggcctgaacaccaaccaccgcatcgtggtgtgctctgagaactctctgcagttcttcatgccagtgctgggcgccctgttcatcggagtggccgtggcccctgctaacgacatttacaacgagcgcgagctgctgaacagcatgggcatttctcagcctaccgtggtgttcgtgtctaagaagggcctgcagaagatcctgaacgtgcagaagaagctgcctatcatccagaagatcatcatcatggactctaagaccgactaccagggcttccagagcatgtacacattcgtgacatctcatctgcctcctggcttcaacgagtacgacttcgtgccagagtctttcgacagggacaaaaccattgccctgatcatgaacagctctgggtctaccggcctgcctaagggcgtggccctgcctcatcgcaccgcctgtgtgcgcttctctcacgcccgcgaccctattttcggcaaccagatcatccccgacaccgctattctgagcgtggtgccattccaccacggcttcggcatgttcaccaccctgggctacctgatttgcggctttcgggtggtgctgatgtaccgcttcgaggaggagctgttcctgcgcagcctgcaagactacaaaattcagtctgccctgctggtgccaaccctgttcagcttcttcgctaagagcaccctgatcgacaagtacgacctgtctaacctgcacgagattgcctctggcggcgccccactgtctaaggaggtgggcgaagccgtggccaagcgctttcatctgccaggcatccgccagggctacggcctgaccgagacaaccagcgccattctgattaccccagagggcgacgacaagcctggcgccgtgggcaaggtggtgccattcttcgaggccaaggtggtggacctggacaccggcaagaccctgggagtgaaccagcgcggcgagctgtgtgtgcgcggccctatgattatgtccggctacgtgaataaccctgaggccacaaacgccctgatcgacaaggacggctggctgcactctggcgacattgcctactgggacgaggacgagcacttcttcatcgtggaccgcctgaagtctctgatcaagtacaagggctaccaggtggccccagccgagctggagtctatcctgctgcagcaccctaacattttcgacgccggagtggccggcctgcccgacgacgatgccggcgagctgcctgccgccgtcgtcgtgctggaacacggcaagaccatgaccgagaaggagatcgtggactatgtggccagccaggtgacaaccgccaagaagctgcgcggcggagtggtgttcgtggacgaggtgcccaagggcctgaccggcaagctggacgcccgcaagatccgcgagatcctgatcaaggctaagaaaggcggcaagatcgccgtgtaataattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgac

SEQ ID NO: 24 - RNA, 13 nt, Artificial Sequence
  Synthetic
gccgccrccaugg

SEQ ID NO: 25 - RNA, 7 nt, Homo sapiens
accaugg

SEQ ID NO: 26 - protein, 9 aa, Artificial Sequence
  Synthetic
XXXXXXXXX

SEQ ID NO: 27 - protein, 9 aa, Homo sapiens
RLRVLSGHL

SEQ ID NO: 28 - RNA, 31 nt, Artificial Sequence
  Synthetic
uggagcagaggcucuggcagcuuuugcagcg

SEQ ID NO: 29 - RNA, 21 nt, Artificial Sequence
  Synthetic
gccaaggagccagagagcaug

SEQ ID NO: 30 - RNA, 11 nt, Artificial Sequence
  Synthetic
gccaaggagcc

SEQ ID NO: 31 - RNA, 5 nt, Artificial Sequence
  Synthetic
auuua

SEQ ID NO: 32 - RNA, 9 nt, Artificial Sequence
  Synthetic
uuauuuann

SEQ ID NO: 33 - RNA, 4 nt, Artificial Sequence
  Synthetic
auuu

SEQ ID NO: 34 - RNA, 16 nt, Artificial Sequence
  Synthetic
ggcucuuuucagagcc

SEQ ID NO: 35 - RNA, 6 nt, Artificial Sequence
  Synthetic
uuuuau

SEQ ID NO: 36 - RNA, 7 nt, Artificial Sequence
  Synthetic
uuuuuau

SEQ ID NO: 37 - RNA, 7 nt, Artificial Sequence
  Synthetic
uuuuaau

SEQ ID NO: 38 - RNA, 9 nt, Artificial Sequence
  Synthetic
uuuuuuauu

SEQ ID NO: 39 - RNA, 7 nt, Artificial Sequence
  Synthetic
uuuuauu

SEQ ID NO: 40 - RNA, 11 nt, Artificial Sequence
  Synthetic
uuuuuauaaag

SEQ ID NO: 41 - RNA, 70 nt, Human herpesvirus 4
gggggucuuaguggaagugacgugcugugaauacagguccauagcaccgcuauccacuaugucucgcccg

SEQ ID NO: 42 - RNA, 24 nt, Human herpesvirus 4
ucuuaguggaagugacgugcugug

SEQ ID NO: 43 - RNA, 24 nt, Artificial Sequence
  Synthetic
cacagcacgucacuuccacuaaga

SEQ ID NO: 44 - RNA, 700 nt, Artificial Sequence
  Synthetic
ccugucaccggauguguuuuccggucugaugaguccgugaggacgaaacaggggaaccauggcacagcacgucacuuccacuaagacaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagcccaaaggcucuuuucagagccccccua

SEQ ID NO: 45 - RNA, 624 nt, Artificial Sequence
  Synthetic
uccacuaagacaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagcccaaaggcucuuuucagagccccccua

SEQ ID NO: 46 - RNA, 53 nt, Human immunodeficiency virus type 1
uuacacaccagggcagggaucagauauccacugaccuuuggauggugcuucaa

SEQ ID NO: 47 - RNA, 24 nt, Human immunodeficiency virus type 1
acugaccuuuggauggugcuucaa

SEQ ID NO: 48 - RNA, 24 nt, Artificial Sequence
  Synthetic
uugaagcaccauccaaaggucagu

SEQ ID NO: 49 - RNA, 22 nt, Artificial Sequence
  Synthetic
ggggaagggggugggggugggg

SEQ ID NO: 50 - RNA, 22 nt, Artificial Sequence
  Synthetic
ccccacccccacccccuucccc

SEQ ID NO: 51 - RNA, 761 nt, Artificial Sequence
  Synthetic
ccgcggcugagaugcagguacaucccacugaugagucccaaauaggacgaaacgcgcuucggugcgucugggauuccacugcuauccacgcggccgccaugaccaugguugaagcaccauccaaaggucaguggggaaggggguggggguggggaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagccccacccccacccccuucccc

SEQ ID NO: 52 - RNA, 639 nt, Artificial Sequence
  Synthetic
aaaggucaguggggaaggggguggggguggggaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagccccacccccacccccuucccc

SEQ ID NO: 53 - RNA, 86 nt, Homo sapiens
cguugucuauauauacccuguagaacgaauuugugugguauccguauagucacagauucgauucuaggggaauauauggucgaaug

SEQ ID NO: 54 - RNA, 23 nt, Homo sapiens
uacccuguagaaccgaauuugug

SEQ ID NO: 55 - RNA, 23 nt, Artificial Sequence
  Synthetic
cacaaauucgguucuacagggua

SEQ ID NO: 56 - RNA, 2684 nt, Artificial Sequence
  Synthetic
augaccaugggaaccaugcgccuccgcguucucucaggccaccucacacaaauucgguucuacaggguaccaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagacacaaauucgguucuacaggguauaguagguuagacaccugcuucuccccaauagaggggggggacccaaacgacagggggcgccccagaggcuaaggucggccacgccacucgcgggugggcucguguuacagcacaccagcccguucuuuucccccccucccacccuuagucagacucuguuacuuacccguccgaccaccaacugcccccuuaucuaagggccggcuggaagaccgccagggggucggccggugucgcuguaaccccccacgccaaugacccacguacuccaagaaggcaugugucccaccccgccuguguuuuugugccuggcucucuaugcuugggucuuacugccuggggggggggagugcgggggaggggggguguggaaggaaaugcacggcgcguguguacccccccuaaaguuguuccuaaagcgaggauacggaggaguggcgggugccgggggaccggggugaucucuggcacgcgggggugggaagggucgggggagggggggauggaguaccggcccaccuggccgcgcgggugcgcgugccuuugcacaccaaccccacgucccccggcggucucuaagaagcaccgcccccccuccuucauaccaccgagcaugccuggguguggguugguaaccaacacgcccauccccucgucuccugugauucucuggcugcaccgcauucuuguuuucuaacuauguuccuguuucugucucccccccccccaccccuccgccccaccccccaacacccacgucugugguguggccgacccccuuuugggcgccccgucccgccccgccaccccucccauccuuuguugcccuauaguguaguuaaccccccccgcccuuuguggcggccagaggccaggucaguccgggcgggcaggcgcucgcggaaacuuaacacccacacccaacccacugugguucuggcuccaugccaguggcaggaugcuuucggggaucgguggucaggcagcccgggccgcggcucugugguuaacaccagagccugcccaacauggcacccccacucccacgcacccccacucccacgcacccccacucccacgcacccccacucccacgcacccccacucccacgcacccccacucccacgcacccccacucccacgcacccccacucccacgcacccccacucccacgcauccccgcgauacauccaacacagacagggaaaagauacaaaaguaaaccuuuauuucccaacagacagcaaaaauccccugaguuuuuuuuuauuagggccaacacaaaagacccgcugguguguggugcccgugucuuucacuuuuccccuccccgacacggauuggcugguguagugggcgcggccagagaccacccagcgcccgaccccccccuccccacaaacacggggggcgucccuuauuguuuucccucgucccgggucgacgcccccugcuccccggaccacgggugccgagaccgcaggcugcggaaguccagggcgcccacuagggugcccuggucgaacagcauguuccccacgggggucauccagaggcuguuccacuccgacgcgggggccgucggguacucggggggcaucacgugguuacccgcggucucggggagcagggugcggcggcuccagccggggaccgcggcccgcagccgggucgccauguuucccgucugguccaccaggaccacguacgccccgauguuccccgucuccauguccaggaugggcaggcagucccccgugauagucuuguucacguaaggcgacagggcgaccacgcuagagacccccgagaugggcagguagcgcgugaggccgcccgcggggacggccccggaagucuccgcguggcgcgucuuccgggcacacuuccucggcccccgcggcccagaagcagcgcgggggccgagggagguuuccucuugucucccucccagauuuauuuaauuauuuauuauuauuuaaauuauuuauauuauuuaau

SEQ ID NO: 57 - RNA, 612 nt, Artificial Sequence
  Synthetic
cuacaggguaccaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauagucacagcacgucacu

SEQ ID NO: 58 - RNA, 61 nt, Herpes simplex virus-1
ccguggcggcccggcccggggccccggcggacccaaggggccccggcccggggccccacaa

SEQ ID NO: 59 - RNA, 20 nt, Herpes simplex virus-1
uggcggcccggcccggggcc

SEQ ID NO: 60 - RNA, 20 nt, Artificial Sequence
  Synthetic
ggccccgggccgggccgcca

SEQ ID NO: 61 - RNA, 808 nt, Artificial Sequence
  Synthetic
accauggaaccauggggccccgggccgggccgccacgaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauaguuuuuuauunnnnnnnnnnnnnnnnnnnnnnnnnnnnnnnnnnnnnnnnnnnnnnnnnnnnnnnnnnnnnnnnnnnnnnnnnnnnnnnnnnuuuuauuggggccccgggccgggccgccacggaaccuggagcagaggcucuggcagcuuuugcagcgggaaaccgccaaggagccagagagcaug

SEQ ID NO: 62 - RNA, 707 nt, Artificial Sequence
  Synthetic
cgggccgccacgaugggcgcugaugauguuguugauucuucuaaaucuuuugucauggaaaacuuuucuucguaccacgggacuaaaccugguuauguggauuccauucaaaaagguauacaaaagccaaaaucugguacacaaggaaauuaugacgaugauuggaaaggguuuuauaguaccgacaauaaauacgacgcugcgggauacucuguggauaaugaaaacccgcucucuggaaaagcuggaggcguggucaaagugacguauccaggacugacgaagguucucgcacuaaaaguggauaaugccgaaacuauuaagaaagaguuagguuuaagucucacugaaccguugauggagcaagucggaacggaagaguuuaucaaaagguucggugauggugcuucgcguguagugcucagccuucccuucgcugaggggaguucuagcguugaauauauuaauaacugggaacaggcgaaagcguuaagcguagaacuugagauuaauuuugaaacccguggaaaacguggccaagaugcgauguaugaguauauggcucaagccugugcaggaaaucgugucaggcgauaguuuuuuauunnnnnnnnnnnnnnnnnnnnnnnnnnnnnnnnnnnnnnnnnnnnnnnnnnnnnnnnnnnnnnnnnnnnnnnnnnnnnnnnnnuuuuauuggggccccgggc

SEQ ID NO: 63 - RNA, 52 nt, Artificial Sequence
  Synthetic
ccugucaccggauguguuuuccggucugaugaguccgugaggacgaaacagg

SEQ ID NO: 64 - RNA, 97 nt, Artificial Sequence
  Synthetic
ccgcggcugagaugcagguacaucccacugaugagucccaaauaggacgaaacgcgcuucggugcgucugggauuccacugcuauccacgcggccgc

SEQ ID NO: 65 - DNA, 21 bp, Artificial Sequence
  Synthetic RNA/DNA
aaacaugcagaaaaugcugtt

SEQ ID NO: 66 - DNA, 21 bp, Artificial Sequence
  Synthetic RNA/DNA
cagcauuuucugcauguuutt

SEQ ID NO: 67 - DNA, 23 bp, Artificial Sequence
  Synthetic RNA/DNA
aagcuaccuguuccauggccatt

SEQ ID NO: 68 - DNA, 23 bp, Artificial Sequence
  Synthetic
tggccatggaacaggtagctttt

SEQ ID NO: 69 - RNA, 21 nt, Artificial Sequence
  Synthetic
gaguucccgacgcguccuagc

SEQ ID NO: 70 - RNA, 21 nt, Artificial Sequence
  Synthetic
uaggacgcgucgggaacucgc

SEQ ID NO: 71 - DNA, 21 bp, Artificial Sequence
  Synthetic
aactacacaaatcagcgattt

SEQ ID NO: 72 - RNA, 21 nt, Artificial Sequence
  Synthetic
cuacacaaaucagcgauuuuu

SEQ ID NO: 73 - RNA, 21 nt, Artificial Sequence
  Synthetic
aaaucgcugauuuguguaguu

SEQ ID NO: 74 - DNA, 26 bp, Artificial Sequence
  Synthetic
aaaacttacgctgagtacttcgatct

SEQ ID NO: 75 - DNA, 21 bp, Artificial Sequence
  Synthetic RNA/DNA
cuuacgcugaguacuucgatt

SEQ ID NO: 76 - DNA, 21 bp, Artificial Sequence
  Synthetic
ucgaaguacucagcguaagtt

SEQ ID NO: 77 - DNA, 28 bp, Artificial Sequence
  Synthetic
ctacggcaagctgaccctgaagttcatc

SEQ ID NO: 78 - RNA, 22 nt, Artificial Sequence
  Synthetic
gcaagcugacccugaaguucau

SEQ ID NO: 79 - RNA, 22 nt, Artificial Sequence
  Synthetic
gaacuucagggucagcuugccg

SEQ ID NO: 80 - DNA, 24 bp, Artificial Sequence
  Synthetic
aatcgcttaccgattcagaatcgc

SEQ ID NO: 81 - DNA, 21 bp, Artificial Sequence
  Synthetic RNA/DNA
ucgcuuaccgauucagaautt

SEQ ID NO: 82 - DNA, 21 bp, Artificial Sequence
  Synthetic RNA/DNA
auucugaaucgguaagcgatt

SEQ ID NO: 83 - RNA, 7 nt, Artificial Sequence
  Synthetic
rnnaugg

(19) United States
(12) Patent Application Publication
(10) Pub. No.: US 2013/0245096 A1
(43) Pub. Date: Sep. 19, 2013

(54) COMPOSITIONS AND METHODS FOR ACTIVATING EXPRESSION BY A SPECIFIC ENDOGENOUS miRNA

(71) Applicant: Guy Abitbol, Rishon Lezion (IL)

(72) Inventor: Guy Abitbol, Rishon Lezion (IL)

(73) Assignee: NANODOC Ltd., Rehovot (IL)

(21) Appl. No.: 13/881,350

(22) PCT Filed: Oct. 27, 2011
(86) PCT No.: PCT/IL11/00837

§ 371 Date: Apr. 24, 2013

(30) Foreign Application Priority Data
Oct. 28, 2010 (IL) PCT/IL2010/000894

(51) Int. Cl. C07H 21/02; A61K 31/7088

(52) U.S. Cl. CPC C07H 21/02; A61K 31/7088
USPC 514/44 A; 536/24.5; 536/23.2; 536/23.7; 536/23.6; 435/375; 435/320.1

(57) ABSTRACT

There are provided compositions and methods for activating expression of an exogenous polynucleotide of interest only in the presence of a specific endogenous miRNA in a cell. Further provided are uses for the compositions in treatment and diagnosis of various conditions and disorders, for example by selectively activating expression of a toxin only in target cell populations.

### FIELD OF THE INVENTION

The present invention relates to compositions for activating expression of an exogenous polynucleotide of interest only in the presence of a specific endogenous miRNA in a cell. The invention further relates to uses of the compositions in treatment and diagnosis of various conditions and disorders, as exemplified by selectively activating expression of a toxin only in target cell populations.

### BACKGROUND OF THE INVENTION

Viruses are the most abundant type of biological entity on the planet and viruses appear to be the second most important risk factor for cancer development in humans. The WHO (world health organization) International Agency for Research on Cancer estimated that in 2002, ˜15% of human cancers were caused by 7 different viruses. Viruses may be oncogenic due to an oncogene in their genome. Retroviruses may also be oncogenic due to integration at a site which truncates a gene or which places a gene under control of the strong viral cis-acting regulatory element. According to the WHO in 2006 there were about 39.5 million people with HIV worldwide. Many viruses including HIV exhibit a dormant or latent phase, during which little or no protein synthesis is conducted. The viral infection is essentially invisible to the immune system during such phases. Current antiviral treatment regimens are largely ineffective at eliminating cellular reservoirs of latent viruses [1].

According to the American Cancer Society, 7.6 million people died from cancer in the world during 2007. Each tumor comprises on average 90 mutant genes [2] wherein each tumor initiated from a single founder cell [33]. The nature of and basic approaches to cancer treatment are constantly changing. Approaches to cancer treatment such as radiotherapy, surgery and inhibition of angiogenesis are not useful against many small metastases. Approaches to cancer treatment such as inhibition of cell division and destroying dividing cells have no specificity and thus cause harmful side effects that can kill the patient. Approaches to cancer treatment, such as induction of differentiation of tumor tissues, inhibition of oncogenes, virus that contains ligands against membrane receptor protein that unique to cancer cells, manipulations of the immune system and immunotoxin therapy; have a narrow therapeutic index and usually are not sufficiently effective. Approaches to cancer treatment using tumor suppressor gene and approaches to cancer treatment using toxin under promoter that is uniquely activated in cancer cells have a narrow therapeutic index, a great potential for causing harmful side effects and usually are not sufficiently effective.

Ribosome inactivating proteins (RIPs) are protein toxins that are of plant or microbial origin. RIPs inhibit protein synthesis by inactivating ribosomes. Recent studies suggest that RIPs are also capable of inducing cell death by apoptosis. Type II RIPs contain a toxic A-chain and a lectin like subunit (B-chain) linked together by a disulfide bond. The B chain is catalytically inactive, but serves to mediate entry of the A-B protein complex into the cytosol. Ricin, Abrin and Diphtheria toxin are very potent Type II RIPs. It has been reported that a single molecule of Ricin or Abrin reaching the cytosol can kill the cell [3, 4]. In addition, a single molecule of Diphtheria toxin fragment A introduced into a cell can kill the cell [5].

In mammalian cells, addition of a cap (7-methylguanosine cap) to the 5′ end of a mRNA, increases the translation of the mRNA by 35-50 fold. Further, addition of a poly(A) tail to the 3′ end of the mRNA increases the translation of the mRNA by 114-155-fold [6]. The poly(A) tail in mammal cells increases the functional mRNA half-life only by 2.6-fold and the cap increases the functional mRNA half-life only by 1.7-fold [6]. The human HIST1H2AC (H2ac) gene encodes a member of the histone H2A family. Transcripts from this gene lack poly(A) tails but instead contain a palindromic termination element (5′-GGCUCUUUUCAGAGCC-3′) that forms a conserved stem-loop structure at the 3′-UTR, which plays an important role in mRNA processing and stability [7].

RNA interference (RNAi) is a phenomenon in which dsRNA, composed of sense RNA and antisense RNA homologous to a certain region of a target gene whose function is to be inhibited, affects the cleavage of the homologous region of the target gene transcript. In mammals the dsRNA should be shorter than 31 base pairs to avoid induction of interferon response that can cause cell death by apoptosis. The Nobel Prize in Medicine and Physiology in 2006 was awarded to the RNAi field because of the huge therapeutic potential this technique harbors. However, the RNAi technology is based on a natural mechanism that utilizes microRNAs (miRNAs) to regulate posttranscriptional gene expression [8]. miRNAs are very small RNA molecules of about 21 nucleotides in length that appear to be derived from 70-90 nucleotides (nt) precursors that form a predicted RNA stem-loop structure. miRNAs are expressed in organisms as diverse as nematodes, fruit flies, humans and plants.

In mammals, miRNAs are generally transcribed by RNA polymerase II and the resulting primary transcripts (pri-miRNAs) contain local stem-loop structures that are cleaved by the Drosha-DGCR8 complex. The product of this cleavage is one or more (in case of clusters) precursor miRNA (pre-miRNA). Pre-miRNAs are usually 70-90 nucleotides long with a strong stem-loop structure, and they usually contain 2 nucleotides overhang at the 3′ end [9]. The pre-miRNA is transported to the cytoplasm by Exportin-5. In the cytoplasm, Dicer enzyme, which is an endoribonuclease of the RNase III family, recognizes the stem in the pre-miRNA as dsRNA and cleaves and releases a 21 bp dsRNA (miRNA duplex) from the 3′ and 5′ end of the pre-miRNA. The two strands of the duplex are separated from each other by the Dicer-TRBP complex and the strand that has thermodynamically weaker 5′ end is incorporated into the RNA induced silencing complex (RISC) [10]. This strand is the mature miRNA. The strand, which is not incorporated into RISC is called miRNA*strand and it is degraded [8]. The mature miRNA guides RISC to a target site within mRNAs. If the target site is near perfect complementarity to the mature miRNA, the mRNA will be cleaved at a position that is located about 10 nucleotides upstream from the 3′ end of the target site [10]. After the cleavage, the RISC-mature miRNA strand complex is recycled for another activity [11]. If the target site has lower complementarity to the mature miRNA the mRNA will not be cleaved at the target site but the translation of the mRNA will be suppressed. Although about 530 miRNAs have been identified so far in human it is estimated that vertebrate genomes encode up to 1,000 unique miRNAs, which are predicted to regulate expression of at least 30% of the genes [12], and FIG. 1.

MicroRNAs seem to play a crucial role in the initiation and progression of human cancer, and those with a role in cancer are designated as oncogenic miRNAs (oncomiRs) [12]. In lung cancer, which is one of the most common cancers of adults in economically developed countries, the expression of the miRNA cluster miR-17-92 is strongly upregulated; miR-17-92 predicted targets are PTEN and RB2, two known tumor suppressor genes [8]. In papillary thyroid carcinoma (PTC) the three miRNAs: miR-221, miR-222 and miR-146 are accumulated at a much higher level than in matching healthy tissues [8]. In glioblastoma multiforme (GBM), the most common form of brain cancer, miR-221 and miR-21 are accumulated at a much higher level than in normal tissues [8]. In B-cell-derived lymphomas, cancer of the lymphocytes, miR-155 is accumulated at a much higher level than in normal lymphoid cells [8]. In metastatic breast cancer, the transcription factor Twist, upregulates miR-10b expression compared to healthy or nonmetastatic tumourigenic cells; the target of miR-10b is HOXD10, and reducing in HOXD10 level results in higher level of RHOC, which stimulates cancer cell motility [8].

Genome-wide screens, enabled by computational approaches and high-throughput validation, have discovered about 141 microRNA precursors encoded by viruses [34, 35], a major part of these microRNAs is encoded by the herpes virus family which includes a number of human oncogenic viruses like Herpes Simplex virus, Kaposi Sarcoma Herpes Virus or Epstein Barr virus [13]. Many viral miRNAs are located within clusters in and around genomic regions associated with latent transcription [20]. Three a-herpes viruses, herpes simplex virus-1 (HSV-1) and Marek disease virus-1 and 2 (MDV-1 and MDV-2), have been shown to encode miRNAs close to and within the minor latency-associated transcript, a non-coding RNA detected during latent infections of all three viruses [20]. Multiple miRNAs have been identified within two genomic regions of the γ-herpesvirus Epstein-Barr virus and are expressed during latent infection of transformed B cell lines [20]. In murine γ-herpesvirus-68 (MHV-68), tRNA-like transcripts previously identified as latency markers were found to encode a number of miRNAs, whereas the majority of the miRNAs expressed by Kaposi sarcoma-associated herpesvirus (KSHV) are processed from a single transcript also associated with latent gene expression [20]. Other studies suggest the role of HIV-encoded microRNAs in affecting and/or maintaining a latent infection [1, 14].

Many viruses that cause cancers encode miRNAs and are capable of causing latent infection. For example, KSHV virus causes Kaposi's sarcoma cancer and encodes 13 miRNAs [13]. For example, SV40 (Simian vacuolating virus 40) has the potential to cause tumors, but most often persists as a latent infection, SV40 regulates the expression of its large T antigen via two miRNAs encoded directly antisense to the gene, expression of these miRNAs leads to cleavage of the large T antigen transcript [20]. For example, EBV encodes 23 miRNAs and expression of EBV miRNAs was observed in B cells Burkitt's lymphoma, nasopharyngeal carcinoma cells infected with EBV and EBV-associated gastric carcinomas (EBVaGCs) [13, 21]. For example, HCMV encodes 15 miRNAs and recent studies indicate the presence of genome and antigens, of HCMV in tumor cells (but not in adjacent normal tissue) of more than 90% of patients with certain malignancies, such as colon cancer, malignant glioma, prostate carcinoma, and breast cancer [36]. Moreover, detection of HCMV in different histological types of gliomas revealed that HCMV-positive cells in glioblastoma multiforme were 79% compared to 48% in lower grade tumors [36]. HCMV may increase the malignancy of the tumor cells, because they share many interest (e.g. nucleotide synthesis, DNA replication, evading from the immune system and evading from apoptosis). Current antiviral treatment regimens are largely ineffective at eliminating cellular reservoirs of latent viruses [1].

Some viral miRNAs are orthologs (genes in different species that are similar to each other since they originated from a common ancestor) of oncomiRs (miRNAs known to be involved in Cancer) [35]. Example of an orthologous viral miRNA is KSHV-miR-K12-11 of KSHV that is ortholog of hsa-miR-155, which is over expressed in: B-cell lymphomas, leukemia, pancreatic cancer and breast cancer [35]. Another example is EBV-miR-BART5 of EBV that is ortholog of hsa-miR-18a/b. hsa-miR-18a/b is encoded from hsa-miR-17-92 cluster that is over expressed in: lung cancer, anaplastic thyroid cancer cells and human B-cell lymphomas [35].

Human herpes virus 6 (HHV6) has been identified as a possible etiologic agent in multiple sclerosis, myocarditis, encephalitis and febrile seizures. Investigation of temporal lobectomy specimens showed evidence of active HHV6B replication in hippocampal astrocytes in about two-thirds of patients with MTS (mesial temporal sclerosis) [37]. HHV6 is a member of the betaherpesviridae (subfamily of the herpesviridae) which also includes HCMV (which contains 15 miRNAs) and therefore HHV6 may contain also many miRNAs.

Several therapeutic potentials of miRNAs have been proposed. One approach is to logically build microRNAs or short-hairpin RNAs (shRNAs) against ultra conserved regions in the viral transcripts or in the oncogene transcripts of a target cell [8].; however in this approach, the cleavage of the viral transcripts or the oncogene transcripts will usually not kill the target cell. Other approach is to block oncogenic or viral miRNAs by Anti-miRNA oligonucleotides (AMOs). AMOs have complementary sequences to miRNAs and contain chemical modifications to attain strong binding that can titrate away the miRNAs, one type of modifications is 2′-O-methylation of RNA nucleotides and other type of modifications is locked nucleic acid (LNA) DNA nucleotides [8]. However this approach has at least two problems: First, the blocking of the oncogenic or viral miRNAs by AMOs will usually not kill the target cell, and secondly AMOs are not capable of being transcribed in the cell and therefore AMOs need to be inserted to each target cell at huge amount for titrating away most of the miRNAs copy number. Another approach, as disclosed in, for example, WO 07/00068 is directed to a gene vector and comprising a miRNA sequence target and its use to prevent or reduce expression of transgene in a cell which comprises a corresponding miRNA. Also disclosed, for example, in WO 2010/055413, a gene vector adapted for transient expression of a transgene in a peripheral organ cell comprising a regulatory sequence operably linked to a transgene wherein the regulatory sequence prevents or reduces expression of said transgene in hematopoietic lineage cells.

There is therefore a need for developing new compositions that are potent, reliable, specific and safe to use and that are capable of selectively expressing and/or activating an exogenous protein of interest only in specific target cells that contain a specific endogenous miRNA and not in any other cell, which does not contain that specific endogenous miRNA. The compositions should preferably be capable of selectively killing the target cells that contain the specific endogenous miRNA, without any effect on other cells, which do not contain the specific endogenous miRNA.

### SUMMARY OF THE INVENTION

According to some embodiments, there are provided compositions for expressing an exogenous protein of interest in response to the presence of a specific endogenous cellular or viral miRNA in a cell. The compositions comprise or transcribe an exogenous RNA molecule that is an RNA molecule that comprises:
(a) a sequence encoding the exogenous protein of interest;
(b) an inhibitory sequence that is capable of inhibiting the expression of the exogenous protein of interest; and
(c) a binding site that is of sufficient complementarity to the mature miRNA strand of the specific endogenous miRNA to direct cleavage of the exogenous RNA molecule at a cleavage site. The predetermined target cleavage site is designed to be located between the inhibitory sequence and the sequence encoding the exogenous protein of interest.

Thus, in the presence of the specific endogenous miRNA in the cell, the exogenous RNA molecule is cleaved by the specific endogenous miRNA at the cleavage site and the inhibitory sequence is detached from the sequence encoding the exogenous protein of interest, such that the exogenous protein of interest is capable of being expressed.

In some embodiments, the exogenous protein of interest may be selected from, but is not limited to: protein toxins, Ricin, Abrin and Diphtheria toxin, fusion protein comprising protein toxins, and the like. The specific endogenous miRNA may be selected from any miRNA expressed in the cells, such as, for example, but not limited to a cellular miRNA, an oncogenic miRNAs, a viral miRNA, and the like, or any combination thereof. The inhibitory sequence can be located downstream or upstream from the cleavage site.

According to some embodiments, the inhibitory sequence that is located upstream from the cleavage site may be, for example, but is not limited to a plurality of initiation codons, wherein each of the initiation codons may be located within a Kozak consensus sequence (or any other translation initiation element) and wherein each of the initiation codons and the sequence encoding the exogenous protein of interest are not in the same reading frame. In such a setting, these initiation codons suppress the expression of the exogenous protein of interest. In another embodiment of the invention, the inhibitory sequence that is located upstream from the cleavage site may be, for example, but is not limited to: a sorting signal, an RNA localization signal for subcellular localization, a ubiquitin degradation signal, an AU-rich element (ARE), a recognition site for translation repressor, a secondary structure that is sufficient to block ribosome scanning, and the like, or combinations thereof. In one exemplary embodiment, the exogenous RNA molecule comprises a first sequence at the region of the inhibitory sequence, which is located immediately upstream from the cleavage site, wherein this first sequence is capable of binding to a second sequence that is located immediately downstream from the cleavage site. Hence, in the intact exogenous RNA molecule, the first and second sequences form a secondary structure that may block ribosome scanning, and particularly, in the cleaved exogenous RNA molecule, the second sequence may form an internal ribosome entry site (IRES) structure.

According to further embodiments, the exogenous RNA molecule sequence, having its inhibitory sequence located upstream from the cleavage site may also include a sequence or component that is capable of effecting the cleavage, directly or indirectly, of the exogenous RNA molecule at a location which is upstream from the inhibitory sequence. This may therefore reduce the efficiency of translation in the intact exogenous RNA molecule.

According to additional embodiments, the composition of the invention may further comprise one or more additional structures that may increase the efficiency of translation of the exogenous RNA molecule which may be cleaved at the 5′ end. The one or more additional structures may be, for example, but are not limited to: a nucleotide sequence that is capable of forming circularization of the cleaved exogenous RNA molecule which may therefore increase the efficiency of translation of the cleaved exogenous RNA molecule.

According to some embodiments, the compositions of the invention may be used in various applications, methods and techniques, such as, for example, but not limited to: regulation of gene expression, treatment of various conditions and disorders, including various diseases diagnostics of various conditions and disorders, such as, for example, health related conditions, formation of transgenic organisms, suicide gene therapy for treatment of proliferative disorders such as, for example, cancer; suicide gene therapy for treatment of: genetic, infectious diseases such as HIV, and the like.

According to some embodiments, there is provided a composition comprising one or more polynucleotides for directing expression of an exogenous protein of interest only in a cell expressing a specific endogenous miRNA, said one or more polynucleotides encoding an exogenous RNA molecule, which comprises: a sequence encoding for the exogenous protein of interest; an inhibitory sequence that is capable of inhibiting the expression of the exogenous protein of interest; and a binding site for said specific endogenous miRNA, whereby only in the presence of said specific endogenous miRNA, the exogenous RNA molecule is cleaved at a cleavage site, thereby releasing the inhibitory sequence from the sequence encoding the exogenous protein of interest whereby the exogenous protein of interest is capable of being expressed. In some embodiments sufficient complementarity is at least 30% complementarity. In other embodiments, sufficient complementarity is at least 90% complementarity.

According to some embodiments, the cleavage site is located within the binding site and the cleavage site is located between said inhibitory sequence and the sequence encoding the exogenous protein of interest.

In some embodiments, the binding site for the specific endogenous miRNA is of sufficient complementarity to a sequence within said specific endogenous miRNA, for said specific endogenous miRNA to direct cleavage of said exogenous RNA molecule at the cleavage site.

According to further embodiments, the specific endogenous miRNA is a cellular microRNA, a viral microRNA, or both. In some embodiments, the cellular microRNA is expressed only in neoplastic cells. In some embodiments, the viral microRNA is expressed by a virus selected from the group consisting of a double-stranded DNA virus, a single-stranded DNA virus, a double-stranded RNA virus, a double-stranded RNA virus, a single-stranded (plus-strand) virus, a single-stranded (minus-strand) virus and a retrovirus.

According to some embodiments, the exogenous protein of interest is a toxin. The toxin may be selected from a group consisting of: Ricin, Ricin A chain, Abrin, Abrin A chain, Diphtheria toxin A chain and modified forms thereof. In some embodiments, the toxin is selected from the group consisting of: alpha toxin, saporin, maize RIP, barley RIP, wheat RIP, corn RIP, rye RIP, flax RIP, Shiga toxin, Shiga-like RIP, momordin, thymidine kinase, pokeweed antiviral protein, gelonin, Pseudomonas exotoxin, Pseudomonas exotoxin A, Escherichia coli cytosine deaminase and modified forms thereof.

In additional embodiments, the inhibitory sequence may be located upstream from the cleavage site and the inhibitory sequence may directly or indirectly, reduce the efficiency of translation of said exogenous protein of interest from the exogenous RNA molecule.

In some embodiments, the inhibitory sequence comprises a plurality of initiation codons. In further embodiments, each of the initiation codons and the sequence encoding exogenous protein of interest are not in the same reading frame. In some embodiments, each of said initiation codons is consisting essentially of 5′-AUG-3′. In some embodiments, each of the initiation codons may be located within a Kozak consensus sequence.

According to further embodiments, the inhibitory sequence is capable of binding to a polypeptide, wherein the polypeptide, directly or indirectly may reduce the efficiency of translation of said exogenous protein of interest in the exogenous RNA molecule. The polypeptide may be a translation repressor protein, wherein the translation repressor protein is an endogenous translation repressor protein or is encoded by the one or more polynucleotides of the composition.

In some embodiments, the inhibitory sequence comprises an RNA localization signal for subcellular localization or an endogenous miRNA binding site.

According to some embodiments, the one or more polynucleotides of the composition may further comprises a polynucleotide sequence encoding a functional RNA that is capable of inhibiting the expression, directly or indirectly, of an endogenous exonuclease.

In some embodiments, the binding site for the specific endogenous miRNA is plurality of binding sites for the same or different endogenous miRNAs and wherein said cleavage site is a plurality of cleavage sites.

In some embodiments, the specific endogenous miRNA is selected from the group consisting of: hsv1-miR-H1, hsv1-miR-H2, hsv1-miR-H3, hsv1-miR-H4, hsv1-miR-H5, hsv1-miR-H6, hsv2-miR-I, hcmv-miR-UL22A, hcmv-miR-UL36, hcmv-miR-UL70, hcmv-miR-UL112, hcmv-miR-UL148D, hcmv-miR-US4, hcmv-miR-US5-1, hcmv-miR-US5-2, hcmv-miR-US25-1, hcmv-miR-US25-2, hcmv-miR-US33, kshv-miR-K12-1, kshv-miR-K12-2, kshv-miR-K12-3, kshv-miR-K12-4, kshv-miR-K12-5, kshv-miR-K12-6, kshv-miR-K12-7, kshv-miR-K12-8, kshv-miR-K12-9, kshv-miR-K12-10a, kshv-miR-K12-10b, kshv-miR-K12-11, kshv-miR-K12-12, ebv-miR-BART1, ebv-miR-BART2, ebv-miR-BART3, ebv-miR-BART4, ebv-miR-BART5, ebv-miR-BART6, ebv-miR-BART7, ebv-miR-BART8, ebv-miR-BART9, ebv-miR-BART10, ebv-miR-BART11, ebv-miR-BART12, ebv-miR-BART13, ebv-miR-BART14, ebv-miR-BART15, ebv-miR-BART16, ebv-miR-BART17, ebv-miR-BART18, ebv-miR-BART19, ebv-miR-BART20, ebv-miR-BHRF1-1, ebv-miR-BHRF1-2, ebv-miR-BHRF1-3, bkv-miR-B1, jcv-miR-J1, hiv1-miR-H1, hiv1-miR-N367, hiv1-miR-TAR, sv40-miR-S1, MCPyV-miR-M1, hsv1-miR-LAT, hsv1-miR-LAT-ICP34.5, hsv2-miR-II, hsv2-miR-III, hcmv-miR-UL23, hcmv-miR-UL36-1, hcmv-miR-UL54-1, hcmv-miR-UL70-1, hcmv-miR-UL22A-1, hcmv-miR-UL112-1, hcmv-miR-UL148D-1, hcmv-miR-US4-1, hcmv-miR-US24, hcmv-miR-US33-1, hcmv-RNAβ2.7, ebv-miR-BART1-1, ebv-miR-BART1-2, ebv-miR-BART1-3, ebv-miR-BHFR1, ebv-miR-BHFR2, ebv-miR-BHFR3, hiv1-miR-TAR-5p, hiv1-miR-TAR-p, hiv1-HAAmiRNA, hiv1-VmiRNA1, hiv1-VmiRNA2, hiv1-VmiRNA3, hiv1-VmiRNA4, mir-675, hiv1-VmiRNA5, hiv2-miR-TAR2-5p, hiv2-miR-TAR2-3p, mdv1-miR-M1, mdv1-miR-M2, mdv1-miR-M3, mdv1-miR-M4, mdv1-miR-M5, mdv1-miR-M6, mdv1-miR-M7, mdv1-miR-M8, mdv1-miR-M9, mdv1-miR-M10, mdv1-miR-M11, mdv1-miR-M12, mdv1-miR-M13, mdv2-miR-M14, mdv2-miR-M15, mdv2-miR-M16, mdv2-miR-M17, mdv2-miR-M18, mdv2-miR-M19, mdv2-miR-M20, mdv2-miR-M21, mdv2-miR-M22, mdv2-miR-M23, mdv2-miR-M24, mdv2-miR-M25, mdv2-miR-M26, mdv2-miR-M27, mdv2-miR-M28, mdv2-miR-M29, mdv2-miR-M30, mcmv-miR-M23-1, mcmv-miR-M23-2, mcmv-miR-M44-1, mcmv-miR-M55-1, mcmv-miR-M87-1, mcmv-miR-M95-1, mcmv-miR-m01-1, mcmv-miR-m01-2, mcmv-miR-m01-3, mcmv-miR-m01-4, mcmv-miR-m21-1, mcmv-miR-m22-1, mcmv-miR-m59-1, mcmv-miR-m59-2, mcmv-miR-m88-1, mcmv-miR-m107-1, mcmv-miR-m108-1, mcmv-miR-m108-2, rlcv-miR-rL1-1, rlcv-miR-rL1-2, rlcv-miR-rL1-3, rlcv-miR-rL1-4, rlcv-miR-rL1-5, rlcv-miR-rL1-6, rlcv-miR-rL1-7, rlcv-miR-rL1-8, rlcv-miR-rL1-9, rlcv-miR-rL1-10, rlcv-miR-rL1-11, rlcv-miR-rL1-12, rlcv-miR-rL1-13, rlcv-miR-rL1-14, rlcv-miR-rL1-15, rlcv-miR-rL1-16, rrv-miR-rR1-1, rrv-miR-rR1-2, rrv-miR-rR1-3, rrv-miR-rR1-4, rrv-miR-rR1-5, rrv-miR-rR1-6, rrv-miR-rR1-7, mghv-miR-M1-1, mghv-miR-M1-2, mghv-miR-M 1-3, mghv-miR-M1-4, mghv-miR-M1-5, mghv-miR-M1-6, mghv-miR-M1-7, mghv-miR-M1-8, mghv-miR-M1-9 and sv40-miR-S 1. The nomenclature and sequences thereof are as defined at the database http://www.mirbase.org/.

In some embodiments, the exogenous RNA molecule further comprises a stop codon that is located between the initiation codon and the start codon of said sequence encoding protein of interest, wherein said stop codon and said initiation codon are in the same reading frame and wherein said stop codon is selected from the group consisting of: 5′-UAA-3′,5′-UAG-3′ and 5′-UGA-3′.

In further embodiments, the inhibitory sequence is located upstream from the sequence encoding the exogenous protein of interest, wherein the inhibitory sequence is capable of forming a secondary structure having a folding free energy of lower than −30 kcal/mol, whereby said secondary structure is sufficient to block scanning ribosomes from reaching the start codon of said exogenous protein of interest.

In additional embodiments, the one or more polynucleotides of the composition comprise one or more DNA molecules, one or more RNA molecules or combinations thereof.

In further embodiments, the cell is selected from the group consisting of: human cell, animal cell, cultured cell and plant cell. In some embodiments, the cell is a neoplastic cell. In further embodiments, the cell is present in an organism.

In some embodiments, the composition is introduced into a cell. The cell may be a neoplastic cell and it may be present in an organism.

In some embodiments, there is further provided a diagnostic kit which comprises the composition.

In further embodiments, there is provided a pharmaceutical composition comprising the composition, which comprises the one or more polynucleotides, and one or more excipients.

In additional embodiments, there is provided a method for targeted killing of a target cell, which comprises the specific endogenous miRNA, the method comprising introducing into the target cell the composition which comprises the one or more polynucleotides.

According to some embodiments, there is provided a vector comprising a polynucleotide sequence encoding for an exogenous RNA molecule, wherein said exogenous RNA molecule comprises a sequence encoding for an exogenous protein of interest; an inhibitory sequence that is capable of inhibiting the expression of the exogenous protein of interest; and a binding site for a specific endogenous miRNA. The vector may be a viral vector. The vector may be a non-viral vector. In some embodiments, the binding site for the specific endogenous miRNA is of sufficient complementarity to a sequence within a specific endogenous miRNA for the specific endogenous miRNA to direct cleavage of said exogenous RNA molecule at the cleavage site, upon introducing the vector into a cell comprising said specific endogenous miRNA. In further embodiments, the cleavage site may be located within the binding site for the specific endogenous miRNA, and the cleavage site may be located between the inhibitory sequence and the sequence encoding the exogenous protein of interest. In further embodiments, the specific endogenous miRNA is a cellular microRNA, a viral microRNA, or both. The cellular microRNA may be expressed only in neoplastic cells. The viral microRNA may be expressed by a virus selected from the group consisting of a double-stranded DNA virus, a single-stranded DNA virus, a double-stranded RNA virus, a double-stranded RNA virus, a single-stranded (plus-strand) virus, a single-stranded (minus-strand) virus and a retrovirus.

According to further embodiments, the exogenous protein of interest is a toxin. The toxin may be selected from the group consisting of: Ricin, Ricin A chain, Abrin, Abrin A chain, Diphtheria toxin A chain and modified forms thereof. In further embodiments, the toxin may be selected from the group consisting of: alpha toxin, saporin, maize RIP, barley RIP, wheat RIP, corn RIP, rye RIP, flax RIP, Shiga toxin, Shiga-like RIP, momordin, thymidine kinase, pokeweed antiviral protein, gelonin, Pseudomonas exotoxin, Pseudomonas exotoxin A, Escherichia coli cytosine deaminase and modified forms thereof.

Objects and advantages of the present invention will be clear from the description that follows.

### BRIEF DESCRIPTION OF THE FIGURES

The following figures are offered by way of illustration and not by way of limitation.

FIG. 1 is a schematic drawing of a model for biogenesis and activity of microRNAs (miRNAs).

FIG. 2 is a schematic drawing illustrating, according to some embodiments, the activation of an exogenous RNA molecule by endogenous miRNA, such that the inhibitory sequence in the exogenous RNA molecule is located upstream from the cleavage site in the exogenous RNA molecule.

FIG. 3 is a schematic drawing illustrating, according to some embodiments, the activation of the exogenous RNA molecule by endogenous miRNA, such that the inhibitory sequence in the exogenous RNA molecule is located downstream from the cleavage site in the exogenous RNA molecule.

FIG. 4A is a schematic drawing showing an example, according to some embodiments, of inhibitory sequence in the exogenous RNA molecule, that is located upstream from the cleavage site and comprises an AUG that is not in the same reading frame with the sequence encoding the exogenous protein of interest.

FIG. 4B is a schematic drawing showing an example, according to some embodiments, of inhibitory sequence in the exogenous RNA molecule, that is located upstream from the cleavage site and comprises a Kozak consensus sequence (5′-ACCAUGG-3′-SEQ ID NO. 25) that is not in the same reading frame with the sequence encoding the exogenous protein of interest.

FIG. 4C is a schematic drawing showing an example, according to some embodiments, of inhibitory sequence in the exogenous RNA molecule, that is located upstream from the cleavage site and comprises 2 Kozak consensus sequence that are not in the same reading frame with the sequence encoding the exogenous protein of interest.

FIG. 5A is a schematic drawing showing an example, according to some embodiments, of inhibitory sequence in the exogenous RNA molecule, that is located upstream from the cleavage site and comprises an AUG and a downstream stop codon that are in the same reading frame.

FIG. 5B is a schematic drawing showing an example, according to some embodiments, of inhibitory sequence in the exogenous RNA molecule, that is located upstream from the cleavage site and comprises an AUG and a downstream sorting signal for subcellular localization or protein degradation signal.

FIG. 5C is a schematic drawing showing an example, according to some embodiments, of inhibitory sequence in the exogenous RNA molecule, that is located upstream from the cleavage site and comprises an AUG and a downstream sequence that encodes amino acids that are capable of inhibiting the biological function of the downstream exogenous protein of interest.

FIG. 5D is a schematic drawing showing an example, according to some embodiments, of inhibitory sequence in the exogenous RNA molecule, that is located upstream from the cleavage site and comprises an AUG, a downstream stop codon that is in the same reading frame with the AUG and a downstream intron, such that the exogenous RNA molecule is a target for nonsense-mediated decay (NMD).

FIG. 6A is a schematic drawing showing an example, according to some embodiments, for inhibitory sequence in the exogenous RNA molecule, that is located upstream from the cleavage site and comprises a Binding site for translation repressor.

FIG. 6B is a schematic drawing showing an example, according to some embodiments, of inhibitory sequence in the exogenous RNA molecule that is located upstream from the cleavage site and comprises an RNA localization signal for subcellular localization.

FIG. 6C is a schematic drawing showing an example, according to some embodiments, of inhibitory sequence in the exogenous RNA molecule that is located upstream from the cleavage site and comprises an RNA destabilizing element that is AU-rich element or endonuclease recognition site.

FIG. 6D is a schematic drawing showing an example, according to some embodiments, of inhibitory sequence in the exogenous RNA molecule that is located upstream from the cleavage site and comprises a secondary structure.

FIG. 7 is a schematic drawing showing an example, according to some embodiments, of the activation of the exogenous RNA molecule by endogenous miRNA, such that the inhibitory sequence creates a secondary structure that blocks translation and such that the cleavage by the miRNA creates an IRES (Internal ribosome entry site).

FIG. 8A is a schematic drawing showing an example, according to some embodiments, of additional structure that increases the efficiency of translation of the exogenous RNA molecule that is cleaved at the 5′ end, such that the additional structure is an IRES (Internal ribosome entry site).

FIG. 8B is a schematic drawing showing an example, according to some embodiments, of additional structure that increases the efficiency of translation of the exogenous RNA molecule that is cleaved at the 5′ end, such that the additional structure is a stem loop structure.

FIG. 8C is a schematic drawing showing an example, according to some embodiments, of additional structure that increases the efficiency of translation of the exogenous RNA molecule that is cleaved at the 5′ end, such that the additional structure is cytoplasmic polyadenylation element.

FIG. 8D is a schematic drawing showing an example, according to some embodiments, of additional structures that increase the efficiency of translation of the exogenous RNA molecule that is cleaved at the 5′ end, such that the additional structures are nucleotide sequences that are bind to each other and force the exogenous RNA molecule to form a circular structure particularly when the exogenous RNA molecule is cleaved at the cleavage site.

FIG. 9A is a schematic drawing showing an example, according to some embodiments, of additional structure that increases the efficiency of translation of the exogenous RNA molecule that is cleaved at the 5′ end, such that the additional structure is a polypeptide that is encoded from the composition of the invention, such that this polypeptide is capable of binding to the poly-A and to a sequence within the exogenous RNA molecule and thus forces the exogenous RNA molecule to form a circular structure particularly when the exogenous RNA molecule is cleaved at the cleavage site.

FIG. 9B is a schematic drawing showing an example, according to some embodiments, of additional structure that increases the efficiency of translation of the exogenous RNA molecule that is cleaved at the 5′ end, such that the additional structure is an additional RNA molecule that is encoded from the composition of the invention and is capable of binding to the exogenous RNA molecule and by this provide it with a CAP, when the exogenous RNA molecule is cleaved at the cleavage site.

FIG. 9C is a schematic drawing showing an example, according to some embodiments, of additional structure that reduces the efficiency of translation of the intact exogenous RNA molecule, such that the additional structure is a cis acting ribozyme that removes the CAP structure from the intact exogenous RNA molecule.

FIG. 10A is a schematic drawing showing the sequence of the very efficient cis-acting hammerhead ribozymes-snorbozyme (SEQ ID NO. 63) [15].

FIG. 10B is a schematic drawing showing the sequence of the very efficient cis-acting hammerhead ribozymes—N117 (SEQ ID NO. 64) [16].

FIG. 11A is a schematic drawing showing an example, according to some embodiments, of inhibitory sequence in the exogenous RNA molecule, that is located downstream from the cleavage site and comprises an intron, such that the exogenous RNA molecule is a target for nonsense-mediated decay (NMD).

FIG. 11B is a schematic drawing showing an example, according to some embodiments, of inhibitory sequence in the exogenous RNA molecule, that is located downstream from the cleavage site and comprises a Binding site for translation repressor.

FIG. 11C is a schematic drawing showing an example, according to some embodiments, for inhibitory sequence in the exogenous RNA molecule, that is located downstream from the cleavage site and comprises an RNA localization signal for subcellular localization.

FIG. 11D is a schematic drawing showing an example, according to some embodiments, for inhibitory sequence in the exogenous RNA molecule that is located downstream from the cleavage site and comprises an RNA destabilizing element that is AU-rich element or endonuclease recognition site.

FIG. 11E is a schematic drawing showing an example, according to some embodiments, of inhibitory sequence in the exogenous RNA molecule, that is located downstream from the cleavage site and comprises a secondary structure.

FIG. 12A is a schematic drawing showing an example, according to some embodiments, for inhibitory sequence in the exogenous RNA molecule, that is located downstream from the sequence encoding the exogenous protein of interest, such that the inhibitory sequence creates a secondary structure that blocks translation.

FIG. 12B is a schematic drawing showing an example, according to some embodiments, of additional structure that increases the efficiency of translation of the exogenous RNA molecule that is cleaved at the 3′ end, such that the additional structure is an IRES (Internal ribosome entry site).

FIG. 12C is a schematic drawing showing an example, according to some embodiments, of additional structure that increases the efficiency of translation of the exogenous RNA molecule that is cleaved at the 3′ end, such that the additional structure is a stem loop structure.

FIG. 12D is a schematic drawing showing an example, according to some embodiments, of additional structure that increases the efficiency of translation of the exogenous RNA molecule that is cleaved at the 3′ end, such that the additional structure is a cytoplasmic polyadenylation element.

FIG. 13A is a schematic drawing showing an example, according to some embodiments, of additional structures that increase the efficiency of translation of the exogenous RNA molecule that is cleaved at the 3′ end, such that the additional structures are nucleotide sequences that may bind to each other and force the exogenous RNA molecule to form a circular structure, when the exogenous RNA molecule is cleaved at the cleavage site.

FIG. 13B is a schematic drawing showing an example, according to some embodiments, of additional structure that increases the efficiency of translation of the exogenous RNA molecule that is cleaved at the 3′ end, such that the additional structure is a polypeptide that is encoded from the composition, wherein the polypeptide is capable of binding to the CAP and to a sequence within the exogenous RNA molecule and forces the exogenous RNA molecule to form a circular structure, in particular when the exogenous RNA molecule is cleaved at the cleavage site.

FIG. 13C is a schematic drawing showing an example, according to some embodiments, of additional structure that increases the efficiency of translation of the exogenous RNA molecule that is cleaved at the 3′ end, such that the additional structure is an additional RNA molecule that is encoded from the composition of the invention and is capable of binding to the exogenous RNA molecule and thus provide it a poly-A, in particular when the exogenous RNA molecule is cleaved at the cleavage site.

FIG. 13D is a schematic drawing showing an example, according to some embodiments, of additional structure that reduces the efficiency of translation of the intact exogenous RNA molecule, such that the additional structure is cis acting ribozyme that removes the poly-A from the intact exogenous RNA molecule.

FIG. 14A is a schematic drawing showing an example, according to some embodiments, of an exogenous RNA molecule that includes two binding sites for different endogenous miRNAs, such that the inhibitory sequence is located upstream from the cleavage site.

FIG. 14B is a schematic drawing showing an example, according to some embodiments, of an exogenous RNA molecule that includes two binding site for the same endogenous miRNA, such that the inhibitory sequence is located upstream from the cleavage site.

FIG. 14C is a schematic drawing showing an example, according to some embodiments, of an exogenous RNA molecule that includes two binding site for different endogenous miRNAs, such that the inhibitory sequence is located downstream from the cleavage site.

FIG. 14D is a schematic drawing showing an example, according to some embodiments, of an exogenous RNA molecule that comprises two binding site for the same endogenous miRNA, such that the inhibitory sequence is located downstream from the cleavage site.

FIG. 15A is a schematic drawing showing an example, according to some embodiments, of the exogenous RNA molecule having its inhibitory sequence located downstream from the sequence encoding the exogenous protein of interest, such that the exogenous RNA molecule further comprises an additional binding site for miRNA upstream from sequence encoding the exogenous protein of interest and an initiation codon upstream from the additional binding site such that the initiation codon is not in the same reading frame with the sequence encoding the exogenous protein of interest.

FIG. 15B is a schematic drawing showing an example, according to some embodiments, of the exogenous RNA molecule having its inhibitory sequence located downstream from the sequence encoding the exogenous protein of interest, and the exogenous RNA molecule further includes an additional binding site for miRNA, upstream from the sequence encoding the exogenous protein of interest and an initiation codon upstream from the additional binding site such that the initiation codon is not in the same reading frame with the sequence encoding the exogenous protein of interest and such that the exogenous RNA molecule further comprises a cis acting ribozyme at the 5′ end.

FIG. 15C is a schematic drawing showing an example, according to some embodiments, of an exogenous RNA molecule that includes the sequence encoding the exogenous protein of interest between two miRNA binding sites and further includes two inhibitory sequences one at the 5′ end and other at the 3′ end.

FIG. 15D is a schematic drawing showing an example, according to some embodiments, of an exogenous RNA molecule that includes the sequence encoding the exogenous protein of interest between two different miRNA binding sites and further comprises 2 inhibitory sequences, one at the 5′ end and other at the 3′ end.

FIG. 16A is a schematic drawing showing an example, according to some embodiments, of an inhibitory sequence in the exogenous RNA molecule, that is located downstream from the cleavage site and is capable of inhibiting the function of an RNA localization signal for subcellular localization.

FIG. 16B is a schematic drawing showing an example, according to some embodiments, of an inhibitory sequence in the exogenous RNA molecule, that is located upstream from the cleavage site and is capable of inhibiting the function of an RNA localization signal for subcellular localization.

FIG. 16C is a schematic drawing showing an example, according to some embodiments, of inhibitory sequence in the exogenous RNA molecule, that is located upstream from the cleavage site and comprises an AUG and a downstream sequence that encodes amino acids that are capable of inhibiting the function of the sorting signal for subcellular localization of the exogenous protein of interest.

FIG. 16D is a schematic drawing showing an example, according to some embodiments, of inhibitory sequence in the exogenous RNA molecule, that is located downstream from the miRNA binding site, such that the exogenous RNA molecule does not include a stop codon downstream from the start codon of the sequence encoding the exogenous protein of interest. The inhibitory sequence encodes an amino acid sequence that is capable of inhibiting the cleavage of a peptide sequence that is encoded upstream wherein the peptide sequence is capable of being cleaved by a protease in the target cell.

FIG. 17 is a schematic drawing illustrating the use, according to some embodiments, of the composition of the invention to kill Burkitt's lymphoma cancer cells, EBV-associated gastric carcinomas cancer cells and nasopharyngeal carcinoma cancer cells that comprise endogenous miR-BART1.

FIG. 18 is a schematic drawing illustrating an example, according for some embodiments, of using the composition of the invention to kill HIV-1 infected cells that comprise endogenous hiv1-miR-N367.

FIG. 19 is a schematic drawing showing an example, according to some embodiments, of using the composition of the invention to kill metastatic breast cancer cells that comprise endogenous miR-10b).

FIG. 20 is a schematic drawing showing an example, according to some embodiments, of using the composition of the invention to kill cells that comprise endogenous miR-LAT.

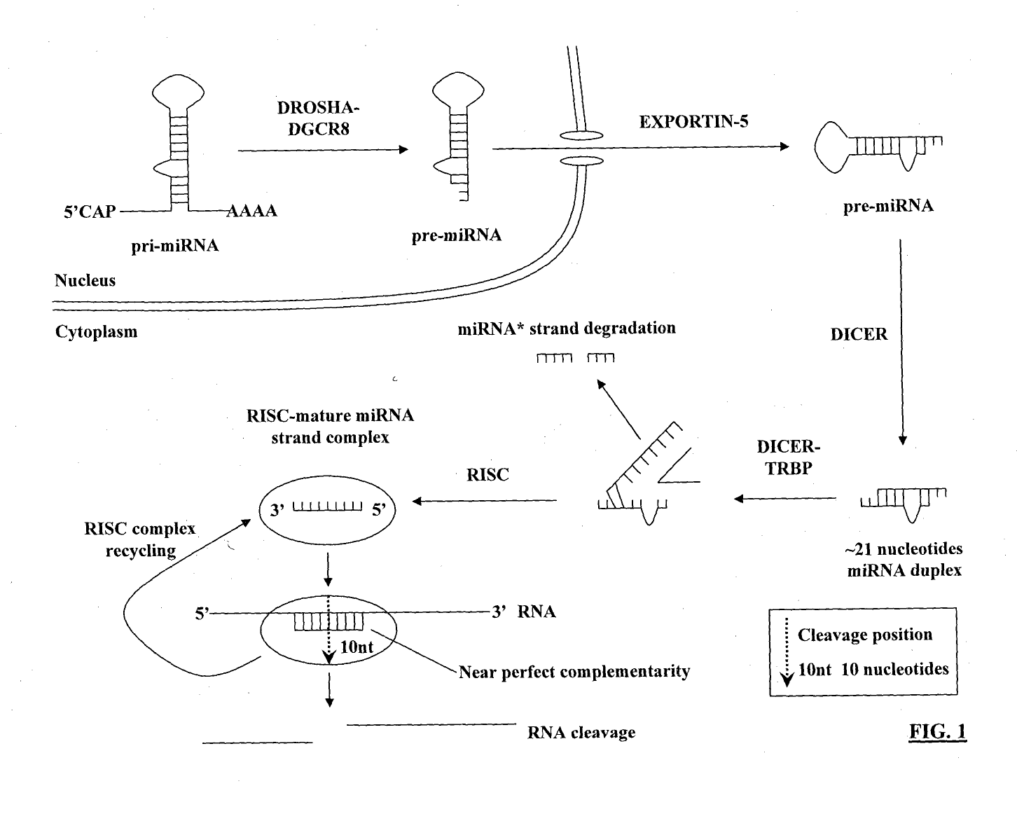

### DETAILED DESCRIPTION OF THE INVENTION

In the following detailed description of the invention when a reference term, such as: said, the, the last and the former; is used it refers to the exact term that is mentioned above (e.g. wherein said “The nucleic acid sequence” it refers to the nucleic acid sequence that is mentioned above and does not refer to the nucleotide sequence that is mentioned above). Furthermore, in the following detailed description of the invention each embodiment that refers to other embodiments is defined with them as a separate unit.

The following are terms which are used throughout the description and which should be understood in accordance with the various embodiments to mean as follows:

As referred to herein, the terms “polynucleotide molecules”, “oligonucleotide”, “polynucleotide”, “nucleic acid” and “nucleotide” sequences may interchangeably be used herein. The terms are directed to polymers of deoxyribonucleotides (DNA), ribonucleotides (RNA), and modified forms thereof in the form of a separate fragment or as a component of a larger construct, linear or branched, single stranded, double stranded, triple stranded, or hybrids thereof. The term also encompasses RNA/DNA hybrids. The polynucleotides may be, for example, sense and antisense oligonucleotide or polynucleotide sequences of DNA or RNA. The DNA or RNA molecules may be, for example, but are not limited to: complementary DNA (cDNA), genomic DNA, synthesized DNA, recombinant DNA, or a hybrid thereof or an RNA molecule such as, for example, mRNA, shRNA, siRNA, miRNA, and the like. Accordingly, as used herein, the terms “polynucleotide molecules”, “oligonucleotide”, “polynucleotide”, “nucleic acid” and “nucleotide” sequences are meant to refer to both DNA and RNA molecules. The terms further include oligonucleotides composed of naturally occurring bases, sugars, and covalent inter nucleoside linkages, as well as oligonucleotides having non-naturally occurring portions, which function similarly to respective naturally occurring portions.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

As referred to herein, the term “complementarity” is directed to base pairing between strands of nucleic acids. As known in the art, each strand of a nucleic acid may be complementary to another strand in that the base pairs between the strands are non-covalently connected via two or three hydrogen bonds. Two nucleotides on opposite complementary nucleic acid strands that are connected by hydrogen bonds are called a base pair. According to the Watson-Crick DNA base pairing, adenine (A) forms a base pair with thymine (T) and guanine (G) with cytosine (C). In RNA, thymine is replaced by uracil (U). The degree of complementarity between two strands of nucleic acid may vary, according to the number (or percentage) of nucleotides that form base pairs between the strands. For example, “100% complementarity” indicates that all the nucleotides in each strand form base pairs with the complement strand. For example, “95% complementarity” indicates that 95% of the nucleotides in each strand from base pair with the complement strand. The term sufficient complementarity may include any percentage of complementarity from about 30% to about 100%.

The term “construct”, as used herein refers to an artificially assembled or isolated nucleic acid molecule which may be comprises of one or more nucleic acid sequences, wherein the nucleic acid sequences may be coding sequences (that is, sequence which encodes for an end product), regulatory sequences, non-coding sequences, or any combination thereof. The term construct includes, for example, vectors but should not be seen as being limited thereto.

“Expression vector” refers to vectors that have the ability to incorporate and express heterologous nucleic acid fragments (such as DNA) in a foreign cell. In other words, an expression vector comprises nucleic acid sequences/fragments (such as DNA, mRNA, tRNA, rRNA), capable of being transcribed. Many viral, prokaryotic and eukaryotic expression vectors are known and/or commercially available. Selection of appropriate expression vectors is within the knowledge of those having skill in the art.

The terms “Upstream” and “Downstream”, as used herein refers to a relative position in a nucleotide sequence, such as, for example, a DNA sequence or an RNA sequence. As well known, a nucleotide sequence has a 5′ end and a 3′ end, so called for the carbons on the sugar (deoxyribose or ribose) ring of the nucleotide backbone. Hence, relative to the position on the nucleotide sequence, the term downstream relates to the region towards the 3′ end of the sequence. The term upstream relates to the region towards the 5′ end of the strand.

The terms “promoter element”, “promoter” or “promoter sequence” as used herein, refer to a nucleotide sequence that is generally located at the 5′ end (that is, precedes, located upstream) of the coding sequence and functions as a switch, activating the expression of a coding sequence. If the coding sequence is activated, it is said to be transcribed. Transcription generally involves the synthesis of an RNA molecule (such as, for example, a mRNA) from a coding sequence. The promoter, therefore, serves as a transcriptional regulatory element and also provides a site for initiation of transcription of the coding sequence into mRNA. Promoters may be derived in their entirety from a native source, or be composed of different elements derived from different promoters found in nature, or even comprise synthetic nucleotide segments. It is understood by those skilled in the art that different promoters may direct the expression of a gene in different tissues or cell types, or at different stages of development, or in response to different environmental conditions, or at various expression levels. Promoters which cause a gene to be expressed in most cell types at most times are commonly referred to as “constitutive promoters”. Promoters that derive gene expression in a specific tissue are called “tissue specific promoters”.

As referred to herein, the term “exogenous RNA molecule” is directed to a recombinant RNA molecule which is introduced to and/or expressed within a target cell. The exogenous RNA molecule may be intact (that is, a full-length molecule) or may be cleaved within the cell at one or more cleavage sites.

As referred to herein, the terms “protein of interest” and “exogenous protein of interest”, may interchangeably be used. The terms refer to a peptide sequence which is translated from an exogenous RNA molecule, within a cell. In some embodiments, the peptide sequence can be one or more separate proteins or a fusion protein.

As referred to herein, the terms “specific endogenous miRNA” and “specific miRNA” may interchangeably be used. The terms refer to an intracellular micro RNA (miRNA) molecule/sequence. The specific endogenous miRNA may be encoded by the genome of the cell (cellular miRNA), and/or from a foreign genome residing within the cell, such as, for example, from a virus residing within the cell (viral miRNA). The specific miRNA is present within the target cell prior to introduction/expression of an exogenous RNA molecule into the target cell.

The term “expression”, as used herein, refers to the production of a desired end-product molecule in a target cell. The end-product molecule may be, for example an RNA molecule; a peptide or a protein; and the like; or combinations thereof.

As referred to herein, the term, “Open Reading Frame” (“ORF”) is directed to a coding region which contains a start codon and a stop codon.

As referred to herein, the term “Kozak sequence” is well known in the art and is directed to a sequence on an mRNA molecule that is recognized by the ribosome as the translational start site. The terms “Kozak consensus sequence”, “Kozak consensus” or “Kozak sequence”, is a sequence which occurs on eukaryotic mRNA and has the consensus (gcc)gccRccAUGG (SEQ ID NO. 24), where R is a purine (adenine or guanine), three bases upstream of the start codon (AUG), which is followed by another ‘G’. In some embodiments, the Kozak sequence has the sequence RNNAUGG (SEQ ID NO. 83).

As used herein, the terms “introducing” and “transfection” may interchangeably be used and refer to the transfer of molecules, such as, for example, nucleic acids, polynucleotide molecules, vectors, and the like into a target cell(s), and more specifically into the interior of a membrane-enclosed space of a target cell(s). The molecules can be “introduced” into the target cell(s) by any means known to those of skill in the art, for example as taught by Sambrook et al. Molecular Cloning: A Laboratory Manual, Cold Spring Harbor Laboratory Press, New York (2001), the contents of which are incorporated by reference herein. Means of “introducing” molecules into a cell include, for example, but are not limited to: heat shock, calcium phosphate transfection, PEI transfection, electroporation, lipofection, transfection reagent(s), viral-mediated transfer, and the like, or combinations thereof. The transfection of the cell may be performed on any type of cell, of any origin, such as, for example, human cells, animal cells, plant cells, and the like. The cells may be isolated cells, tissue cultured cells, cell lines, cells present within an organism body, and the like.

The term “Kill” with respect to a cell/cell population is directed to include any type of manipulation that will lead to the death of that cell/cell population.

As referred to herein, the term “Treating a disease” or “treating a condition” is directed to administering a composition, which comprises at least one reagent (which may be, for example, one or more polynucleotide molecules, one or more expression vectors, one or more substance/ingredient, and the like), effective to ameliorate symptoms associated with a disease, to lessen the severity or cure the disease, or to prevent the disease from occurring. Administration may include any administration route.

The terms “Detection, “Diagnosis” refer to methods of detection of a disease, symptom, disorder, pathological or normal condition; classifying a disease, symptom, disorder, pathological condition; determining a severity of a disease, symptom, disorder, pathological condition; monitoring disease, symptom, disorder, pathological condition progression; forecasting an outcome and/or prospects of recovery thereof.

### 1. Basic Structure of Compositions of the Invention

According to some embodiments, there are provided composition for expressing an exogenous protein of interest only in a cell which comprises a specific endogenous miRNA. The endogenous miRNA may a cellular miRNA, a viral miRNA and/or any type of miRNA which is present in the cell. The exogenous protein of interest may be any type of peptide or protein, such as, for example, a toxin.

According to some embodiments, the composition of the invention may comprise one or more polynucleotide molecules, such as, for example, DNA molecules, RNA molecules, or both.

In some embodiments, the composition comprises or encodes for an exogenous RNA molecule which is an RNA molecule that includes at least the following sequences:
a) a sequence encoding for the exogenous protein of interest;
b) an inhibitory sequence that is capable of inhibiting the expression of the exogenous protein of interest; and
c) a binding site that is designed to be of sufficient complementarity to the mature miRNA strand of the specific endogenous miRNA for the specific endogenous miRNA to direct cleavage of the exogenous RNA molecule at a cleavage site. The cleavage site is designed to be located between the inhibitory sequence and the sequence encoding the exogenous protein of interest.

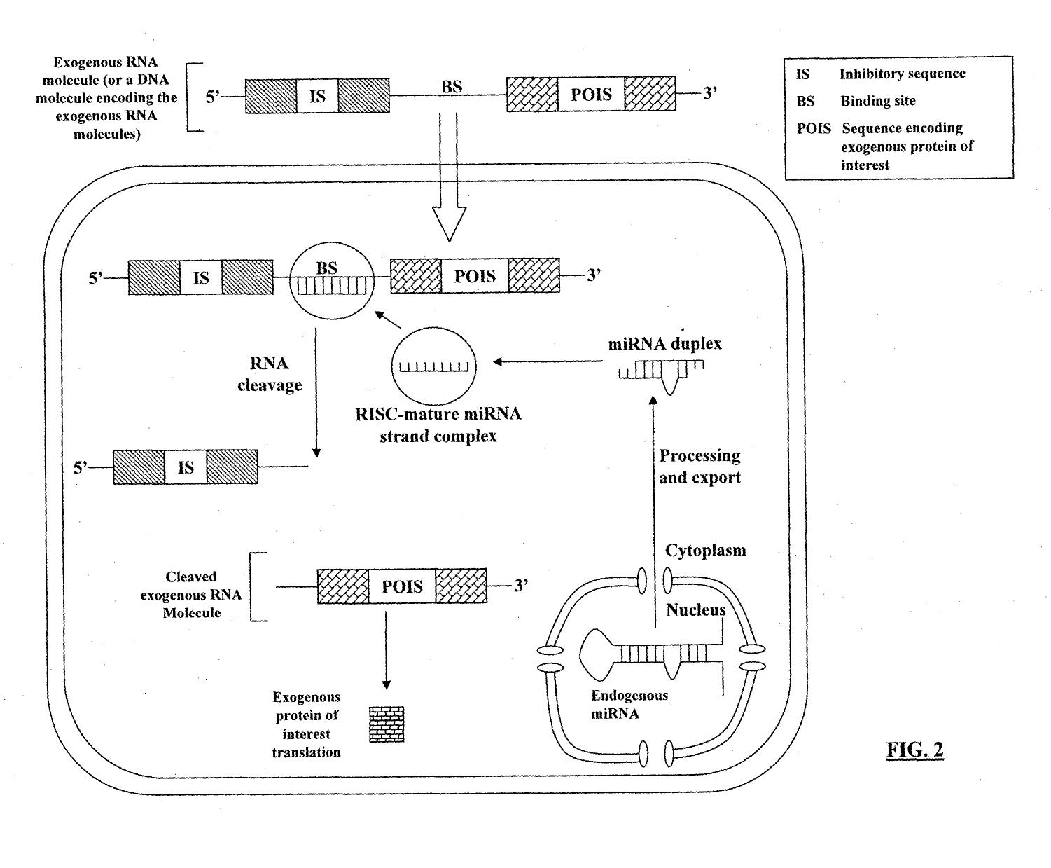

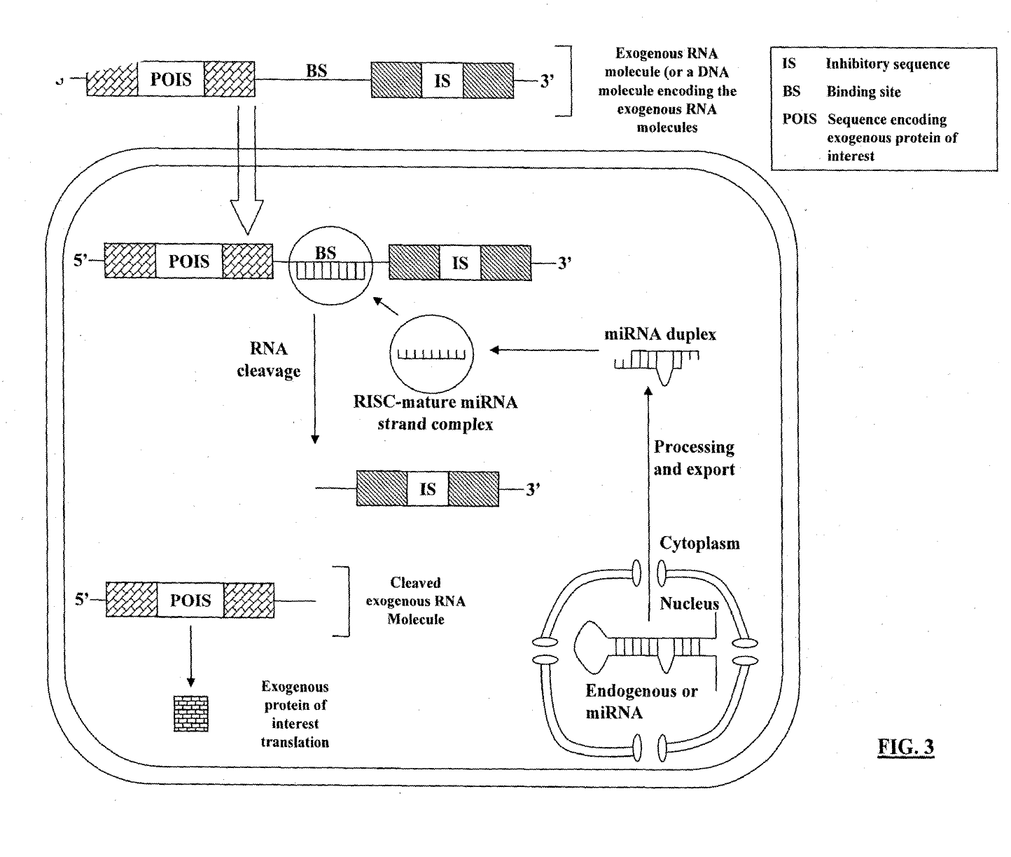

Thus, only in the presence of the specific endogenous miRNA in the cell, the exogenous RNA molecule is cleaved by the specific endogenous miRNA at the cleavage site and the inhibitory sequence is detached from the sequence encoding the exogenous protein of interest and the exogenous protein of interest is capable of being expressed. This is illustrated, for example, in FIGS. 2 and 3.

According to some embodiments, choosing the specific endogenous miRNA may be related and/or determined according to its expression within a specific cell type, which is the target cell. Hence, choosing a specific endogenous miRNA expressed in a specific cell type may thus provide a mechanism for the targeted expression of the exogenous protein of interest in a selected cell type (the target cell). The specific cells may be selected from, for example, but not limited to: cells infected with viral or other infectious agents; benign or malignant cells, cells expressing components of the immune system. Specificity may be achieved by modification of the binding site of the exogenous RNA molecule of the composition to be of sufficient complementarity to the mature miRNA strand of the specific endogenous miRNA for the specific endogenous miRNA to direct cleavage of the exogenous RNA molecule in the target cell.

It is known in the art that mRNAs without cap or poly A tail are still capable of translating proteins. In mammal cells, an addition of a cap increases the translation of an mRNA by 35-50 fold and an addition of a poly(A) tail increases the translation of an mRNA by 114-155-fold [6]. The poly(A) tail in mammal cells increases the functional mRNA half-life only by 2.6-fold and the cap increases the functional mRNA half-life only by 1.7-fold [6].

It is further known in the art that some proteins may exert a biological effect on a cell even at a concentration of one protein per cell. It has been reported, for example, that a single protein of Ricin or Abrin reaching the cytosol of a cell can kill the cell [3, 4]. In addition, a single protein of Diphtheria toxin fragment A (DTA) introduced into a cell can kill the cell [5]. In some embodiments, the exogenous protein of interest may be any protein or peptide, such as, for example, but not limited to Ricin, Abrin, Diphtheria toxin, and the like or combinations thereof.

According to some embodiments, the exogenous protein of interest may be a polypeptide which is a fusion of two proteins, that may have a cleavage site there between, allowing the separation of the two proteins within the cell. For example, the exogenous protein of interest may be a fusion protein of Ricin and DTA, whereby cleavage of the fusion protein by, for example, by a specific protease, can result in the formation of separate DTA and Ricin proteins in the cell. In some embodiments, the exogenous protein of interest may be two separate proteins that may be expressed by the composition. For example, the exogenous RNA of interest may encode for two separate exogenous proteins of interest, such as, for example, Ricin and DTA.

### 2. Structure of the Exogenous RNA Molecule Having an Inhibitory Sequence Located Upstream from the Cleavage Site

2.1. Structure of the Inhibitory Sequence that is Located Upstream from the Cleavage Site

According to some embodiments, the inhibitory sequence in the exogenous RNA molecule may be located upstream or downstream from the cleavage site. This section describes the structure of the inhibitory sequence that is located upstream from the cleavage site in the exogenous RNA molecule. This is illustrated, for example in FIG. 2.

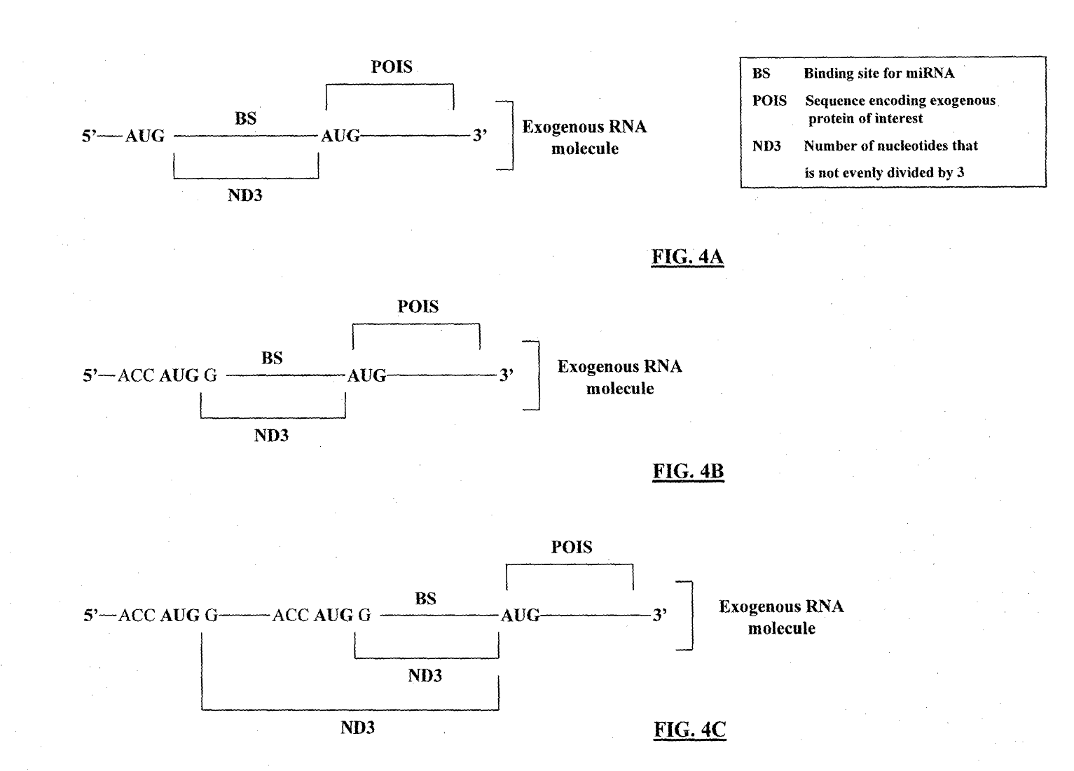

According to some embodiments, the inhibitory sequence that is located upstream from the cleavage site may be, for example, an initiation codon. The initiation codon and the sequence encoding the exogenous protein of interest are not in the same reading frame, such that the initiation codon may cause a frameshift mutation to the exogenous protein of interest, the coding sequence of which is located downstream. This is illustrated, for example, in FIG. 4A. In one embodiment, the initiation codon may be located within a Kozak consensus sequence. In addition, a modified. Kozak consensus sequences that maintain the ability to function as initiator of translation may be also be used. For example, see FIG. 4B. In some embodiments, the Kozak consensus sequence in human is 5′-ACCAUGG-3′ (SEQ ID NO. 25) and the initiation codon is 5′-AUG-3′.

In some embodiments, the initiation codon may be located within or may have one or more TISU motifs. A TISU (Translation Initiator of Short 5′UTR) motif is distinguished from a Kozak consensus in its unique ability to direct efficient and accurate translation initiation from mRNAs with a very short 5′UTR. [38].

In another embodiment, the inhibitory sequence that is located upstream from the cleavage site may have a plurality of initiation codons, such that each of the initiation codons and the sequence encoding the exogenous protein of interest are not in the same reading frame. The initiation codons may cause a frameshift mutation to the exogenous protein of interest, the encoding sequence of which is located downstream. Additionally, each of the initiation codons may be located within a Kozak consensus sequence or a modified Kozak consensus sequences that maintain the ability to function as initiator of translation. For example, see FIG. 4C.

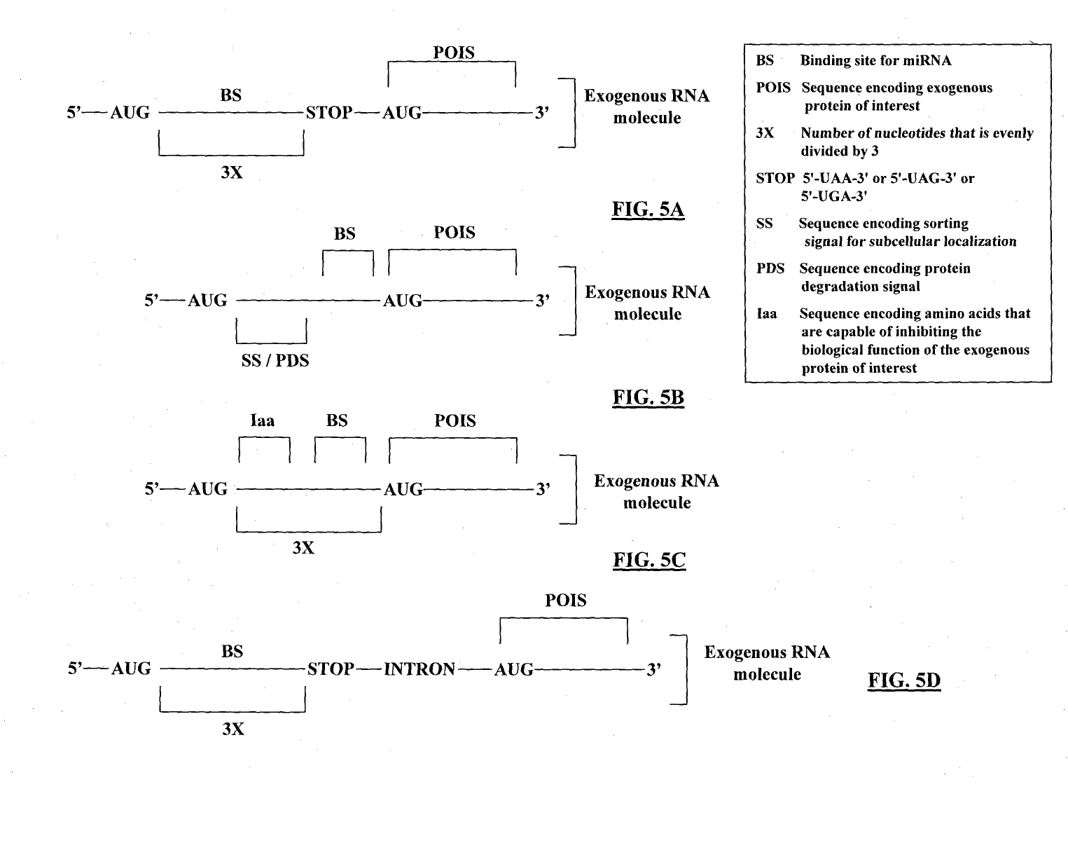

In another embodiment, the inhibitory sequence that is located upstream from the cleavage site may comprise an initiation codon. The exogenous RNA molecule may further comprise a stop codon between the initiation codon and the start codon of the sequence encoding the exogenous protein of interest, wherein the stop codon and the initiation codon are in the same reading frame. In such embodiment, an upstream open reading frame (uORF) is created that may reduce the efficiency of translation of the downstream sequence encoding the exogenous protein of interest. For example, see FIG. 5A. In some embodiments, the stop codon may be, for example, 5′-UAA-3′ or 5′-UAG-3′ or 5′-UGA-3′.

In some embodiments, strong stems and loops may be located downstream to upstream ORF(s) at a location that is upstream or downstream to the target sequence for the miRNA (cleavage site). The creation of such stems and loops may aid in conditions, wherein despite having reached a stop codon, the small subunit of the ribosome does not detach from the mRNA continue to scan the mRNA. The small subunit of the ribosome is not capable of opening strong RNA secondary structures. Additionally, when these stems and loops are located downstream to the target sequence they may also block the degradation of the cleaved mRNA which may be performed, for example, by XRN1 exorinonuclease.

In another embodiment, the inhibitory sequence that is located upstream from the cleavage site may comprise an initiation codon and a nucleotide sequence which encodes for a sorting signal for subcellular localization. The nucleotide sequence may be located downstream from the initiation codon and the nucleotide sequence and the initiation codon are in the same reading frame. In some embodiments, the subcellular localization, of the exogenous protein of interest, which is dictated by the sorting signal, may inhibit the biological function of the protein of interest. The sorting signal for the subcellular localization may be, for example, but is not limited to: a sorting signal for mitochondria, sorting signal for nucleus, sorting signal for endosome, sorting signal for lysosome, sorting signal for peroxisome, sorting signal for ER, and the like. The sorting signal for the subcellular localization may be, for example, a peroxisomal targeting signal 2 [(R/K)(L/V/I)X₅(Q/H)(L/A)] (SEQ ID NO. 26) or H₂N—RLRVLSGHL (SEQ ID NO. 27) (of human alkyl dihydroxyacetonephosphate synthase) [28]. This is shown, for example, in FIG. 5B.

In another embodiment of the invention, the inhibitory sequence that is located upstream from the cleavage site may comprise an initiation codon and a nucleotide sequence which encodes for a protein degradation signal. The nucleotide sequence is located downstream from the initiation codon such that the nucleotide sequence and the initiation codon are in the same reading frame. The protein degradation signal may be, for example, but is not limited to a ubiquitin degradation signal. For example, see FIG. 5B.

In another embodiment of the invention, the inhibitory sequence that is located upstream from the cleavage site may be designed to include an initiation codon and a nucleotide sequence downstream from the initiation codon that is in the same reading frame with the initiation codon and with the sequence encoding the exogenous protein of interest, such that when the amino acid sequence, which is encoded by the nucleotide sequence, is fused to the exogenous protein of interest the biological function of the exogenous protein of interest is inhibited. For example, see FIG. 5C.

In another embodiment of the invention, the inhibitory sequence that is located upstream from the cleavage site may comprise an initiation codon and the exogenous RNA molecule may further comprise a stop codon downstream from the initiation codon, such that the stop codon and the initiation codon are in the same reading frame. In addition the exogenous RNA molecule may further comprise an intron downstream from the stop codon, such that the exogenous RNA molecule is a target for nonsense-mediated decay (NMD) that may degrade the exogenous RNA molecule [29]. For example, see FIG. 5D.

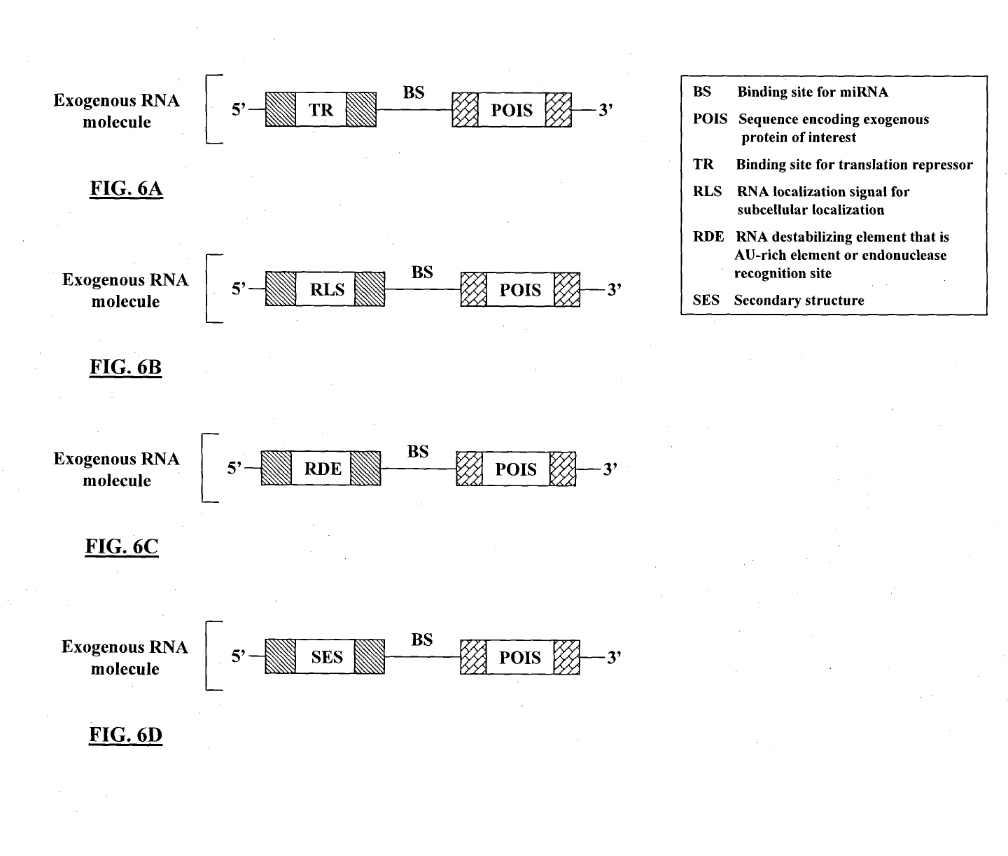

In another embodiment, the inhibitory sequence that is located upstream from the cleavage may comprise a sequence that is capable of binding to a translation repressor protein. In some embodiments, the translation repressor protein is an endogenous translation repressor protein. In some embodiments, the translation repressor protein may be encoded from the composition. The translation repressor protein, directly or indirectly may reduces the efficiency of translation of the exogenous protein of interest [24]. For example, a sequence that is capable of binding to a translation repressor protein includes; but is not limited to a sequence that binds the SMAUG repressor protein (5′-UGGAGCAGAGGCUCUGGCAGCUUUUGCAGCG-3′) (SEQ ID NO. 28) [25]. For example, see FIG. 6A.

In another embodiment, the inhibitory sequence that is located upstream from the cleavage site may comprise an RNA localization signal for subcellular localization (including, for example, co-translational import) or an endogenous miRNA binding site, such that the subcellular localization of the exogenous RNA molecule may inhibit the translation of the exogenous protein of interest and may decrease the exogenous RNA molecule half-life. The RNA localization signal may be, for example, but is not limited to RNA localization signal for: myelinating periphery, myelin compartment, mitochondria, leading edge of the lamella, Perinuclear cytoplasm [22], or the like. For example, the RNA localization signal may be an RNA localization signal for myelinating periphery 5′-GCCAAGGAGCCAGAGA GCAUG-3′ (SEQ ID NO. 29) or 5′-GCCAAGGAGCC-3′ (SEQ ID NO. 30) [27]. For example, see FIG. 6B.

In another embodiment, the inhibitory sequence that is located upstream from the cleavage site may comprise an RNA destabilizing element that may stimulate the degradation of the exogenous RNA molecule. The RNA destabilizing element may be, for example an AU-rich element (ARE), an endonuclease recognition site, or the like. The AU-rich element may be, for example, AU-rich elements that are at least about 35 nucleotides long. For example, the AU-rich elements may be 5′-AUUUA-3′ (SEQ ID NO. 31), 5′-UUAUUUA(U/A)(U/A)-3′ (SEQ ID NO. 32) or 5′-AUUU-3′ (SEQ ID NO. 33) [26]. For example, see FIG. 6C.

In another embodiment, the inhibitory sequence that is located upstream from the cleavage site may comprise a sequence that is capable of forming a secondary structure that may reduce the efficiency of translation of the downstream exogenous protein of interest. In some embodiments, the folding free energy of the secondary structure may be lower than −30 kcal/mol (for example, −50 kcal/mol, −80 kcal/mol) and thus the secondary structure is sufficient to block scanning ribosomes from reaching the start codon of the downstream region encoding the exogenous protein of interest. For example, see FIG. 6D.

In further embodiments the inhibitory sequence that is located upstream from the cleavage site may comprise a nucleotide sequence located immediately upstream from the cleavage site, wherein the nucleotide sequence is capable of binding to the nucleotide sequence that is located immediately downstream from the cleavage site for the formation of a secondary structure, such that the secondary structure, directly or indirectly, may reduce the efficiency of translation of the downstream exogenous protein of interest.

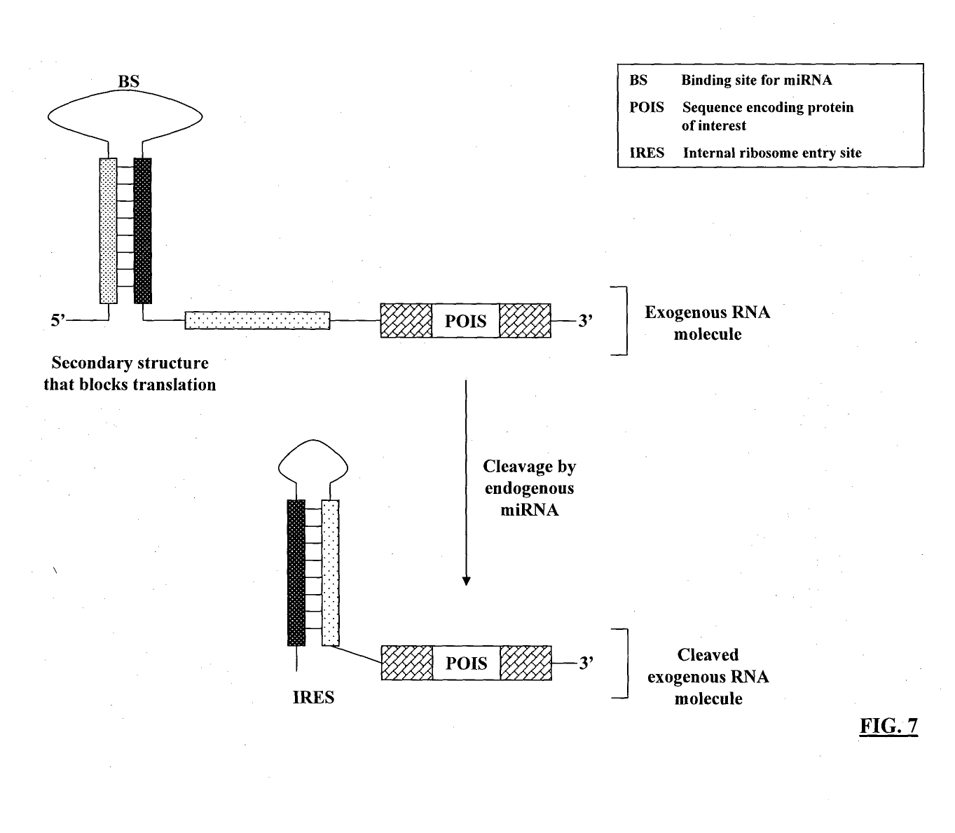

The folding free energy of the secondary structure may be lower than −30 kcal/mol (for example, −50 kcal/mol, −80 kcal/mol) and thus this secondary structure may be sufficient to block scanning ribosomes from reaching the start codon of the exogenous protein of interest. In another embodiment, the cleavage site may be located within a single stranded region or within a loop region in the secondary structure, such that the single stranded region or the loop region may be, for example, but is not limited to a region that is at least about 15 nucleotides long. In another embodiment, the exogenous RNA molecule may further comprise an internal ribosome entry site (IRES) sequence downstream from the cleavage site and upstream from the sequence encoding the exogenous protein of interest, such that the IRES sequence is more functional within the cleaved exogenous RNA molecule than within the intact exogenous RNA molecule. In another embodiment, at least part of the IRES sequence may be located within the nucleotide sequence that is located immediately downstream from the cleavage site. For example, see FIG. 7.

The IRES sequence may be selected from, for example, but is not limited to a picornavirus IRES, a foot-and-mouth disease virus IRES, an encephalomyocarditis virus IRES, a hepatitis A virus IRES, a hepatitis C virus IRES, a human rhinovirus IRES, a poliovirus IRES, a swine vesicular disease virus IRES, a turnip mosaic potyvirus IRES, a human fibroblast growth factor 2 mRNA IRES, a pestivirus IRES, a Leishmania RNA virus IRES, a Moloney murine leukemia virus IRES a human rhinovirus 14 IRES, anaphthovirus IRES, a human immunoglobulin heavy chain binding protein mRNA IRES, a Drosophila Antennapedia mRNA IRES, a human fibroblast growth factor 2 mRNA IRES, a hepatitis G virus IRES, a tobamovirus IRES, a vascular endothelial growth factor mRNA IRES, a Coxsackie B group virus IRES, a c-myc protooncogene mRNA IRES, a human MYT2 mRNA IRES, a human parechovirus type 1 virus IRES, a human parechovirus type 2 virus IRES, a eukaryotic initiation factor 4GI mRNA IRES, a Plautia stali intestine virus IRES, a Theiler's murine encephalomyelitis virus IRES, a bovine enterovirus IRES, a connexin 43 mRNA IRES, a homeodomain protein Gtx mRNA IRES, an AML1 transcription factor mRNA IRES, an NF-kappa B repressing factor mRNA IRES, an X-linked inhibitor of apoptosis mRNA IRES, a cricket paralysis virus RNA IRES, a p58(PITSLRE) protein kinase mRNA IRES, an ornithine decarboxylase mRNA IRES, a connexin-32 mRNA IRES, a bovine viral diarrhea virus IRES, an insulin-like growth factor I receptor mRNA IRES, a human immunodeficiency virus type 1 gag gene IRES, a classical swine fever virus IRES, a Kaposi's sarcoma-associated herpes virus IRES, a short IRES selected from a library of random oligonucleotides, a Jembrana disease virus IRES, an apoptotic protease-activating factor 1 mRNA IRES, a Rhopalosiphum padi virus IRES, a cationic amino acid transporter mRNA IRES, a human insulin-like growth factor II leader 2 mRNA IRES, a giardiavirus IRES, a Smad5 mRNA IRES, a porcine teschovirus-1 talfan IRES, a Drosophila Hairless mRNA IRES, an hSNM1 mRNA IRES, a Cbfa1/Runx2 mRNA IRES, an Epstein-Barr virus IRES, a hibiscus chlorotic ringspot virus IRES, a rat pituitary vasopressin V1b receptor mRNA IRES or a human hsp70 mRNA IRES.

2.2. Additional Structures that May Increase the Efficiency of Translation of the Exogenous RNA Molecule, which is Cleaved at the 5′ End

This section details additional embodiments of structures that may increase the efficiency of translation of the cleaved exogenous RNA molecule, wherein the cleaved exogenous RNA molecule is cleaved at the cleavage site at the 5′ end.

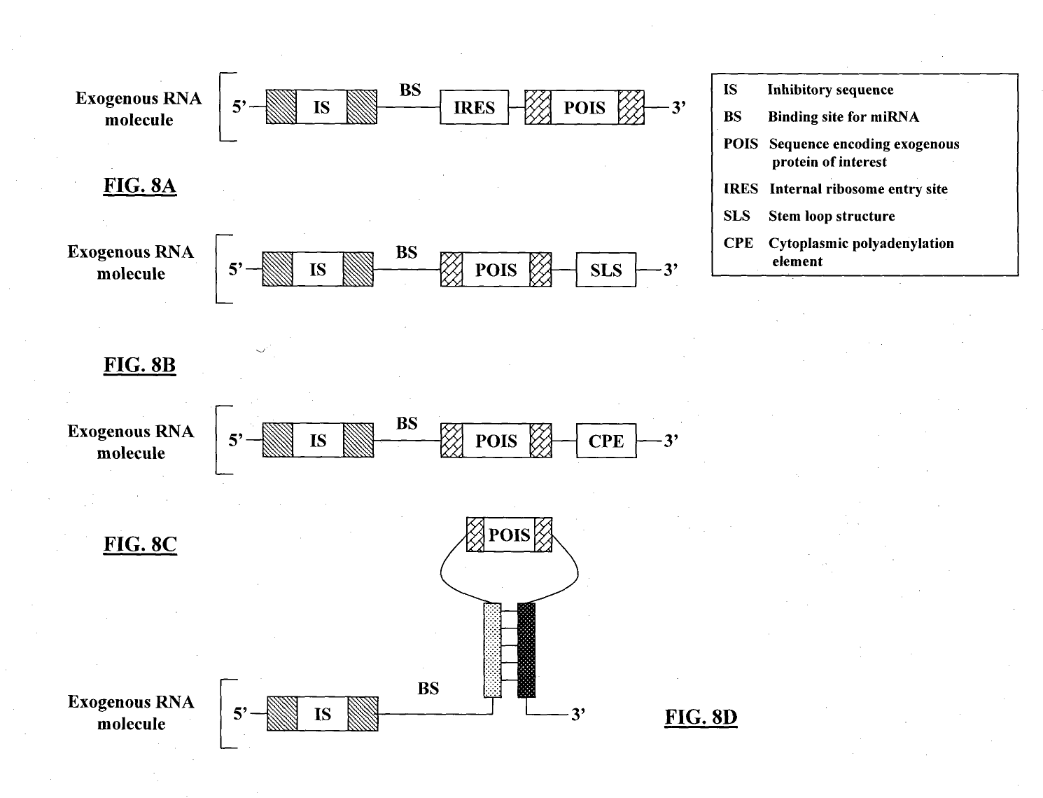

According to some embodiments, the exogenous RNA molecule may comprise a sequence that comprises a unique internal ribosome entry site (IRES) sequence immediately upstream from the sequence encoding the exogenous protein of interest, such that the unique IRES sequence increases the efficiency of translation of the exogenous protein of interest in the cleaved exogenous RNA molecule. For example, see FIG. 8A.

In another embodiment, the exogenous RNA molecule may comprise a unique nucleotide sequence immediately downstream from the sequence encoding the exogenous protein of interest, such that the unique nucleotide sequence comprises a unique stem loop structure and such that the unique stem loop structure, directly or indirectly, may increase the efficiency of translation of the exogenous protein of interest and the exogenous RNA molecule half-life in the cleaved exogenous RNA molecule. The unique stem loop structure may be, for example, but is not limited to a conserved stem loop structure of the human histone gene 3′-UTR or a functional derivative thereof. The conserved stem loop structure of the human histone gene may be, for example, 3′-UTR is 5′-GGCUCUUUUCAGAGCC-3′ (SEQ ID NO. 34). For example, see FIG. 8B.

In additional embodiments, the exogenous RNA molecule may comprise a unique nucleotide sequence immediately downstream from the sequence encoding the exogenous protein of interest, such that the unique nucleotide sequence comprises a cytoplasmic polyadenylation element that, directly or indirectly, may increase the efficiency of translation of the exogenous protein of interest and the exogenous RNA molecule half-life in the cleaved exogenous RNA molecule. The cytoplasmic polyadenylation element may be, for example, but is not limited to: 5′-UUUUAU-3′ (SEQ ID NO. 35), 5′-UUUUUAU-3′ (SEQ ID NO. 36), 5′-UUUUAAU-3′ (SEQ ID NO. 37), 5′-UUUUUUAUU-3′ (SEQ ID NO. 38), 5′-UUUUAUU-3′ (SEQ ID NO. 39) or 5′-UUUUUAUAAAG-3′ (SEQ ID NO. 40) [23].

In some embodiments, the composition of the invention may further comprise a polynucleotide sequence that encodes a human cytoplasmic polyadenylation element binding protein (hCPEB), or a homologue thereof for expressing hCPEB in any cell. For example, see FIG. 8C.

In further embodiments, the exogenous RNA molecule may comprise a unique nucleotide sequence that is located downstream from the cleavage site and upstream from the sequence encoding the exogenous protein of interest, such that the unique nucleotide sequence is capable of binding to a sequence that is located downstream from the sequence encoding for the exogenous protein of interest. In this embodiment, the cleaved exogenous RNA molecule may create a circular structure that may increase the efficiency of translation of the exogenous protein of interest in the cleaved exogenous RNA molecule. For example, see FIG. 8D.

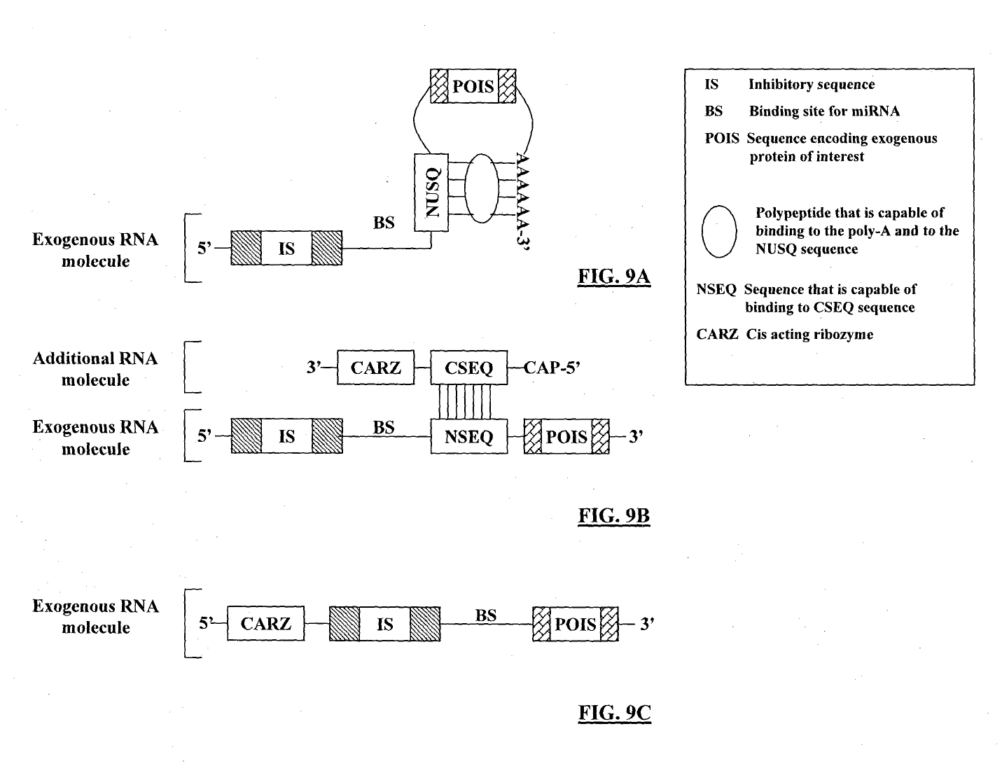

In another embodiment, the exogenous RNA molecule may comprise a unique nucleotide sequence that is located downstream from the cleavage site and upstream from the sequence encoding the exogenous protein of interest. The unique nucleotide sequence may be capable of binding to a unique polypeptide that is, directly or indirectly, capable of binding to the poly(A) tail in the cleaved exogenous RNA molecule. The unique polypeptide may also be encoded from the composition of the invention. In this embodiment, the unique polypeptide and the cleaved exogenous RNA molecule may create a circular structure that may increase the efficiency of translation of the exogenous protein of interest in the cleaved exogenous RNA molecule. For example, see FIG. 9A.

In another embodiment, the composition of the invention may further comprise an additional polynucleotide sequence, which encodes for an additional RNA molecule that comprises at the 5′ end a unique nucleotide sequence that is capable of binding to a sequence that is located downstream from the cleavage site and upstream from the sequence encoding the exogenous protein of interest. The expression of the additional polynucleotide sequence may be driven by, for example, polymerase II based promoter. In some embodiments, the composition of the invention may further comprise a cleaving component(s) that is capable of affecting the cleavage, directly or indirectly, of the additional RNA molecule at a position that is located downstream from the unique nucleotide sequence. The cleaving component(s) may be, for example:
(a) a unique nucleic acid sequence that is located within the additional RNA molecule, such that the unique nucleic acid sequence may be, but is not limited to: endonuclease recognition site, endogenous miRNA binding site, cis acting ribozyme, palindromic termination element or miRNA sequence; or
(b) a unique inhibitory RNA that is encoded from the composition of the invention, such that the unique inhibitory RNA may be, but is not limited to: microRNA (miRNA), lariat-form RNA, short-hairpin RNA (shRNA), siRNA expression domain, antisense RNA, double-stranded RNA (dsRNA), small-interfering RNA (siRNA) or ribozyme.

In this embodiment the additional RNA molecule may be capable of binding to the cleaved exogenous RNA molecule and provide it with a CAP structure that may increase the efficiency of translation of the exogenous protein of interest in the cleaved exogenous RNA molecule. For example, see FIG. 9B.

In some embodiments, a vpg recognition sequence may be introduced, such that upon cleave, the 5′ cleaved end contains a vpg recognition sequence. To the vpg recognition sequence a VPG protein may bind, thereby replacing the CAP. The vpg protein may be encoded by the composition of the invention or by the first ORF of the inhibitory sequence.

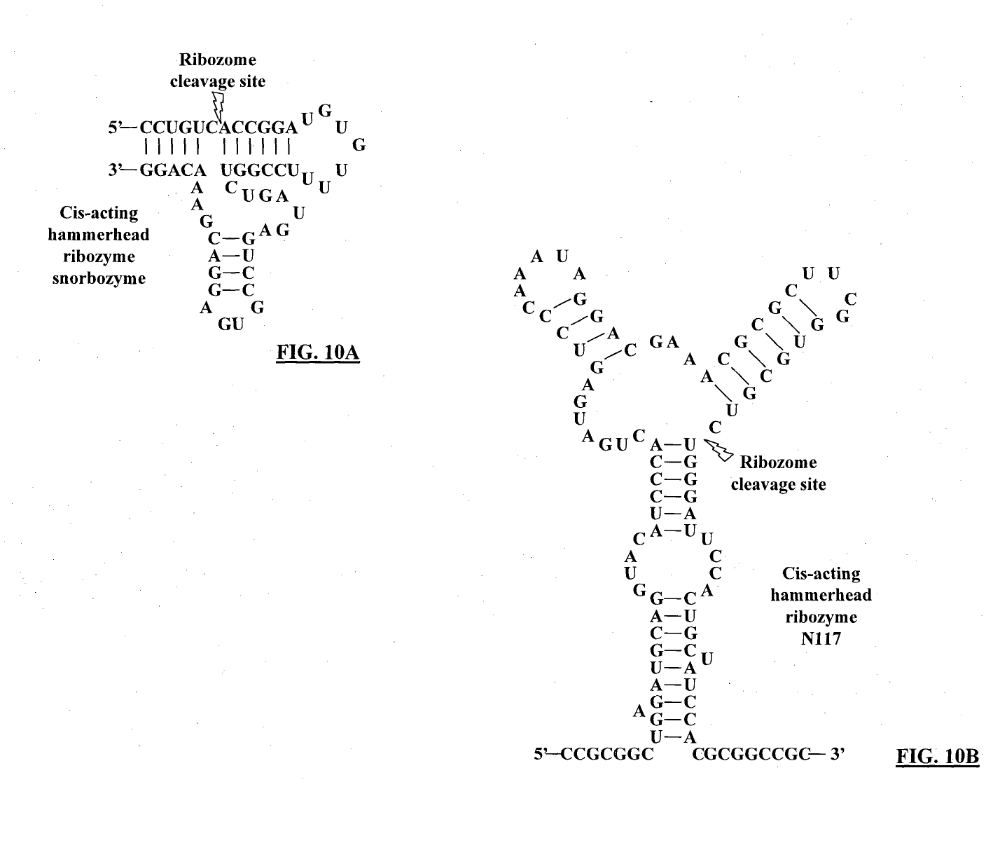

In some embodiments, and without wishing to be bound to theory or mechanism, the use of cis acting ribozyme is advantageous because the additional RNA molecule that comprises it may be cleaved by itself [15]. The cis acting ribozyme may be, for example, but is not limited to the very efficient cis-acting hammerhead ribozymes: snorbozyme [15] or N117 [16]. See FIG. 10A, 10B.

In another embodiment, the exogenous RNA molecule may further comprise a nucleotide sequence immediately upstream from the sequence encoding the exogenous protein of interest, such that the nucleotide sequence includes a stem loop structure that may reduce the degradation of the cleaved exogenous RNA molecule. In one embodiment, the stem loop structure is a conserved stem loop structure of human histone gene 3′-UTR (5′-GGCUCUUUUCAGAGCC-3′-SEQ ID NO. 34) or a functional derivative thereof.

2.3. Additional Structures that May Reduce the Efficiency of Translation of the Intact Exogenous RNA Molecule

This section describes various embodiments for additional structures, wherein these additional structures may reduce the efficiency of translation of the intact exogenous RNA molecule (that is, before the exogenous RNA molecule is cleaved).

In some embodiments, the composition may comprise a particular cleaving component(s) that is capable of effecting the cleavage, directly or indirectly, of the exogenous RNA molecule at a position that is located upstream from the inhibitory sequence, wherein the inhibitory sequence is located upstream from the cleavage site. The particular cleaving component(s) may be, for example:
(a) a particular nucleic acid sequence that is located within the exogenous RNA molecule, such that the particular nucleic acid sequence may be, for example, but is not limited to: endonuclease recognition site, endogenous miRNA binding site, cis acting ribozyme or miRNA sequence; or
(b) a particular inhibitory RNA that is encoded from the composition of the invention, such that the particular inhibitory RNA may be, for example, but is not limited to: microRNA (miRNA), lariat-form RNA, short-hairpin RNA (shRNA), siRNA expression domain, antisense RNA, double-stranded RNA (dsRNA), small-interfering RNA (siRNA) or ribozyme.

In such embodiment, the particular cleaving component(s) may remove the cap structure from the intact exogenous RNA molecule, for reducing the efficiency of translation of the exogenous protein of interest in the intact exogenous RNA molecule. For example, see FIG. 9C.

In another embodiment, the inhibitory sequence that is located upstream from the cleavage site may further comprise one or more initiation codon(s), such that each of the initiation codon(s) and the sequence encoding the exogenous protein of interest are not in the same reading frame and such that each of these initiation codon(s) is located within a Kozak consensus sequence.

### 3. Structure of the Exogenous RNA Molecule Having its Inhibitory Sequence Located Downstream from the Cleavage Site

3.1. Structure of the Inhibitory Sequence that is Located Downstream from the Cleavage Site

According to some embodiments, the inhibitory sequence in the exogenous RNA molecule may be located upstream or downstream from the cleavage site. This section describes embodiments wherein the inhibitory sequence is located downstream from the cleavage site in the exogenous RNA molecule. For example, see FIG. 3.

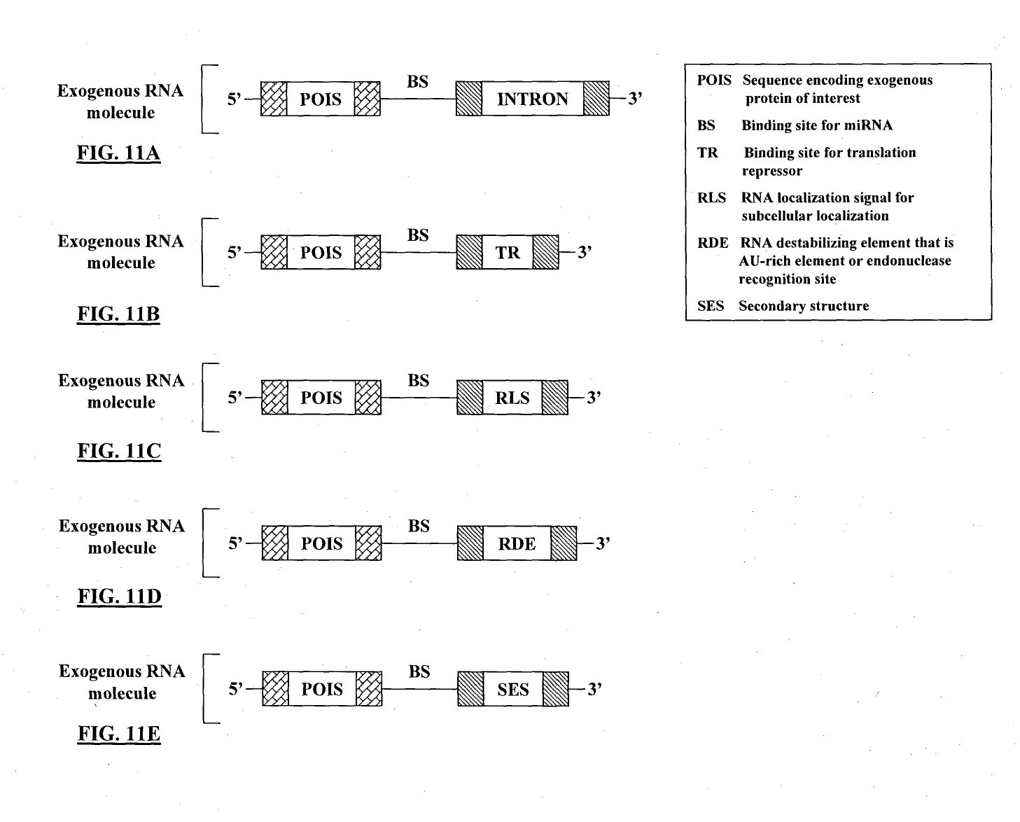

In some embodiments, the inhibitory sequence that is located downstream from the cleavage site may comprise, for example, an intron. The exogenous RNA molecule may thus be target for nonsense-mediated decay (NMD) that degrades the exogenous RNA molecule [29]. For example, see FIG. 11A.

In one embodiment, the inhibitory sequence that is located downstream from the cleavage site may comprise a sequence that is capable of binding to a translation repressor protein, such that the translation repressor protein is an endogenous translation repressor protein or is encoded from the composition and such that the translation repressor protein may, directly or indirectly, reduce the efficiency of translation of the exogenous protein of interest within the exogenous RNA molecule [24]. The sequence that is capable of binding to a translation repressor protein may be, for example, but is not limited to a binding sequence of smaug repressor protein (5′-UGGAGCAGAGGCUCUGGCAGCUUUUGCAGCG-3′-SEQ ID NO. 28) [25]. For example, see FIG. 11B.

In another embodiment, the inhibitory sequence that is located downstream from the cleavage site may comprise an RNA localization signal for subcellular localization (including cotranslational import) or an endogenous miRNA binding site, such that the subcellular localization of the exogenous RNA molecule may inhibit the translation of the exogenous protein of interest and may decrease the exogenous RNA molecule half-life. The RNA localization signal may comprise, for example, but is not limited to an RNA localization signal for: myelinating periphery, myelin compartment, leading edge of the lamella, mitochondria or Perinuclear cytoplasm [22]. The RNA localization signal may be, for example, but is not limited to RNA localization signal for myelinating periphery 5′-GCCAAGGAGCCAGAGAGCAUG-3′ (SEQ ID NO. 29) or 5′-GCCAAGGAGCC-3′ (SEQ ID NO. 30) [27]. For example, see FIG. 11C.

In another embodiment, the inhibitory sequence that is located downstream from the cleavage site may comprise an RNA destabilizing element that may stimulate degradation of the exogenous RNA molecule, such that the RNA destabilizing element is an AU-rich element (ARE) or an endonuclease recognition site. The AU-rich element may be, for example, but is not limited to AU-rich elements that are at least about 35 nucleotides long. The AU-rich element may be, for example, 5′-AUUUA-3′ (SEQ ID NO. 31), 5′-UUAUUUA(U/A)(U/A)-3′ (SEQ ID NO. 32) or 5′-AUUU-3′ (SEQ ID NO. 33) [26]. For example, see FIG. 11D.

In another embodiment, the inhibitory sequence that is located downstream from the cleavage site may comprise a sequence that is capable of forming a secondary structure that may reduce the efficiency of translation of the upstream exogenous protein of interest. For example, see FIG. 11E.

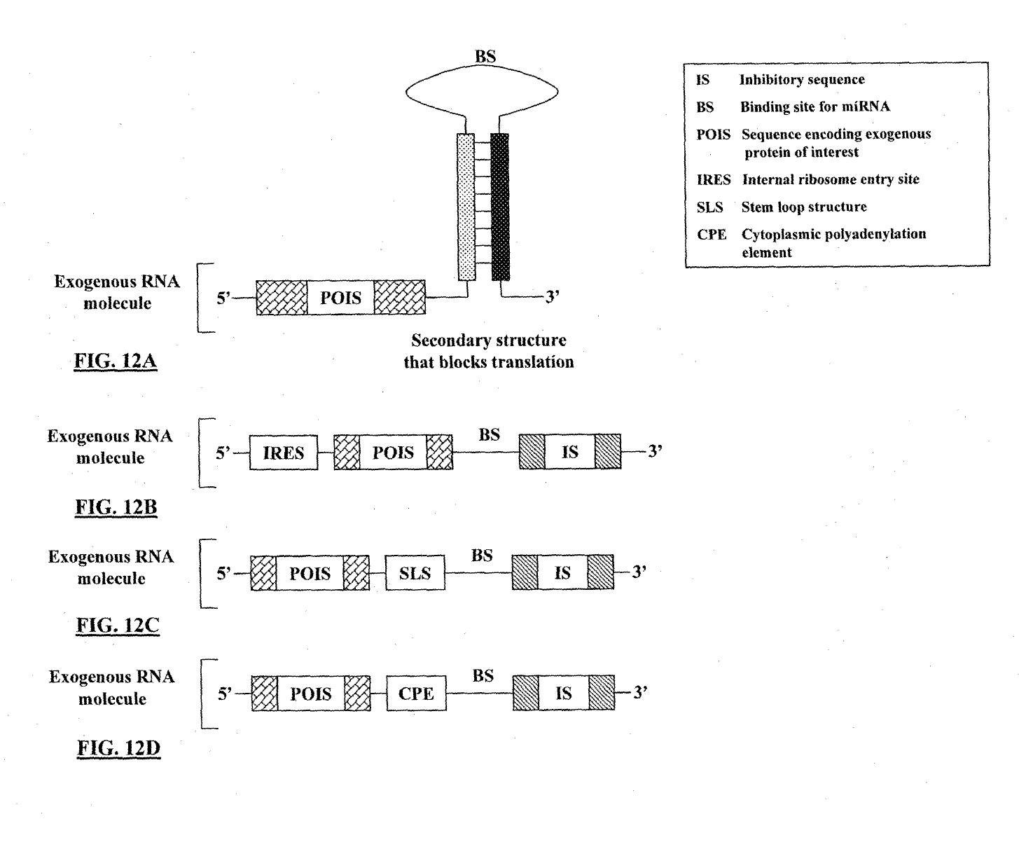

In another embodiment, inhibitory sequence that is located downstream from the cleavage site may comprise a sequence immediately downstream from the cleavage site that is capable of binding to the nucleotide sequence that is located immediately upstream from the cleavage site, for the formation of a secondary structure. The secondary structure, directly or indirectly, may reduce the efficiency of translation of the upstream exogenous protein of interest. In some embodiments, the folding free energy of the secondary structure may be is lower than −30 kcal/mol (for example, −50 kcal/mol, −80 kcal/mol) and thus this secondary structure is sufficient to block scanning ribosomes from reaching the stop codon of the exogenous protein of interest. In another embodiment, the cleavage site is located within a single stranded region or within the loop region in the secondary structure, such that the single stranded region or the loop region may be, for example, but is not limited to, a region that is at least about 15 nucleotides long. For example, see FIG. 12A.

3.2. Additional Structures that May Increase the Efficiency of Translation of the Exogenous RNA Molecule that is Cleaved at the Cleavage Site at the 3′ End

This section describes embodiments of additional structures such that these additional structures may increase the efficiency of translation of the cleaved exogenous RNA molecule, wherein the cleaved exogenous RNA molecule is cleaved at the cleavage site at the 3′ end.

In some embodiments, the exogenous RNA molecule may comprise a sequence that has a unique internal ribosome entry site (IRES) sequence immediately upstream from the sequence encoding the exogenous protein of interest, such that the unique IRES sequence may increase the efficiency of translation of the exogenous protein of interest in the cleaved exogenous RNA molecule. For example, see FIG. 12B.

In another embodiment of the invention the exogenous RNA molecule may comprise a unique nucleotide sequence immediately downstream from the sequence encoding the exogenous protein of interest, such that the unique nucleotide sequence comprises a unique stem loop structure and such that the unique stem loop structure, directly or indirectly, may increase the efficiency of translation of the exogenous protein of interest and the exogenous RNA molecule half-life of the cleaved exogenous RNA molecule. The unique stem loop structure may be, for example, but is not limited to the conserved stem loop structure of the human histone gene 3′-UTR or a functional derivative thereof. The conserved stem loop structure of the human histone gene 3-UTR is 5′-GGCUCUUUUCAGAGCC-3′ (SEQ ID NO. 34). For example, see FIG. 12C.

In one embodiment of the invention, the exogenous RNA molecule that is described in section 3.1 or 1 may comprise a unique nucleotide sequence immediately downstream from the sequence encoding the exogenous protein of interest, such that the unique nucleotide sequence includes a cytoplasmic polyadenylation element that, directly or indirectly, may increase the efficiency of translation of the exogenous protein of interest and the exogenous RNA molecule half-life in the cleaved exogenous RNA molecule. The cytoplasmic polyadenylation element may be, for example, but is not limited to 5′-UUUUAU-3′ (SEQ ID NO. 35), 5′-UUUUUAU-3′ (SEQ ID NO. 36), 5′-UUUUAAU-3′ (SEQ ID NO. 37), 5′-UUUUUUAUU-3′ (SEQ ID NO. 38), 5′-UUUUAUU-3′ (SEQ ID NO. 39) or 5′-UUUUUAUAAAG-3′ (SEQ ID NO. 40) [23]. The composition of the invention may also comprise a polynucleotide sequence that encodes a human cytoplasmic polyadenylation element binding protein (hCPEB), or a homologue thereof for expressing hCPEB in any cell. For example, see FIG. 12D.

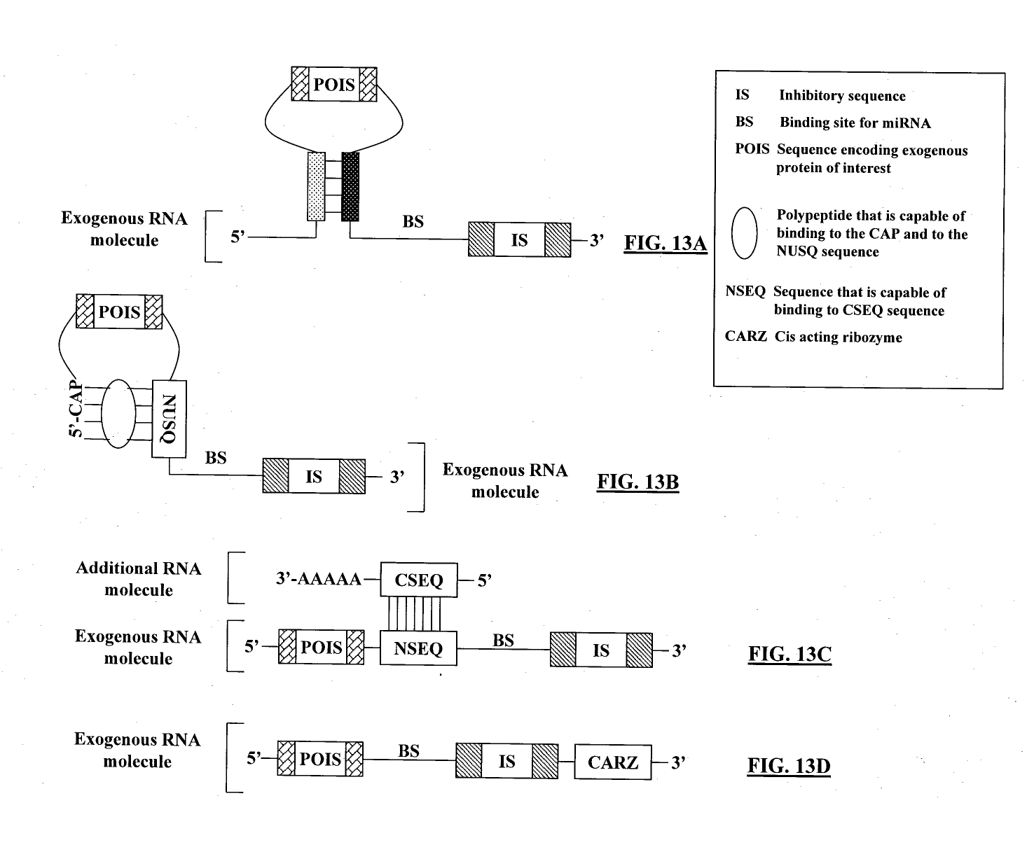

In some embodiments, the exogenous RNA molecule may comprise a unique nucleotide sequence that is located upstream from the cleavage site and downstream from the sequence encoding the exogenous protein of interest, such that the unique nucleotide sequence is capable of binding to a sequence that is located upstream from the sequence encoding the exogenous protein of interest. In this embodiment, the cleaved exogenous RNA molecule may create a circular structure that may increase the efficiency of translation of the exogenous protein of interest in the cleaved exogenous RNA molecule. For example, see FIG. 13A.

In another embodiment, the exogenous RNA molecule may comprise a unique nucleotide sequence that is located upstream from the cleavage site and downstream from the sequence encoding the exogenous protein of interest. The unique nucleotide sequence may be capable of binding to a unique polypeptide that is, directly or indirectly, capable of binding to the CAP structure in the cleaved exogenous RNA molecule. The unique polypeptide may also be encoded from the composition of the invention. In this embodiment, the unique polypeptide and the cleaved exogenous RNA molecule may create a circular structure that may increase the efficiency of translation of the exogenous protein of interest in the cleaved exogenous RNA molecule. For example, see FIG. 13B.

In further embodiments, the composition of the invention may comprise an additional polynucleotide sequence, which may encode for an additional RNA molecule that has at the 3′ end a nucleotide sequence that is capable of binding to a sequence that is located upstream from the cleavage site and downstream from the sequence encoding the exogenous protein of interest. The expression of the additional polynucleotide sequence may be driven by a polymerase II based promoter. In this embodiment the additional RNA molecule may be capable of binding to the cleaved exogenous RNA molecule and provide it with a poly-A tail which may increase the efficiency of translation of the exogenous protein of interest from the cleaved exogenous RNA molecule. For example, see FIG. 13C.

3.3. Additional Structures that May Reduce the Efficiency of Translation of the Intact Exogenous RNA Molecule

This section describes embodiments for additional structures that may reduce the efficiency of translation of the intact exogenous RNA molecule, before it is cleaved.

In some embodiments, the composition may further comprise a particular cleaving component(s) that is capable of effecting the cleavage, directly or indirectly, of the exogenous RNA molecule at a position that is located downstream from the inhibitory sequence, wherein the inhibitory sequence is located downstream from the cleavage site. The particular cleaving component(s) may comprise, for example:
(a) a particular nucleic acid sequence that is located within the exogenous RNA molecule, such that the particular nucleic acid sequence may be selected from, but is not limited to: endonuclease recognition site, endogenous miRNA binding site, cis acting ribozyme or miRNA sequence; or
(b) a particular inhibitory RNA that is encoded from the composition of the invention, such that the particular inhibitory RNA may be selected from, but is not limited to: microRNA (miRNA), lariat-form RNA, short-hairpin RNA (shRNA), siRNA expression domain, antisense RNA, double-stranded RNA (dsRNA), small-interfering RNA (siRNA) or ribozyme.

In this embodiment, the particular cleaving component(s) may remove the poly-A tail from the intact exogenous RNA molecule for reducing the efficiency of translation of the exogenous protein of interest in the intact exogenous RNA molecule. For example, see FIG. 13D.

### 4. Clarifications and Additional Embodiments

The term “sufficient complementarity” may include, but is not limited to being capable of binding or at least partially complementary. In some embodiments, the term sufficient complementarity is in the range of about 30-100%. For example, in some embodiments, the term sufficient complementarity is at least 30% complementarity. For example, in some embodiments, the term sufficient complementarity is at least 50% complementarity. For example, in some embodiments, the term sufficient complementarity is at least 70% complementarity. For example, in some embodiments, the term sufficient complementarity is at least 90% complementarity. For example, in some embodiments, the term sufficient complementarity is about 100% complementarity.

According to some embodiments, the cell into which the composition of the invention may be inserted/introduced into may be, for example, but is not limited to: human cell, animal cell, cultured cell, plant cell, primary cell, a cell that is present in an organism.

In some embodiments, the specific endogenous miRNA that cleaves the exogenous RNA molecule, may be, for example, but is not limited to: microRNA that is unique to a specific cell type, miRNA that is unique to neoplastic cells, viral microRNA, or the like. The viruses that encode the viral miRNA may be selected from, for example, but are not limited to: double-stranded DNA virus, a single-stranded DNA virus, a double-stranded RNA virus, a double-stranded RNA virus, a single-stranded (plus-strand) virus, a single-stranded (minus-strand) virus or a retrovirus.

In some exemplary embodiments, the specific endogenous miRNA that cleaves the exogenous RNA molecule may be selected from, for example, but is not limited to: miR-17-92, miR-221, miR-222, miR-146, miR-221, miR-21, miR-155, mir 675, miR-10b, hsv1-miR-H1, hsv1-miR-H2, hsv1-miR-H3, hsv1-miR-H4, hsv1-miR-H5, hsv1-miR-H6, hsv2-miR-I, hcmv-miR-UL22A, hcmv-miR-UL36, hcmv-miR-UL70, hcmv-miR-UL112, hcmv-miR-UL148D, hcmv-miR-US4, hcmv-miR-US5-1, hcmv-miR-US5-2, hcmv-miR-US25-1, hcmv-miR-US25-2, hcmv-miR-US33, kshv-miR-K12-1, kshv-miR-K12-2, kshv-miR-K12-3, kshv-miR-K12-4, kshv-miR-K12-5, kshv-miR-K12-6, kshv-miR-K12-7, kshv-miR-K12-8, kshv-miR-K12-9, kshv-miR-K12-10a, kshv-miR-K12-10b, kshv-miR-K12-11, kshv-miR-K12-12, ebv-miR-BART1, ebv-miR-BART2, ebv-miR-BART3, ebv-miR-BART4, ebv-miR-BART5, ebv-miR-BART6, ebv-miR-BART7, ebv-miR-BART8, ebv-miR-BART9, ebv-miR-BART10, ebv-miR-BART11, ebv-miR-BART12, ebv-miR-BART13, ebv-miR-BART14, ebv-miR-BART15, ebv-miR-BART16, ebv-miR-BART17, ebv-miR-BART18, ebv-miR-BART19, ebv-miR-BART20, ebv-miR-GHRF1-1, ebv-miR-BHRF1-2, ebv-miR-BHRF1-3, bkv-miR-B1, jcv-miR-J1, hiv1-miR-H1, hiv1-miR-N367, hiv1-miR-TAR, sv40-miR-S1, MCPyV-miR-M1, hsv1-miR-LAT, hsv1-miR-LAT-ICP34.5, hsv2-miR-III, hcmv-miR-UL23, hcmv-miR-UL36-1, hcmv-miR-UL54-1, hcmv-miR-UL70-1, hcmv-miR-UL22A-1, hcmv-miR-UL112-1, hcmv-miR-UL148D-1, hcmv-miR-US4-1, hcmv-miR-US24, hcmv-miR-US33-1, hcmv-RNAβ2.7, ebv-miR-BART1-1, ebv-miR-BART1-2, ebv-miR-BART1-3, ebv-miR-BHFR1, ebv-miR-BHFR2, ebv-miR-BHFR3, hiv1-miR-TAR-5p, hiv1-miR-TAR-p, hiv1-HAAmiRNA, hiv1-VmiRNA1, hiv1-VmiRNA2, hiv1-VmiRNA3, hiv1-VmiRNA4, hiv1-VmiRNA5, hiv2-miR-TAR2-5p, hiv2-miR-TAR2-3p, mdv1-miR-M1, mdv1-miR-M2, mdv1-miR-M3, mdv1-miR-M4, mdv1-miR-M5, mdv1-miR-M6, mdv1-miR-M7, mdv1-miR-M8, mdv1-miR-M9, mdv1-miR-M10, mdv1-miR-M11, mdv1-miR-M12, mdv1-miR-M13, mdv2-miR-M14, mdv2-miR-M15, mdv2-miR-M 16, mdv2-miR-M 17, mdv2-miR-M 18, mdv2-miR-M 19, mdv2-miR-M20, mdv2-miR-M21, mdv2-miR-M22, mdv2-miR-M23, mdv2-miR-M24, mdv2-miR-M25, mdv2-miR-M26, mdv2-miR-M27, mdv2-miR-M28, mdv2-miR-M29, mdv2-miR-M30, mcmv-miR-M23-1, mcmv-miR-M23-2, mcmv-miR-M44-1, mcmv-miR-M55-1, mcmv-miR-M87-1, mcmv-miR-M95-1, mcmv-miR-m01-1, mcmv-miR-m01-2, mcmv-miR-m01-3, mcmv-miR-m01-4, mcmv-miR-m21-1, mcmv-miR-m22-1, mcmv-miR-m59-1, mcmv-miR-m59-2, mcmv-miR-m88-1, mcmv-miR-m107-1, mcmv-miR-m108-1, mcmv-miR-m108-2, rlcv-miR-rL1-1, rlcv-miR-rL1-2, rlcv-miR-rL1-3, rlcv-miR-rL1-4, rlcv-miR-rL1-5, rlcv-miR-rL1-6, rlcv-miR-rL1-7, rlcv-miR-rL1-8, rlcv-miR-rL1-9, rlcv-miR-rL1-10, rlcv-miR-rL1-11, rlcv-miR-rL1-12, rlcv-miR-rL1-13, rlcv-miR-rL1-14, rlcv-miR-rL1-15, rlcv-miR-rL1-16, rrv-miR-rR1-1, rrv-miR-rR1-2, rrv-miR-rR1-3, rrv-miR-rR1-4, rrv-miR-rR1-5, rrv-miR-rR1-6, rrv-miR-rR1-7, mghv-miR-M 1-1, mghv-miR-M 1-2, mghv-miR-M 1-3, mghv-miR-M 1-4, mghv-miR-M 1-5, mghv-miR-M 1-6, mghv-miR-M 1-7, mghv-miR-M 1-8, mghv-miR-M1-9 or sv40-miR-S1 [34, 35]. The nomenclature and sequences of the various miRNA molecules are as defined at the database http://www.mirbase.org/.

According to some embodiments, the exogenous protein of interest that is encoded from the exogenous RNA molecule may be any type of protein. For example, the exogenous protein of interest may by selected from, but not limited to: alpha toxin, saporin, maize RIP, barley RIP, wheat RIP, corn RIP, rye RIP, flax RIP, Shiga toxin, Shiga-like RIP, momordin, pokeweed antiviral protein, gelonin, Pseudomonas exotoxin, Pseudomonas exotoxin A or modified forms thereof, Ricin A chain, Abrin A chain, Diphtheria toxin fragment A or modified forms thereof, a fluorescent protein, an enzyme (such as, for example, Luciferase), a structural protein, or the like.

In some embodiments the exogenous protein of interest may be a toxin that can also effect neighboring cells. For example, the toxin may be selected from, but not limited to, the complete form of: Ricin, Abrin, Diphtheria toxin or modified forms thereof. In some embodiments, the exogenous protein of interest may be, for example, an enzyme, the product of which may kill also the neighboring cells. Such an enzyme may be, for example, but is not limited to: HSV1 thymidine kinase. In some embodiments, the composition of the invention may further comprise the prodrug-ganciclovir, which is a substrate for the HSV1 thymidine kinase. In some exemplary embodiments, the enzyme may be Escherichia coli cytosine deaminase, and the composition may further comprise the prodrug-5-fluorocytosine (5-FC).

In some embodiments, the sequence encoding the exogenous protein of interest may comprise, in addition to the coding region of the exogenous protein of interest, one or more introns that may increase the expression of the protein of interest. In some embodiments, the intron may be an intron which is part of the natural gene encoding the protein of interest. In some embodiments, the intron may be an intron of an unrelated gene. In some embodiments, the exogenous RNA molecule may be encoded from any expression vector. For example, the exogenous RNA molecule may be encoded from a viral vector and the exogenous protein of interest may be is a product of gene that is necessary for the viral vector reproduction, such that the viral vector reproduces in response to the presence of the specific endogenous miRNA in a cell and kills the cell during the process of reproduction. The viral vector may also comprise, for example, a gene that is capable of stopping the viral vector reproduction when a specific molecule is present in the cell (for example, TetR-VP16/Doxycycline). Such that when the viral vector is presumed to get enough mutations for reproduction in cells that do not include the specific endogenous miRNA, the specific molecule can be administered for stopping all the viral vectors reproduction in the body and then after the degradation of most of the viral vectors in the body, new viral vectors can be administered again. The viral vector may also include, for example, a gene that is capable of killing the cell when a specific prodrug is present (e.g. thymidine kinase/ganciclovir), such that when the viral vector is presumed to get enough mutations for reproduction in cells that do not include the specific endogenous miRNA, the specific prodrug can be administered for killing all the viral vectors in the body and then new viral vectors can be administered again.

In some exemplary embodiments, the exogenous RNA molecule may be encoded from a viral vector that is capable of being reproduced in a manner that kills the cell during the process of reproduction. In this embodiment, the specific endogenous miRNA is not present in the target cells (for example, cancer cells) of a patient, but rather the specific endogenous miRNA is present in most of the normal or nonmetastatic tumourigenic cells of the patient. In this example, the exogenous protein of interest is a toxin, such as, for example, Ricin A chain, Abrin A chain, Diphtheria toxin fragment A or modified forms thereof. When the viral vector enters a normal or nonmetastatic tumourigenic cell it kills the cell and when the viral vector enters a target cell (cancer cell), it kills the cancer cell during the process of the viral vector reproduction, thus the major concentration of the viral vector is present in the tumor region. This viral vector may also include, gene that is capable of stopping the viral vector reproducing when a specific molecule is present in the cell (for example, TetR-VP16/Doxycycline). Such that when the viral vector is presumed to get enough mutations for reproduction in cells that comprise the specific endogenous miRNA the specific molecule can be administered for stopping all the viral vectors reproduction in the body and then after the degradation of most of the viral vectors in the body cells new viral vectors can be administered again. This viral vector may also include a gene that is capable of killing the cell when a specific prodrug is present (for example, thymidine kinase/ganciclovir), such that when the viral vector is presumed to get enough mutations for reproduction in cells that comprise the specific endogenous miRNA the specific prodrug can be administered for killing all the viral vectors in the body and then new viral vectors can be administered again.

According to some embodiments, the inhibitory sequence may be a sequence or a part of a sequence that, upon detaching from the sequence encoding the exogenous protein of interest, the exogenous protein of interest is capable of being expressed. When the inhibitory sequence is not detached from the sequence encoding the exogenous protein of interest, it is capable of inhibiting the expression of the exogenous protein of interest, when it is within its specific context in the exogenous RNA molecule. The inhibitory sequence may also include only a part of any of the inhibitory sequences described above, within its specific context. For example, instead of an inhibitory sequence that is an out of reading frame 5′-AUG-3′ the inhibitory sequence may be only the A or the 5′-AU-3′ part in the context of -UG-3′ or -G-3′ respectively (that is, the exogenous RNA molecule comprises an out of reading frame 5′-AUG-3′ at the 5′ end, however the sequence that will be detached is only the 5′-AU-3′ part).

In another embodiment of the invention, the composition of the invention may further comprise a polynucleotide sequence encoding a special functional RNA that is capable of inhibiting the expression, directly or indirectly, of an endogenous exonuclease. The special functional RNA may be, for example, but is not limited to: microRNA (miRNA), lariat-form RNA, short-hairpin RNA (shRNA), siRNA expression domain, antisense RNA, double-stranded RNA (dsRNA), small-interfering RNA (siRNA) or ribozyme.

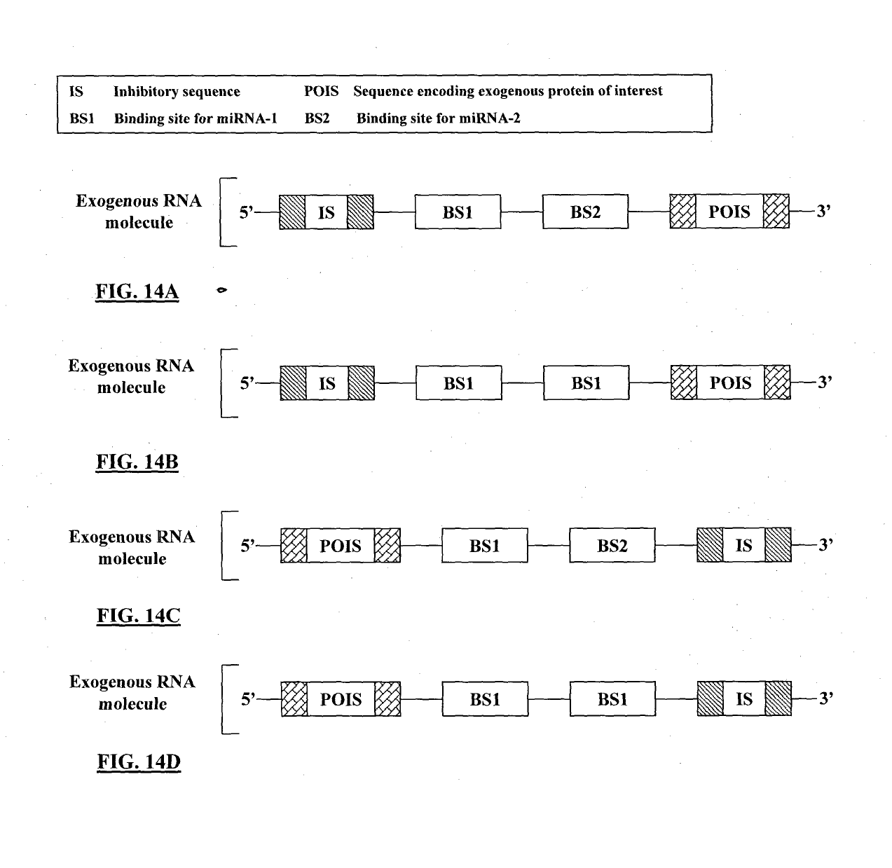

In another embodiment of the invention, the binding site described above may be a plurality of binding sites for the same or different miRNAs, such that wherein said “upstream from the cleavage site” it also encompasses “upstream from all the cleavage sites”. Likewise, wherein said “downstream from the cleavage site” also encompasses “downstream from all the cleavage sites”. In some embodiments, when the plurality of binding sites are for different endogenous miRNAs, the exogenous protein of interest may be expressed even if only one of the miRNAs is present within the cell. For example, see FIG. 14A, 14B, 14C, 14D.

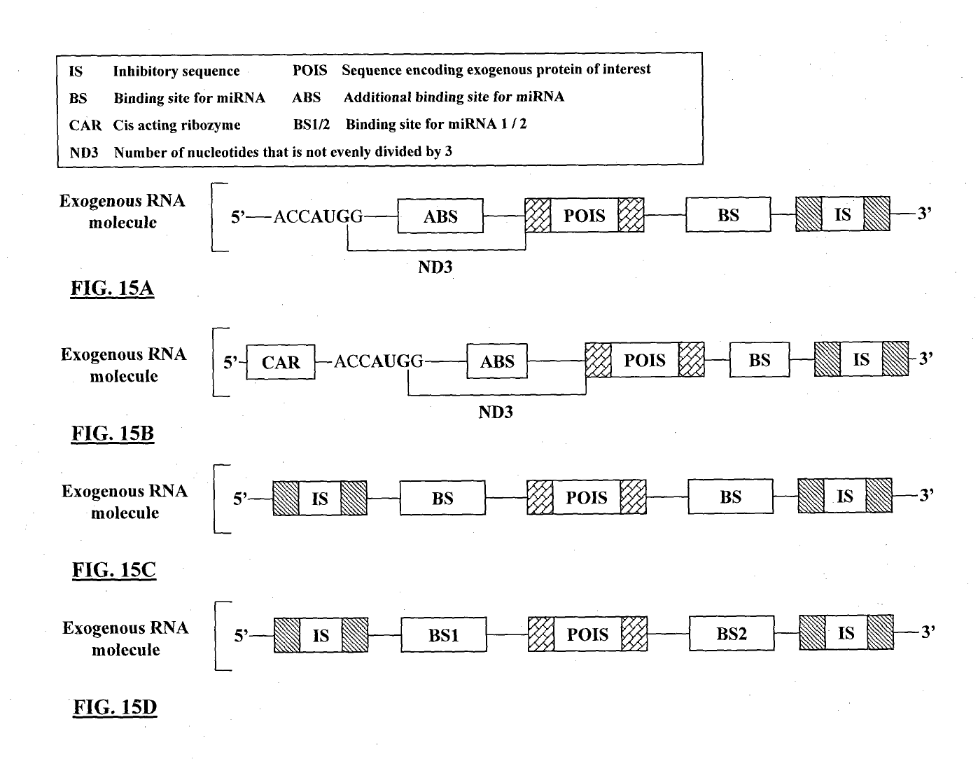

In some embodiments of the invention, the exogenous RNA molecule may further comprise one or more additional binding site(s) for the specific endogenous miRNA, such that each of the additional binding site(s) is of sufficient complementarity for the specific endogenous miRNA to direct cleavage of the exogenous RNA molecule at unique cleavage site(s) via RNA interference. Each of the unique cleavage site(s) may be located within each of the additional binding site(s) and each of the unique cleavage site(s) may be located upstream from the sequence encoding the exogenous protein of interest. The exogenous RNA molecule may further comprise one or more initiation codon(s) upstream from all the unique cleavage site(s), such that each of the initiation codon(s) and the sequence encoding the exogenous protein of interest are not in the same reading frame. The initiation codon(s) may, for example, be consisting essentially of 5′-AUG-3′, such that at least one of the initiation codon(s) is located within a Kozak consensus sequence or any other translation initiation element. The initiation codon may be, for example, a TISU element [38]. According to some embodiments, following introduction of the composition into a cell comprising the specific endogenous miRNA, the exogenous RNA molecule may transcribed and cleaved by the specific endogenous miRNA at the cleavage site and at each of the unique cleavage site(s) such that the sequence encoding the exogenous protein of interest is detached from the inhibitory sequence and from each of the initiation codon(s) and the exogenous protein of interest is capable of being expressed. For example, see FIG. 15A.

In some embodiments, the composition of the invention may further comprise a cleaving component(s) that is capable of effecting the cleavage, directly or indirectly, of the exogenous RNA molecule at a position that is located upstream from each of the initiation codon(s), such that the cleaving component(s) is, for example:
(a) a nucleic acid sequence that is located within the exogenous RNA molecule, such that the nucleic acid sequence is: endonuclease recognition site, endogenous miRNA binding site, cis acting ribozyme or miRNA sequence; or
(b) an inhibitory RNA that is encoded from the composition, such that the inhibitory RNA is: microRNA (miRNA), lariat-form RNA, short-hairpin RNA (shRNA), siRNA expression domain, antisense RNA, double-stranded RNA (dsRNA), small-interfering RNA (siRNA) or ribozyme. For example, see FIG. 15B.

According to some embodiments, the composition of the invention may comprise one or more polynucleotide molecules, such as, for example, DNA molecules, RNA molecules, or both. In one embodiment, the composition may comprise a DNA molecule for expressing an exogenous protein of interest in a cell, only in the presence of a specific endogenous miRNA in the cell, wherein the specific endogenous miRNA may be, for example, a cellular miRNA, a viral miRNA, or the like. The DNA molecule may comprise polynucleotide sequence that encodes for an exogenous RNA molecule, the exogenous RNA molecule is an RNA molecule that comprises: a sequence encoding the exogenous protein of interest, a binding site(s) for the specific endogenous miRNA, upstream from the sequence encoding the exogenous protein of interest, additional binding site(s) for the specific endogenous miRNA, downstream from the sequence encoding the exogenous protein of interest and at least two inhibitory sequences—one at the 5′ end of the exogenous RNA molecule and the other at the 3′ end of the exogenous RNA molecule, such that each of the inhibitory sequences is capable of inhibiting the expression of the exogenous protein of interest. Thus, only when the specific endogenous miRNA is present in a cell, the two inhibitory sequences may be detached from the sequence encoding the exogenous protein of interest and the exogenous protein of interest is capable of being expressed in the cell. The inhibitory sequences may be any of the sequences described above. For example, see FIG. 15C.

According to further embodiments, the composition may comprise a DNA molecule for expressing an exogenous protein of interest in a cell only in the presence of two specific endogenous miRNAs in a cell. The DNA molecule may comprise a polynucleotide sequence that encodes for an exogenous RNA molecule, the exogenous RNA molecule is an RNA molecule that comprises: a sequence encoding the exogenous protein of interest, a binding site(s) for the first specific endogenous miRNA upstream from the sequence encoding the exogenous protein of interest, another binding site(s) for the second specific endogenous miRNA downstream from the sequence encoding the exogenous protein of interest and at least two inhibitory sequences, one at the 5′ end of the exogenous RNA molecule and other at the 3′ end of the exogenous RNA molecule. Each of the inhibitory sequences may be capable of inhibiting the expression of the exogenous protein of interest, such that when the two specific endogenous miRNAs are present in the cell, the two inhibitory sequences may be detached from the sequence encoding the exogenous protein of interest, and the exogenous protein of interest may be capable of being expressed in the cell. The inhibitory sequences may be any of the sequences described above. For example, see FIG. 15D.

According to additional embodiments, when there is a need to express the exogenous protein of interest only when plurality of different miRNAs are present simultaneously in a cell, the composition of the invention may comprise or encode for a plurality of exogenous RNA molecules, wherein the structure of each of the exogenous RNA molecules may be as described above. The exogenous RNA molecules may be similar or different. Each of these exogenous RNA molecules may comprise different miRNA binding site and different sequences encoding different proteins of interest, such that all the different proteins of interest may together create a new function in the cell. For example, when the plurality of different miRNAs includes three different miRNAs, the three different proteins of interest expressed from the three different exogenous RNA molecules, may be selected from: protective antigen (PA), edema factor (EF) and the lethal factor (LF), such that when the three different miRNAs are present simultaneously in the cell, the 3 proteins: protective antigen (PA), edema factor (EF) and the lethal factor (LF) are expressed and create together the Anthrax toxin that may induce cell death.

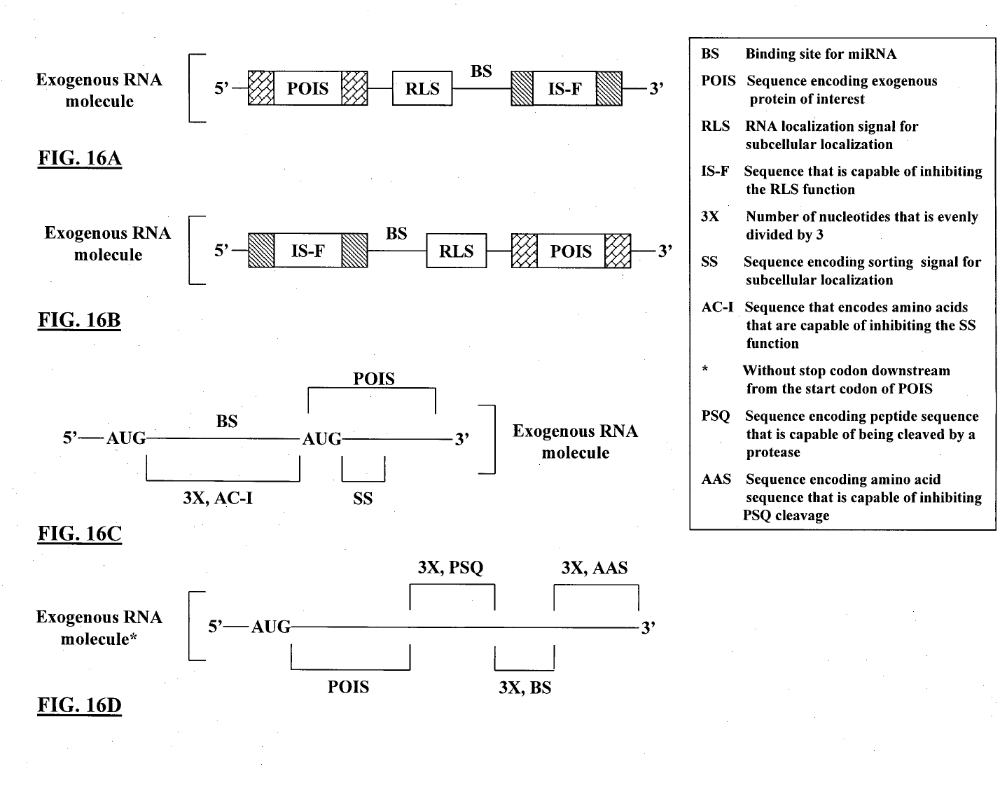

In another embodiment, the exogenous RNA molecule may further have an RNA localization signal for subcellular localization (including cotranslational import) between the cleavage site and the sequence encoding the exogenous protein of interest, such that the inhibitory sequence is capable of inhibiting the function of the RNA localization signal for subcellular localization and such that the subcellular localization of the exogenous RNA molecule is necessary for the proper expression of the exogenous protein of interest. For example, see FIG. 16A, 16B.

In further embodiment, the inhibitory sequence may comprise an initiation codon upstream from the cleavage site, wherein the initiation codon is consisting essentially of 5′-AUG-3′. The inhibitory sequence may further comprise a nucleotide sequence encoding an amino acid sequence immediately downstream from the initiation codon, such that the nucleotide sequence and the sequence encoding the exogenous protein of interest are in the same reading frame. The amino acid sequence may be capable of inhibiting the function of the sorting signal for subcellular localization of the exogenous protein of interest, wherein the subcellular localization of the exogenous protein of interest is necessary for its proper expression. For example, see FIG. 16C.

In another embodiment of the invention, the exogenous RNA molecule does not include a stop codon downstream from the start codon of the sequence encoding the exogenous protein of interest. The inhibitory sequence may be located downstream from the sequence encoding the exogenous protein of interest, such that the inhibitory sequence and the sequence encoding the exogenous protein of interest are in the same reading frame, and the inhibitory sequence encodes an amino acid sequence that is selected from the group consisting of:
(a) an amino acid sequence that is capable of inhibiting the function of the exogenous protein of interest;
(b) an amino acid sequence that is a sorting signal for subcellular localization;
(c) an amino acid sequence that is a protein degradation signal;
(d) an amino acid sequence that is capable of inhibiting the function of the sorting signal for subcellular localization of the exogenous protein of interest; and
(e) an amino acid sequence that is capable of inhibiting the cleavage of a peptide sequence that is encoded by a nucleotide sequence that is located between the cleavage site and the start codon of the sequence encoding the exogenous protein of interest, such that the nucleotide sequence and the sequence encoding the exogenous protein of interest are in the same reading frame and such that the peptide sequence is capable of being cleaved by a protease in a mammalian cell. (It has been reported that in the human cell during translation of truncated mRNA without stop codon(s), the ribosome stalls at the terminal codon and the cognate tRNA molecule remains bound to the polypeptide chain and to the ribosome, however, it is possible for a peptidyl-tRNA species, in the midst of translation, to be processed by the endoplasmic reticulum signal peptidase [32]) For example, see FIG. 16D.

### 5. Synthesis of the Composition of the Invention

According to some embodiments, and as detailed above, the composition may comprise one or more polynucleotide molecules that include or encode for the exogenous RNA molecule. The polynucleotide molecules may be one or more DNA molecules, one or more RNA molecules, or combinations thereof. In some exemplary embodiments, the composition may comprise one or more DNA molecule that encode for the exogenous RNA molecule. The DNA molecule that encodes the exogenous RNA molecule may be recombinantly engineered into a variety of host vector systems/constructs that may also provide for replication of the DNA in large scale and contain the necessary elements for directing the transcription of the exogenous RNA molecule. The introduction of such vectors to target cells results in the transcription of sufficient amounts of the exogenous RNA molecule within the cell. For example, a vector can be introduced in vivo such that it is taken up by a cell and directs the transcription of the exogenous RNA molecule. Such a vector can remain episomal or become chromosomally integrated, as long as it can be transcribed to produce the desired exogenous RNA molecule. Such vectors can be constructed by recombinant DNA technology methods well known in the art or can be prepared by any method known in the art for the synthesis of DNA molecules.

According to some embodiments, the recombinant DNA constructs that encode for the exogenous RNA molecule can include, for example plasmid, cosmid, viral vector, or any other vector known in the art, used for replication and expression in the desired target cells (such as, for example, mammalian cells (for example, human cells, murine cells), avian cells, plant cells, and the like). Expression of the exogenous RNA molecule can be regulated by any promoter known in the art to act in the desired target cells. Such promoters can be inducible or constitutive. Such promoters include, for example, but are not limited to: the SV40 early promoter region, the promoter contained in the 3′ long terminal repeat of Rous sarcoma virus, the herpes thymidine kinase promoter, the regulatory sequences of the metallothionein gene, the viral CMV promoter, the human chorionic gonadotropin-beta promoter, etc. In some embodiments, the promoter may be an RNA Polymerase I promoter (i.e., a promoter that is recognized by RNA Pol. I), such as, for example, the promoter of ribosomal DNA (rDNA) gene. In such embodiments, the termination signal of the exogenous RNA of interest molecule may be an RNA Pol. I termination signal or a RNA polymerase II termination signal (such as, for example, a polyA signal). Any type of plasmid, cosmid, YAC or viral vector can be used to prepare the recombinant DNA constructs which can be introduced directly into a target cell/cell population or to a the tissue site. Alternatively, viral vectors can be used which selectively infect the desired target cell.

According to some embodiments, for the formation of a transgenic organism that is resistant to viral infection or cancer, it is desirable that the vector that encodes the exogenous RNA molecule will have a selectable marker. A number of selection systems can be used, including but not limited to selection for expression of the herpes simplex virus thymidine kinase, hypoxanthine-guanine phosphoribosyltransterase and adenine phosphoribosyl tranferase protein in tk-, hgprt- or aprt-deficient cells, respectively. Also, anti-metabolic resistance can be used as the basis of selection for dihydrofolate tranferase (dhfr), which confers resistance to methotrexate; xanthine-guanine phosphoribosyl transferase (gpt), which confers resistance to mycophenolic acid; neomycin (neo), which confers resistance to aminoglycoside G-418; and hygromycin B phosphotransferase (hygro) which confers resistance to hygromycin.

According to some embodiments, vectors for use in the practice of the invention may be any expression vector. In some exemplary embodiments, the exogenous RNA molecule is encoded by a viral expression vector. The viral expression vector may be selected from, but is not limited to: Herpesviridae, Poxyiridae, Adenoviridae, Papillomaviridae, Parvoviridae, Hepadnoviridae, Retroviridae, Reoviridae, Filoviridae, Paramyxoviridae, Pneumoviridae, Rhabdoviridae, Orthomyxoviridae, Bunyaviridae, Hantaviridae, Picornaviridae, Caliciviridae, Togaviridae, Flaviviridae, Arenaviridae, Coronaviridae, or Hepaciviridae. The viral expression vector may also include, but is not limited to an adenoviral vector that its cellular tropism has been modified by the replacement of the adenovirus terminal knob domain of the fiber protein (HI loop), which is exposed at the fiber surface.

In some embodiments, the composition of the invention may comprise one or more RNA molecules, which may be, for example, the exogenous RNA molecule itself or derivatives or modified versions thereof, single-stranded or double-stranded. The exogenous RNA molecule may have such nucleotides as, but not limited to deoxyribonucleotides, ribonucleosides, phosphodiester linkages, modified linkages or bases other than the five biologically occurring bases (adenine, guanine, thymine, cytosine and uracil).

According to some embodiments, the exogenous RNA molecule can be prepared by any method known in the art for the synthesis of RNA molecules. For example, the exogenous RNA molecule may be chemically synthesized using commercially available reagents and synthesizers by methods that are well known in the art. Alternatively, the exogenous RNA molecule can be generated by in vitro and in vivo transcription of DNA sequences encoding the exogenous RNA molecule. Such DNA sequences can be incorporated into a wide variety of vectors which incorporate suitable RNA polymerase promoters such as the T7 or SP6 polymerase promoters. The exogenous RNA molecule may be produced in high yield via in vitro transcription using plasmids such as SPS65. In addition, RNA amplification methods such as Q-beta amplification can be utilized to produce the exogenous RNA molecule.

In some embodiments, the exogenous RNA molecule or the DNA molecule that encodes for the exogenous RNA molecule can be modified at the base moiety, sugar moiety, or phosphate backbone, for example, in order to improve stability of the molecule, hybridization, transport into the cell, and the like. In addition, modifications can be made to reduce susceptibility to nuclease degradation. The exogenous RNA molecule or the DNA molecule that encodes for the exogenous RNA molecule may have other appended groups such as peptides (for example, for targeting host cell receptors in vivo), or agents facilitating transport across the cell membrane or the blood-brain barrier, hybridization-triggered cleavage agents or intercalating agents. Various other well known modifications can be introduced as a means of increasing intracellular stability and half-life. Possible modifications include, but are not limited to, the addition of flanking sequences of ribo- or deoxy-nucleotides to the 5′ and/or 3′ ends of the molecule. In some circumstances where increased stability is desired, nucleic acids having modified internucleoside linkages such as 2′-O-methylation may be preferred. Nucleic acids containing modified internucleoside linkages may be synthesized using reagents and methods that are well known in the art.

According to further embodiments, the exogenous RNA molecule or the DNA molecule that encodes for the exogenous RNA molecule may be purified by any suitable means, as are well known in the art (such as, for example, reverse phase chromatography or gel electrophoresis).

In some embodiments, cells that produce viral vectors that encode for the exogenous RNA, may also be used for transplantation in a body of a patient for continuous treatment. These cells can carry a specific gene that can induce their death in the presence of a specific molecule in the blood (for example, HSV1 Thymidine kinase/Ganciclovir).

In some embodiments, the exogenous RNA molecule may be an RNA molecule or a reproducing RNA molecule. The reproducing RNA molecule is an RNA molecule that comprises a sequence that is complementary to the exogenous RNA molecule such that the reproducing RNA molecule is capable of being replicated in the cell for the formation of the exogenous RNA molecule.

### 6. Uses and Administration of the Composition of the Invention

According to some embodiments, the composition of the present invention may have a variety of different applications including, for example, but not limited to: regulation of gene expression; targeted cell death, treatment of various conditions and disorders, such as, for example: treatment of proliferative disorders such as cancer, treatment of infectious diseases such as HIV, formation of transgenic organisms, suicide gene therapy, and the like. The composition may be used on various organisms, such as, for example, mammals (such as human, murine), avian, plants, and the like. The composition may be used on various cells (in culture and/or in vivo), tissues, organs, and/or on an organism body.

In some embodiments, the composition of the present invention can be used to express and/or activate toxic gene in cells that express a specific endogenous miRNA which is a viral miRNA, for the killing of cancer cells that express this viral miRNA or for killing viral infected cells. In another embodiment of the invention, the composition of the present invention can be used to express and/or activate toxic gene in cells that comprise an oncogenic miRNA (miRNA that is strongly upregulated in cancer cells) as the specific endogenous miRNA, for the killing of these cell.

In some embodiments, the composition of the present invention can be used to express and/or activate reporter gene in the presence of viral or oncogenic miRNA for the diagnosis of diseases like viral infection or cancer. In another embodiment, cells that are stably transfected with vector that encodes for the exogenous RNA molecule can be used for the formation of transgenic organism that is resistant to viral infection or cancer. In another embodiment, the composition of the present invention can be used to stably transfect cells for the formation of transgenic organism that is able to activate reporter gene in the presence of viral miRNA for the diagnosis of viral infection diseases. In yet another embodiment, the composition of the invention can be used to monitor, in real time, the function of miRNAs in the cell and for diagnosis of diseases that involve the formation or the upregulation of miRNAs in the cell (such as, cancer and viral infection).

According to some embodiments, various delivery systems are known and can be used to transfer the composition of the invention into cells, such as, for example, encapsulation in liposomes, microparticles, microcapsules, recombinant cells that are capable of expressing the composition, receptor-mediated endocytosis, construction of the composition of the invention as part of a viral vector or other vector, viral vectors that are capable of being reproduced without killing the cell during the process of reproduction and that comprise the composition of the invention, viral vectors that are not capable of reproduction and that comprise the composition of the invention, injection of cells that produce viral vectors that comprise the composition of the invention, injection of DNA, electroporation, calcium phosphate mediated transfection, and the like, or any other methods known in the art or to be developed in the future.

According to some embodiments, and without wishing to be bound to theory or mechanism, the composition and methods of the present invention may provide a specific and targeted “all or none” response in a cell. In other words, compositions and methods of the present invention are such that the exogenous RNA molecule is cleaved (and consequently, the exogenous protein of interest is expressed and activated) only in target cells, which include a specific endogenous miRNA, whereas cells that do not include the endogenous miRNA will not be effected by the composition of the invention. The composition and methods of the present invention may thus provide enhanced safety and control, since no leakiness of expression of the exogenous protein of interest is observed in cells which do not include the endogenous miRNA

According to some embodiments, there is provided a method for killing a specific cell population, wherein the cell population comprises an endogenous specific endogenous miRNA, which is unique and specific for these cells; the method includes introducing the cells with the composition of the invention, wherein the composition comprises one or more polynucleotides for directing expression of an exogenous protein of interest only in a cell expressing a specific endogenous miRNA, wherein the one or more polynucleotides include or encode for an exogenous RNA molecule, which comprises: a sequence encoding for the exogenous protein of interest; an inhibitory sequence that is capable of inhibiting the expression of the exogenous protein of interest; and a binding site for said specific endogenous miRNA.

According to some embodiments, the exogenous protein of interest may be any type of protein that can damage the cell function and as a result lead to the death of the cell. The protein may be selected from such types of proteins as, but not limited to: toxins, cell growth inhibitors, modulators of cellular growth, inhibitors of cellular signaling pathways, modulators of cellular signaling pathways, modulators of cell permeability, modulators of cellular processes, and the like.

According to some embodiments, there is provided a vector, such as, for example an expression vector (viral vector or non viral vector), which includes one or more polynucleotide sequences encoding for the exogenous RNA molecule, wherein said exogenous RNA molecule includes a sequence encoding for an exogenous protein of interest; an inhibitory sequence that is capable of inhibiting the expression of the exogenous protein of interest; and a binding site for a specific endogenous miRNA. The binding site for the specific endogenous miRNA is of sufficient complementarity to a sequence within a specific endogenous miRNA for the specific endogenous miRNA to direct cleavage of the exogenous RNA molecule at the cleavage site, when the vector is introduced into a cell comprising the specific endogenous miRNA. The cleavage site may be located within the binding site for the specific endogenous miRNA, and further, the cleavage site is located between the inhibitory sequence and the sequence encoding the exogenous protein of interest. In some embodiments, the one or more polynucleotide sequences are DNA sequences. In some embodiments, the one or more polynucleotide sequences are RNA sequence. As known in the art, the vector may further comprise various other polynucleotide sequences that are required for its operation (such as, for example, regulatory sequences, non coding sequences, structural sequences, and the like).

According to further embodiments, the present invention also provides for pharmaceutical compositions comprising an effective amount of the composition of the invention and a pharmaceutically acceptable carrier. The term “Pharmaceutically acceptable” means approved by a regulatory agency of the Federal or a state government or listed in the U.S. Pharmacopeia or other generally recognized pharmacopeia for use in animals, and more particularly in humans. The term “Carrier” refers to a diluent, adjuvant, excipient, or vehicle with which the therapeutic is administered.

According to some embodiments, the pharmaceutical composition may be administered to a subject in need by any administration route known, such as, for example but not limited to: enteral, parenteral, injection, topical, and the like. In some embodiments, it may be desirable to administer the pharmaceutical compositions of the invention locally to a target area in need of treatment. This may be achieved by, for example, and not limited to: local infusion during surgery, topical application, (for example, in conjunction with a wound dressing after surgery), by injection, by means of a catheter, by means of a suppository, or by means of an implant, said implant being of a porous, non-porous, or gelatinous material, including membranes, such as sialastic membranes, or fibers. The local administration may be also achieved by control release drug delivery systems, such as nanoparticles, matrices such as controlled-release polymers or hydrogels.

In some embodiments, the composition of the invention may be administered in amounts which are effective to produce the desired effect in the targeted cell/tissue. Effective dosages of the composition of the invention may be determined through procedures well known to these in the art which address such parameters as biological half-life, bioavailability and toxicity. The amount of the composition of the invention which is effective, depends on the nature of the disease or disorder being treated, and can be determined by standard clinical techniques. In addition, in vitro assays may optionally be employed to help identify optimal dosage ranges. The administered means may also include, but are not limited to permanent or continuous injection of the composition of the invention to the patient blood stream.

In some embodiments, the composition and the pharmaceutical composition comprising same may be administered to various organism, such as, for example, mammals, avian, plants, and the like. For example, the composition and the pharmaceutical composition comprising same may be administered to humans, and animals.

In further embodiments, the present invention also provides a pharmaceutical pack or kit comprising one or more containers filled with one or more of the ingredients of the pharmaceutical compositions of the invention optionally associated with such container(s) can be a notice in the form prescribed by a governmental agency regulating the manufacture, use or sale of pharmaceuticals or biological products, which notice reflects approval by the agency of manufacture, use or sale for human or animal administration.

### EXAMPLES

The following examples are offered by way of illustration and not by way of limitation and are examples of the best embodiments of the present invention.

### Example 1

### Specific Expression of an Exogenous Protein of Interest Encoded by an Exogenous RNA

### General Protocol for Experiments Described in Example 1:

The day before transfection about 120,000 of T293 cells per well were seeded in 24 well plate, at the day of transfection each well was cotransfected with:
1. Renila/luciferase plasmid—170 ng of plasmid expressing Renilla luciferase gene & firefly luciferase gene (plasmid E11, Psv40-INTRON-MCS-RLuc-Phsvtk-Fluc, SEQ ID NO: 22 or plasmid E65, Psv40-INTRON-Tsp-TD1-TLacZ-RLuc-PTS-60ATG-Phsvtk-FLuc, SEQ ID NO. 23).
2. Tested plasmid=30 ng of tested plasmid (as detailed below
3. siRNA+ or siRNA−=10 pmole of siRNA double stranded molecule that can induce cleavage (siRNA+) or does not induce cleavage (siRNA−) of the mRNA encoded by the tested plasmid. (detailed below).
The transfection was performed using lipofectamine 2000 transfection reagent (Invitrogen) according to manufacturer protocol. 48 hrs post transfection the Renilla luciferase gene expression was measured using the dual luciferase reported assay kit (Promega) and luminometer (glomax 20/20 promega), and the relative light units (RLU) were determined.

### The Tested Plasmid May be any Type of the Following Plasmids:

Negative control=Plasmid that does not encode for a diphtheria toxin (DTA);
Positive control=Plasmid that constitutively encodes for diphtheria toxin (DTA);
Test plasmid=plasmid of the composition of the invention, i.e. plasmid comprising target sites for siRNA+ between an inhibitory sequence and a downstream sequence encoding for diphtheria toxin (DTA). For the test plasmid, when the co-transfected siRNA+ cleaves the inhibitory sequence of the test plasmid, the diphtheria toxin is capable of being expressed and kills the cells in which it is expressed, thereby—reducing Renilla expression and overall measurement of RLU.
The tested plasmid was tested with 2 different siRNAs+ and with 2 different siRNAs−, separately, and each in triplicate.
The results are calculated as follows:
Fold of Activation=Average of measured RLU (Relative light unit) in the presence of each of the 2 siRNA− with the test plasmid (6 wells) divided by the average of RLU using one of the siRNA+ with the test plasmid (3 wells).
Fold of leakage=Average of RLU using all the siRNAs−/+ with the negative control plasmid divided by the Average of RLU using each of the 2 siRNA− with the test plasmid.
siRNA+/−RLU=Average of measured RLU in the presence of one co-transfected siRNA+ or the presence of two co-transfected siRNA-, independently.

The plasmids were constructed using common and known methods practiced in the art of molecular biology. The backbone vectors for the constructed plasmids described herein below are: psiCHECK™-2 Vectors (promega, Cat. No. C8021) or pcmv6-A-GFP (OriGene, Cat. No. PS100026). The appended name of each plasmid indicates sequences which are comprised within the plasmid sequence, as further detailed below, with respect to the test plasmids.

siRNA Sequences:
1. RL Duplex (Dharmacon, Cat. No. P-002070-01-20) (SEQ ID NO. 65 (sense strand) and SEQ ID 66 (anti sense strand)).
2. GFPDuplex II (Dharmacon, Cat. No. P-002048-02-20), (SEQ ID NO. 67 (sense strand) and SEQ ID NO.68 (anti sense strand)).
3. siRNA—Control (Sigma, Cat. No., VC30002 000010), (SEQ ID NO. 69 (sense strand) and SEQ ID NO.70, (anti sense strand)).
4. Anti βGal siRNA-1 ((target site: Tlacz (SEQ ID NO. 71)), Dharmacon, Cat. No. P-002070-01-20) (SEQ ID NO. 72 (sense strand) and SEQ ID NO. 73 (antisense strand)).
5. Luciferase GL3 Duplex ((target site: Tfluc (SEQ ID NO. 74)), Dharmacon, Cat. No. D-001400-01-20), (SEQ ID NO. 75 (sense strand) and SEQ ID NO. 76 (antisense strand)).
6. GFPDuplex I ((target site: TD1, (SEQ ID NO. 77)), Dharmacon, Cat. No. P-002048-01-20), (SEQ ID NO. 78 (sense strand) and SEQ ID NO. 79 (antisense strand)).
7. TCTL ((target site: TCTL (SEQ ID NO. 80)), SEQ ID NO. 81 (sense strand) and SEQ ID NO. 82 (anti sense strand)).

In each experiment, the siRNA that has target site in the test plasmid is used as siRNA+, and the other siRNAs that do not have a corresponding target site in the tested plasmid was used as siRNA−.

### Negative Control Plasmids:

1. E34 (SEQ ID NO. 10)—Pcmv-4ORF̂-TD1-Tfluc-Psv40-TGFP.
2. E71 (SEQ ID. NO. 17)—Psv40-INTRON-4ORF̂-Phsvtk-Fluc.
3. E38-3CARz-4S&L. The insert of E38 (SEQ ID. NO. 19) was ligated into a PMK shuttle vector (GeneArt) at pad and XhoI restriction sites.

### Positive Control Plasmids:

1. E28 (SEQ ID. NO. 11)—Pcmv-Tfluc-TD1-cDTAWT-Psv40-TGFP.

### 2. E20 (SEQ ID. NO. 12)—Pcmv-nsDTA-Psv40-TGFP

### 3. E70 (SEQ ID. NO. 13)—Psv40-INTRON-cDTAWT-Phsvtk-Fluc

### 4. E3 (SEQ ID. NO. 14)—Pcmv-KDTA-Psv40-TGFP

5. E89 (SEQ ID. NO. 15)—Pcmv-DT̂A-Psv40-TGFP
6. E110 (SEQ ID. NO. 16)—Pcmv-D5̂TA-Psv40-TGFP

### 7. E4 (SEQ ID. NO. 18)—Pcmv-KDTA-Psv40-Hygro

### 8. E10 (SEQ ID. NO. 20)—Pef1-DTA24-ZEO::GFP-Pcmv

9. E143 (SEQ ID. NO. 21)—3PolyA-Prp119-cDTAWT-Phsvtk-Fluc

### Test Plasmids

1. E80 (SEQ ID. NO. 1)—Pcmv-4ORF̂-TD1-Tfluc-S-cDTAWT-Psv40-TGFP (pCMV promoter (nts. 420-938 of SEQ ID NO. 1); 4ORF̂=Inhibitory sequence composed of: 9 TISU sequences and 57 kozak sequences, with 57, 57, 36, 36, 21, 21, 21, and 21 nt between adjacent ATG codons, in 4 consecutive ORFs (nt 1027-3547 of SEQ ID NO. 1). The first ORF (nt. 1031-1651 of SEQ ID NO. 1) is 621 nt & is translated from TISU (nt. 1027-1038 of SEQ ID NO. 1), and the next 3ORF̂ (nt. 1662-2996, nt. 2306-2941 and nt 2951-3547 of SEQ ID NO. 1) are translated from Kozak sequence, The last ORF (nt 2951-3547 of SEQ ID NO. 1) stops before the coding sequence of the wild type DTA (cDTAwt=wt DTA coding sequence, without promoter/splicing/termination/polyA sites and with kozak sequence (nt 3568-4155 of SEQ ID NO. 1); followed by TGFP coding sequence under the control of the SV40 promoter)). The plasmid further comprises target sites TD1 (SEQ ID NO. 77) and Tfluc (SEQ ID NO. 74).
2. E54 (SEQ ID. NO 2)—Pcmv-4CARZ-PTS-60ATĜ-3ORF̂-TD1-Tfluc-incDTAWT-Psv40-TGFP (pCMV promoter (nucleotides (nt.) 420-938 of SEQ ID NO. 2); 4CAR=4 Cis Acting Ribozyme (nt. 1013-1373 of SEQ ID NO. 2); PTS=Peroxisomal targeting signal (nt. 1420-1500 of SEQ ID NO. 2); 60ATĜ=61 ATG, 46 in Kozak sequence with 53 nt between almost every 2 ATG (nt. 1534-4554 of SEQ ID NO. 2) and with stop codons inside the DTA coding sequence (nt. 6745-7332 of SEQ ID NO. 2); TGFP coding sequence (nt. 8452-9143 of SEQ ID NO. 2) under the control of the psv40 promoter (nt. 8092-8399 of SEQ ID. NO. 2)). The plasmid further comprises target sites TD1 (SEQ ID NO. 77) and Tfluc (SEQ ID NO. 74).
3. E113 (SEQ ID. NO. 3)—Pcmv-4ORF̂-TD1-Tfluc-PK-D5̂TA-Psv40-TGFP (pCMV promoter (nts. 420-938 of SEQ ID NO. 3); 4ORF̂ (nt. 1027-3547 of SEQ ID NO. 3); PK=pseudoknot—stem and loop, such that the 6 nt of the loop are hybridized to the start codon of DTA (nt 3561-3611 of SEQ ID No. 3); 5̂=5 human introns (nts. 3712-3801, 3856-3960, 4066-4173, 4380-4519 and 4617-4783 of SEQ ID NO. 3) that are located within the coding sequence of the DTA (nts. 3609-3806 of SEQ ID NO. 3) and contain T-rich sequences for terminating RNA Polymerase 1 and/or 3 transcription, the introns are embedded in cDTAwt coding sequence; TGFP coding sequence (nts 5906-6597 of SEQ ID NO. 3) under the control of the psv40 promoter (nts. 5546-5853 of SEQ ID NO. 3)). The plasmid further comprises target sites TD1 (SEQ ID NO. 77) and Tfluc (SEQ ID NO. 74).
4. E91 (SEQ ID. NO. 4)—Pcmv-4ORF̂-TD1-Tfluc-DT̂A-Psv40-TGFP (pCMV promoter (nts. 420-938 of SEQ ID NO. 4), 4ORF̂ (nt. 1027-3507 of SEQ ID NO. 4); DT̂A=kozak DTA with an intron from Human Collagen 16A1 gene and without promoter/splicing/polyA signal (nt. 3520-4444 of SEQ ID NO. 4); TGFP coding sequence (nt. 5544-6235 of SEQ ID NO. 4) under the control of pSV40 promoter (nt. 5184-5491) The plasmid further comprises target sites TD1 (SEQ ID NO. 77) and Tfluc (SEQ ID NO. 74).
5. E112 (SEQ ID. NO. 5)—Pcmv-4ORF̂-2×TLacZinINTRON-8X[TCTL+TD1]-PK-D5̂TA-Psv40-TGFP (pCMV promoter (nts. 420-938 of SEQ ID NO. 5), 4ORF̂ (nt. 1027-3436 of SEQ ID NO. 5); 2×TLacZinINTRON=2 target of TLacZ in the intron of the commercial plasmid pSELECT-GFPzeo-LacZ (nt. 3438-3638 of SEQ ID NO. 5); 8X[TCTL+TD1] (nt. 3647-4052 of SEQ ID NO. 5); PK=pseudoknot—stem and loop, such that the 6 nt of the loop are hybridized to the start codon of DTA (nt 4059-4109 of SEQ ID No. 5); 5̂=5 human introns (nts. 4210-4299, 4354-4458, 4564-4671, 4878-5017 and 5115-5281 of SEQ ID NO. 5) that are located within the coding sequence of the DTA (nt. 4107-5304 of SEQ ID NO. 5) and contain T-rich sequences for terminating RNA Polymerase 1 and/or 3 transcription, the introns are embedded in a cDTAwt coding sequence; TGFP coding sequence (nt 6404-7095 of SEQ ID NO. 5) under the control of the psv40 promoter (nts. 6044-6351 of SEQ ID NO. 5)). The plasmid further comprises 8 copies of target sites TD1 (SEQ ID NO. 77), TCTL (SEQ ID NO. 80) and 2 copies of TLacZ (SEQ ID NO. 71).
6. E87 (SEQ ID. NO. 6)-Pcmv-4ORF̂-TD1-3TLacZ-Tctl-BGlob-25G-XRN1S&L-DT̂A-Psv40-TGFP (pCMV promoter (nts. 420-938 of SEQ ID NO. 6); 4ORF̂ (nt. 1027-3430 of SEQ ID NO. 6); BGlob=beta globin 5′ truncated end that is capped (nt. 3577-3655 of SEQ ID NO 6). 25G=a stretch of 25 consecutive G nucleotides (nt. 3660-3684 of SEQ ID NO. 6) that can block/interfere with XRN exoribonuclease enzyme; XRN1S&L=stem and loop structure of the yellow fever virus 3′UTR that can block XRN1 exoribonuclease (nt. 3687-3767 of SEQ ID. NO. 6). DT̂A=kozak DTA with an intron from Human Collagen 16A1 gene and without promoter/splicing/polyA signal (nt. 3787-4711 of SEQ ID NO. 6); TGFP coding sequence (nt 6404-7095 of SEQ ID NO. 6) under the control of the psv40 promoter (nts. 5811-6502 of SEQ ID NO. 6)). The plasmid further comprises TD1 (SEQ ID NO. 77), 3 copies of TLacz (SEQ ID NO. 71) and TCTL target sites (SEQ ID NO. 80).
7. E123 (SEQ ID. NO. 7)—Psv40-INTRON-4ORF̂-3X[TD1-TLacZ]-4PTE-SV40intron-HBB-DTA-Phsvtk-Fluc (pSV40 promoter (nt. 7-419 of SEQ ID NO. 7), 4ORF̂=9 TISU sequences and 57 kozak sequences, with 57, 57, 36, 36, 21, 21, 21, and 21 nt between adjacent ATG codons, in 4 consecutive ORFs (nt 722-2387 of SEQ ID NO. 7); 4PTE=4 kinds of the stem and loop structures of the Palindromic termination element (nt. 3318-3473 of SEQ ID NO. 7). SV40intron ═SV40 small t antigen intron (nt. 3505-3596 of SEQ ID NO. 7); HBB=hemoglobin beta mRNA without ATG and including its first intron (nt. 3627-4406 of SEQ ID NO. 7); cDTAwt coding sequence (nt. 4431-5014 of SEQ ID NO. 7); HSKVK promoter (nt. 5106-5858 of SEQ ID NO. 7) and firefly luciferase coding sequence (nt. 5894-7546 of SEQ ID. NO. 7). The plasmid further comprises 3 copies of TD1 (SEQ ID NO. 77) and TLacz target sites (SEQ ID NO. 71).
8. E30 (SEQ ID. NO. 8)—Pcmv-4ORF̂-TD1-Tfluc-incDTAWT-Psv40-TGFP (pCMV promoter (nts. 420-938 of SEQ ID NO. 8); 4ORF̂=9 TISU sequences and 57 kozak sequences, with 57, 57, 36, 36, 21, 21, 21, and 21 nt between adjacent ATG codons, in 4 consecutive ORFs (nt 1027-3547 of SEQ ID NO. 8). The first ORF (nt. 1031-1651 of SEQ ID NO. 8) is translated from TISU (nt. 1027-1038 of SEQ ID NO. 8), and the next 3ORF̂ (nt. 1662-2996, nt. 2306-2941 and nt 2951-3547 of SEQ ID NO. 8) are translated from Kozak sequence, The last ORF (nt 2951-3516 of SEQ ID NO. 8) stops inside the coding sequence of the wild type DTA (cDTAwt=wt DTA coding region, without promoter/splicing/termination/polyA sites and with kozak sequence (nt 3568-4155 of SEQ ID NO. 8); followed by TGFP coding sequence under the control of the SV40 promoter)). The plasmid further comprises target sites TD1 (SEQ ID NO. 77) and Tfluc (SEQ ID NO. 74).
9. E142 (SEQ ID. NO. 9)—3PolyA-Prp119-4ORF̂-TD1-Tfluc-S-cDTAWT-Phsvtk-Fluc. 3PolyA=HSV poly A, SV40 poly A, synthetic poly A (nt. 60-247 of SEQ ID NO. 9); Prp119=promoter of RPL19 (ribosomal protein L19) taken with its first intron (nt. 248-1941 of SEQ ID NO. 9); 4ORF̂=9 TISU sequences and 57 kozak sequences, with 57, 57, 36, 36, 21, 21, 21, and 21 nt between adjacent ATG codons, in 4 consecutive ORFs (nt 1948-4366 of SEQ ID NO. 9); coding sequence of the wild type DTA (nt. 4457-5044 of SEQ ID NO. 9); HSKVK promoter (nt. 5136-5888 of SEQ ID NO. 9) and firefly luciferase coding sequence (nt. 5924-7576 of SEQ ID. NO. 9). The plasmid further comprises target sites TD1 (SEQ ID NO. 77) and Tfluc (SEQ ID NO. 74).

### Results:

The results are presented in following tables 1-5 and 6A-C. The results show the RLU measured in cells transfected with the indicated plasmids and siRNA molecules under various experimental conditions. The siRNA+ molecules used are the siRNA molecules that can bind their corresponding target sequence(s) within the tested plasmid.

TABLE 1

| | | | RLU in the | RLU in the |
|---|---|---|---|---|
| | Fold of | Fold of | presence of | presence of |
| Tested plasmid | Activation | leakage | siRNA+ | siRNA− |
| E34 (SEQ ID NO. 10) - Pcmv-4ORF\hat{ }-TD1-Tfluc--- | | | | 93M |
| Psv40-TGFP | | | | |
| E28 (SEQ ID NO. 11) - Pcmv-Tfluc-TD1- | | | | 35K |
| cDTAWT---Psv40-TGFP | | | | |
| E20 (SEQ ID NO. 12) - Pcmv-nsDTA---Psv40- | | | | 52K |
| TGFP | | | | |
| E70 (SEQ ID NO. 13) - Psv40-INTRON-cDTAWT--- | | | | 249K |
| Phsvtk-Fluc | | | | |
| E54 (SEQ ID. NO. 2) - Pcmv-4CARZ-PTS- | 4 | 5.1 | 4.4M | 18M |
| 60ATG\hat{ }-3ORF\hat{ }-TD1-Tfluc-incDTAWT---Psv40- | | | | |
| TGFP | | | | |

TABLE 2

| | | | RLU in the | RLU in the |
|---|---|---|---|---|
| | Fold of | Fold of | presence of | presence of |
| Tested plasmid | Activation | leakage | siRNA+ | siRNA− |
| E34 (SEQ ID NO. 10)- Pcmv-4ORF\hat{ }-TD1-Tfluc--- | | | | 33M |
| Psv40-TGFP | | | | |
| E28 (SEQ ID NO. 11) - Pcmv-Tfluc-TD1- | | | | 33K |
| cDTAWT---Psv40-TGFP | | | | |
| E3 (SEQ ID NO. 14) - Pcmv-KDTA---Psv40- | | | | 45K |
| TGFP | | | | |
| E89 (SEQ ID NO. 15) - Pcmv---DT\hat{ }A---Psv40- | | | | 16K |
| TGFP | | | | |
| E110 (SEQ ID NO. 16) - Pcmv-D5\hat{ }TA---Psv40- | | | | 21K |
| TGFP | | | | |
| E113 (SEQ ID. NO. 3) - Pcmv-4ORF\hat{ }-TD1-Tfluc- | 6 | 15 | 367K | 2.2M |
| PK-D5\hat{ }TA---Psv40-TGFP | | | | |
| E80 (SEQ ID. NO. 1) - Pcmv-4ORF\hat{ }-TD1-Tfluc- | 5.2 | 15 | 427K | 2.2M |
| S-cDTAWT---Psv40-TGFP | | | | |
| E91 (SEQ ID. NO. 4) - Pcmv-4ORF\hat{ }-TD1-Tfluc- | 4.73 | 15 | 467K | 2.2M |
| DT\hat{ }A---Psv40 TGFP | | | | |
| E112 (SEQ ID. NO. 5) - Pcmv-4ORF\hat{ }- | 4.25 | 18.3 | 425K | 1.8M |
| 2xTLacZinINTRON-8X[TCTL + TD1]-PK- | | | | |
| D5\hat{ }TA---Psv40-TGFP | | | | |
| E87 (SEQ ID. NO. 6) - Pcmv-4ORF\hat{ }-TD1- | 4.15 | 22 | 364K | 1.5M |
| 3TLacZ-Tctl-BGlob-25G-XRN1S&L-DT\hat{ }A--- | | | | |
| Psv40-TGFP | | | | |

TABLE 3

| | | | RLU in the | RLU in the |
|---|---|---|---|---|
| | Fold of | Fold of | presence of | presence of |
| Tested plasmid | Activation | leakage | siRNA+ | siRNA− |
| E71 (SEQ ID NO. 17) - Psv40-INTRON- | | | | 22.5M |
| 4ORF\hat{ }---Phsvtk-Fluc | | | | |
| E70 (SEQ ID NO. 3) - Psv40-INTRON- | | | | 819K |
| cDTAWT---Phsvtk-Fluc | | | | |
| E123 (SEQ ID. NO. 7) - Psv40-INTRON-4ORF\hat{ }- | 3.37 | 1.8 | 3.7M | 12.5M |
| 3X[TD1 − TLacZ]-4PTE-SV40intron-HBB-DTA--- | | | | |
| Phsvtk-Fluc | | | | |

TABLE 4

| | | | RLU in the | RLU in the |
|---|---|---|---|---|
| | Fold of | Fold of | presence of | presence of |
| Tested plasmid | Activation | leakage | siRNA+ | siRNA− |
| E34 (SEQ ID NO. 10) - Pcmv-4ORF\hat{ }-TD1- | | | | 35M |
| Tfluc---Psv40-TGFP | | | | |
| E3 (SEQ ID NO. 14) - Pcmv-KDTA---Psv40-TGFP | | | | 47K |
| E4 (SEQ ID NO. 18) - Pcmv-KDTA---Psv40-Hygro | | | | 54K |
| E30 (SEQ ID. NO. 8) - Pcmv-4ORF\hat{ }-TD1- | 2.96 | 10.9 | 1.1M | 3.2M |
| Tfluc-incDTAWT---Psv40-TGFP | | | | |

TABLE 5:

| | | | RLU in the | RLU in the |
|---|---|---|---|---|
| | Fold of | Fold of | presence of | presence of |
| Tested plasmid | Activation | leakage | siRNA+ | siRNA− |
| E38 (SEQ ID NO. 19) - 3CARz-4S&L | | | | 137M |
| E10 (SEQ ID NO. 20) - Pefl-DTA24--- | | | | 55K |
| ZEO::GFP-Pcmv | | | | |
| E143 (SEQ ID NO. 21) - 3PolyA-Prpl19- | | | | 132K |
| cDAWT---Phsvtk-Fluc | | | | |
| E142 (SEQ ID. NO. 9) - 3PolyA-Prpl19-4ORF\hat{ }- | 2.53 | 5.9 | 9.1M | 23M |
| TD1-Tfluc-S-cDTAWT---Phsvtk-Fluc | | | | |

TABLE 6A

| | | Experiment number | | | | | | |
|---|---|---|---|---|---|---|---|---|
| | | 1 | 2 | 3 | 4 | 5 | 6 | 7 |
| Number of | #293cells | 135K | 180K | 150K | 120K | 150K | 120K | 90K |
| 293HEK cells | | | | | | | | |
| per well (24 | | | | | | | | |
| well plate) | | | | | | | | |
| Hours post | hrPT | 5 hr | 9 hr | 48 hr | 48 hr | 48 hr | 48 hr | 48 hr |
| transfection | | | | | | | | |
| co-transfection | REN | E11[170] | E11[170] | E11[170] | E11[170] | E11[170] | E11[170] | E11[170] |
| of Renilla | | | | | | | | |
| expressing | | | | | | | | |
| plasmid [ng] | | | | | | | | |
| co-transfection | siRNA | [10] | [10] | [10] | [10] | [10] | [10] | [10] |
| of siRNA+ or | | | | | | | | |
| siRNA−: [pico | | | | | | | | |
| mole] | | | | | | | | |
| co-transfection | ↓/RLU | [30] | [30] | [30] | [30] | [30] | [30] | [30] |
| of one of the | | | | | | | | |
| test plasmids | | | | | | | | |
| below [ng]:/ | | | | | | | | |
| Results shown | | | | | | | | |
| below for each | | | | | | | | |
| plasmid are | | | | | | | | |
| RLU measured | | | | | | | | |
| under the | | | | | | | | |
| indicated | | | | | | | | |
| experimental | | | | | | | | |
| condition | | | | | | | | |
| Co transfection | E28 | 8.38K | 37.89K | 81.5K | 33K | 30.6K | 9.8K | 7.59K |
| of a Plasmid | (SEQ ID | | | | | | | |
| comprising the | NO. 11) | | | | | | | |
| sequence: | | | | | | | | |
| Pcmv-Tfluc- | | | | | | | | |
| TD1- | | | | | | | | |
| cDTAWT--- | | | | | | | | |
| Psv40-TGFP. | | | | | | | | |
| Co transfection | E34 | 161K | 8.8M | 83M | 33M | 40M | 23M | 11M |
| of Plasmid | (SEQ ID | | | | | | | |
| comprising the | NO. 10) | | | | | | | |
| sequence: | | | | | | | | |
| Pcmv-4ORF\hat{ }- | | | | | | | | |
| TD1-Tfluc--- | | | | | | | | |
| Psv40-TGFP | | | | | | | | |
| Co transfection | E80 | 110K | 4.15M | 7.17M | 2.2M | 4.33M | 2.3M | 1.1M |
| of Plasmid | (SEQ ID. | | | | | | | |
| comprising the | NO. 1) | | | | | | | |
| sequence: | | | | | | | | |
| Pcmv-4ORF\hat{ }- | | | | | | | | |
| TD1-Tfluc-S- | | | | | | | | |
| cDTAWT--- | | | | | | | | |
| Psv40-TGFP + | | | | | | | | |
| co-transfected | | | | | | | | |
| with siRNA− | | | | | | | | |
| Co transfection | E80 | 33K* | 1.35M* | 3M* | 427K* | 1.65M* | 800K | 354K |
| of Plasmid | (SEQ ID. | | | | | | | |
| comprising the | NO. 1) | | | | | | | |
| sequence | | | | | | | | |
| Pcmv-4ORF\hat{ }- | | | | | | | | |
| TD1-Tfluc-S- | | | | | | | | |
| cDTAWT--- | | | | | | | | |
| Psv40-TGFP | | | | | | | | |
| co-transfected | | | | | | | | |
| with siRNA+ | | | | | | | | |
| Fold of | si−/si+ | 3.33 | 3 | 2.4 | 5.1 | 2.6 | 2.87 | 3.1 |
| activation = | | | | | | | | |
| RLU measured | | | | | | | | |
| in the presence | | | | | | | | |
| of siRNA− | | | | | | | | |
| divided by RLU | | | | | | | | |
| measured in the | | | | | | | | |
| presence of | | | | | | | | |
| siRNA+ | | | | | | | | |
| Fold of | E34/ | 1.46 | 2.12 | 11.57 | 15 | 9.23 | 10 | 10 |
| leakiness = E34 | E80− | | | | | | | |
| (SEQ ID NO./ | | | | | | | | |
| E80− {smaller | | | | | | | | |
| than 1 = 0 | | | | | | | | |
| leakage} | | | | | | | | |

TABLE 6B

| | | Experiment number | | | | | | |
|---|---|---|---|---|---|---|---|---|
| | | 8 | 9 | 10 | 11 | 12 | 13 | 14 |
| Number of | #293cells | 100K | 120K | 120K | 100K | 100K | 100K | 125K |
| 293HEK cells | | | | | | | | |
| per well (24 well | | | | | | | | |
| plate) | | | | | | | | |
| Hours post | hrPT | 72 hr | 48 hr | 48 hr | 48 hr | 48 hr | 48 hr | 48 hr |
| transfection | | | | | | | | |
| co-transfection | REN | E11[195] | E65[15] | E11[170] | E11[170] | E11[140] | E11[110] | E11[170] |
| of Renilla | | | | | | | | |
| expressing | | | | | | | | |
| plasmid [ng] | | | | | | | | |
| co-transfection | siRNA | [10] | [10] | [5.5] | [10] | [10] | [10] | [10] |
| of siRNA+ or | | | | | | | | |
| siRNA− | | | | | | | | |
| [picomole] | | | | | | | | |
| co-transfection | ↓/RLU | [5] | [30]** | [30] | [30] | [60] | [90] | [30] |
| of one of the test | | | | | | | | |
| plasmids below | | | | | | | | |
| [ng]:/results | | | | | | | | |
| shown are RLU | | | | | | | | |
| under the | | | | | | | | |
| indicated | | | | | | | | |
| experimental | | | | | | | | |
| condition | | | | | | | | |
| Co transfection | E28 | 128K | 2.43K | | | | | |
| of Plasmid | (SEQ ID | | | | | | | |
| comprising the | NO. 11) | | | | | | | |
| sequence: Pcmv- | | | | | | | | |
| Tfluc-TD1- | | | | | | | | |
| cDTAWT--- | | | | | | | | |
| Psv40-TGFP | | | | | | | | |
| Co transfection | E34 | 117M | 1.1M | 97M | | | | |
| of Plasmid | (SEQ ID | | | | | | | |
| comprising the | NO. 10) | | | | | | | |
| sequence: Pcmv- | | | | | | | | |
| 4ORF\hat{ }-TD1- | | | | | | | | |
| Tfluc---Psv40- | | | | | | | | |
| TGFP | | | | | | | | |
| Co transfection | E80 | 14M | 65K | 10.3M | 4.9M | 2.4M | 1.4M | 7.2M |
| of Plasmid | (SEQ ID. | | | | | | | |
| comprising the | NO. 1) | | | | | | | |
| sequence: Pcmv- | | | | | | | | |
| 4ORF\hat{ }-TD1- | | | | | | | | |
| Tfluc-S- | | | | | | | | |
| cDTAWT--- | | | | | | | | |
| Psv40-TGFP + | | | | | | | | |
| co-transfected | | | | | | | | |
| with siRNA− | | | | | | | | |
| Co transfection | E80 | 2.69M* | 18K* | 2.7M* | 1.2M* | 586K* | 347.K* | 2.1M* |
| of Plasmid | (SEQ ID. | | | | | | | |
| comprising the | NO. 1) | | | | | | | |
| sequence Pcmv- | | | | | | | | |
| 4ORF\hat{ }-TD1- | | | | | | | | |
| Tfluc-S- | | | | | | | | |
| cDTAWT- | | | | | | | | |
| Psv40-TGFP co- | | | | | | | | |
| transfected with | | | | | | | | |
| siRNA+ | | | | | | | | |
| Fold of | si−/si+ | 5.2 | 3.6 | 3.8 | 4 | 4.1 | 4 | 3.4 |
| activation = | | | | | | | | |
| RLU measured | | | | | | | | |
| in the presence | | | | | | | | |
| of siRNA− | | | | | | | | |
| divided by RLU | | | | | | | | |
| measured in the | | | | | | | | |
| presence of | | | | | | | | |
| siRNA+ | | | | | | | | |
| Fold of leakiness = | E34/ | 8.35 | 16.92 | 9.41 | | | | |
| E34 (SEQ ID | E80− | | | | | | | |
| NO./E80− | | | | | | | | |
| {smaller than 1 = | | | | | | | | |
| 0 leakage} | | | | | | | | |

TABLE 6C

| | | Experiment number | | | | | | |
|---|---|---|---|---|---|---|---|---|
| | | 15 | 16 | 17 | 18 | 19 | 20 | 21 |
| Number of | #293cells | 125K | 125K | 100K | 100K | 100K | 100K | 200K |
| 293HEK cells | | | | | | | | |
| per well (24 well | | | | | | | | |
| plate) | | | | | | | | |
| Hours post | hrPT | 48 hr | 48 hr | 72 hr | 72 hr | 72 hr | 72 hr | 24 hr |
| transfection | | | | | | | | |
| co-transfection | REN | E11[140] | E11[110] | E11[150] | E11[150] | E11[750] | E11[750] | E11[170] |
| of Renilla | | | | | | | | |
| expressing | | | | | | | | |
| plasmid [ng] | | | | | | | | |
| co-transfection | siRNA | [10] | [10] | [10] | [15] | [10] | [15] | [10] |
| of siRNA+ or | | | | | | | | |
| siRNA−: [pico | | | | | | | | |
| mole] | | | | | | | | |
| co-transfection | ↓/RLU | [60] | [90] | [50] | [50] | [50] | [50] | [30] |
| of one of the test | | | | | | | | |
| plasmids below | | | | | | | | |
| [ng]:/results | | | | | | | | |
| shown are RLU | | | | | | | | |
| under the | | | | | | | | |
| indicated | | | | | | | | |
| experimental | | | | | | | | |
| condition | | | | | | | | |
| Co transfection | E28 | | | | | | | 97K |
| of Plasmid | (SEQ ID | | | | | | | |
| comprising the | NO. 11) | | | | | | | |
| sequence: Pcmv- | | | | | | | | |
| Tfluc-TD1- | | | | | | | | |
| cDTAWT--- | | | | | | | | |
| Psv40-TGFP | | | | | | | | |
| Co transfection | E34 | | | | | | | 10.7M |
| of Plasmid | (SEQ ID | | | | | | | |
| comprising the | NO. 10) | | | | | | | |
| sequence: Pcmv- | | | | | | | | |
| 4ORF\hat{ }-TD1- | | | | | | | | |
| Tfluc---Psv40- | | | | | | | | |
| TGFP | | | | | | | | |
| Co transfection | E80 | 3.16M | 1.76M | 3.67M | 4.3M | 13.3M | 13.3M | 4.2M |
| of Plasmid | (SEQ ID. | | | | | | | |
| comprising the | NO. 1) | | | | | | | |
| sequence: Pcmv- | | | | | | | | |
| 4ORF\hat{ }-TD1- | | | | | | | | |
| Tfluc-S- | | | | | | | | |
| cDTAWT--- | | | | | | | | |
| Psv40-TGFP + | | | | | | | | |
| co-transfected | | | | | | | | |
| with siRNA− | | | | | | | | |
| Co transfection | E80 | 950K* | 573K* | 1.4M* | 1.4M* | 5.8M* | 6.1M* | 2.1M* |
| of Plasmid | (SEQ ID. | | | | | | | |
| comprising the | NO. 1) | | | | | | | |
| sequence Pcmv- | | | | | | | | |
| 4ORF\hat{ }-TD1- | | | | | | | | |
| Tfluc-S- | | | | | | | | |
| cDTAWT--- | | | | | | | | |
| Psv40-TGFP co- | | | | | | | | |
| transfected with | | | | | | | | |
| siRNA+ | | | | | | | | |
| Fold of | si−/si+ | 3.32 | 3 | 2.6 | 3 | 2.3 | 2.18 | 2 |
| activation = | | | | | | | | |
| RLU measured | | | | | | | | |
| in the presence | | | | | | | | |
| of siRNA− | | | | | | | | |
| divided by RLU | | | | | | | | |
| measured in the | | | | | | | | |
| presence of | | | | | | | | |
| siRNA+ | | | | | | | | |
| Fold of leakiness = | E34/ | | | | | | | 2.54 |
| E34 (SEQ ID | E80− | | | | | | | |
| NO./E80− | | | | | | | | |
| {smaller than 1 = | | | | | | | | |
| 0 leakage} | | | | | | | | |

With respect to Table 6A-6C:
*= Indicate that the 2 siRNA+ show significant activation;
**= co-transfected also with 155 ng of plasmid E38 (SEQ ID NO. 19).

The results presented above in Tables 1-5 and 6A-6C clearly show that in the presence of an siRNA molecule(s) capable of inducing cleavage of the exogenous RNA of interest, the exogenous protein of interest (DTA) is expressed which, in turn results in increased cell death. The increased cell death results in reduced overall RLU measurements in the well, since less cells are expressing/producing the luciferase gene. The results demonstrate that indeed, only in cells which comprise a specific siRNA, the exogenous protein of interest (DTA in this example) is expressed, since only in these cells, cleavage of the exogenous RNA of interest at the cleavage site is induced, thereby allowing expression of the exogenous protein of interest in the cells.

### Example 2

### Use of the Composition of the Invention to Kill EBV-Associated Gastric Carcinomas Cancer Cells, Nasopharyngeal Carcinoma Cancer Cells and Burkitt's Lymphoma Cancer Cells

Gastric carcinoma is the most common cancer in the world after lung cancer and is a major cause of mortality and morbidity. 5-year survival rates are less than 20%. About 6 to 16% of gastric carcinoma cases worldwide are associated with Epstein-Barr virus (EBV) that found in almost all tumor cells [21]. Burkitt's lymphoma is a type of Non-Hodgkin's lymphoma commonly affects the jaw bone, forming a huge tumor mass. B cell immortalized by EBV is the first step that eventually leads to Burkitt's lymphoma. Nasopharyngeal carcinoma is a cancer found in the upper respiratory tract, most commonly in the nasopharynx, and is strongly linked to the EBV virus.

Post-Transplant Lymphoproliferative Disorder (PTLPD) is another B cell lymphoma that arises in immuno-compromised patients such as those with AIDS or who have undergone organ transplantation with associated immunosuppression, and thus it is postulated to be linked to EBV. Smooth muscle tumors in malignant patients and Hodgkin's lymphoma are also associated with EBV.

In the United States, as many as 95% of adults between 35 and 40 years of age have been infected with Epstein-Barr Virus (EBV or HHV-4).

Epstein-Barr virus encodes 23 miRNAs that function in regulation of tumor and in suppression of apoptosis [13]. Multiple miRNAs have been identified within two genomic regions of the Epstein-Barr virus and are expressed during latent infection of transformed B cell lines [20].

Expression of the EBV miRNA miR-BART1 (SEQ ID NO. 41) was observed in B cells Burkitt's lymphoma, nasopharyngeal carcinoma cells infected with EBV and EBV-associated gastric carcinomas (EBVaGCs) [21]. Thus these cancers can be killed by using the composition of the invention to kill cells that express miR-BART1.

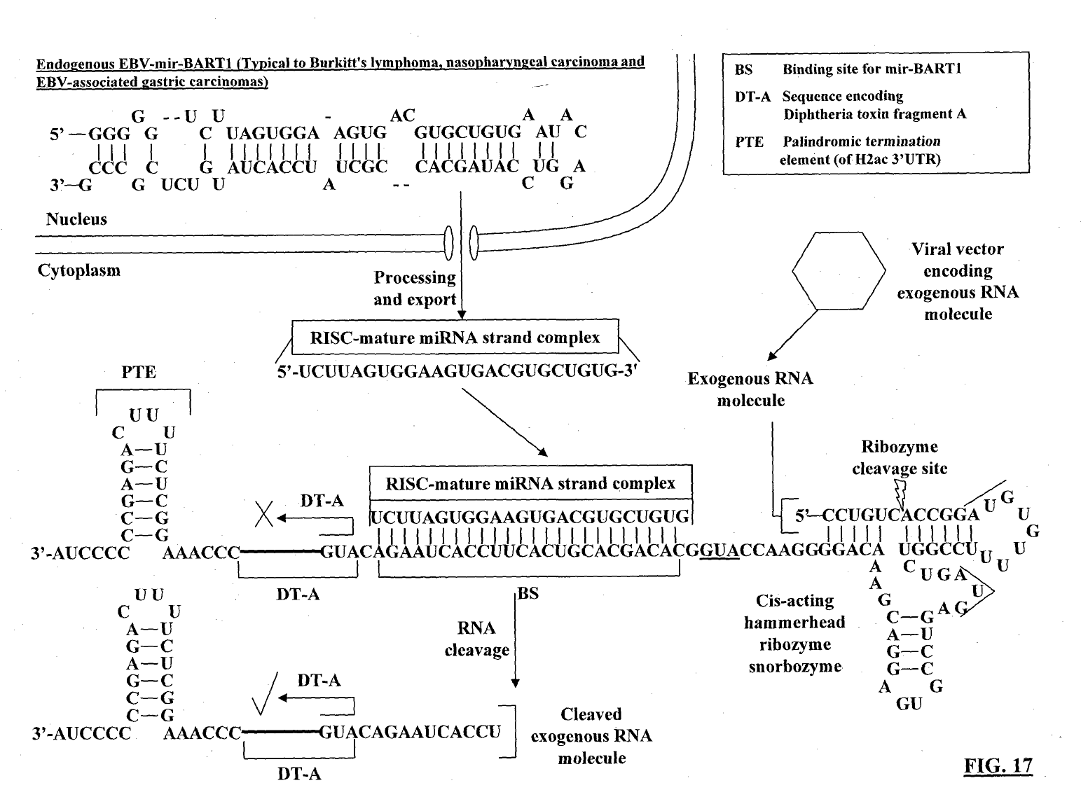

The mature endogenous miRNA strand of EBV-mir-BART1 is: 5′-UCUUAGUGGAAGUGACGUGCUGUG-3′ (SEQ ID NO. 42), the binding site of the exogenous RNA molecule of the example is designed to comprise the sequence: 3′-AGAAUCACCUUCACUGCACGACAC-5′ (SEQ ID NO. 43) that is 100% complementary to the mature endogenous miRNA strand of EBV-mir-BART1. For example, see FIG. 17.

The sequence encoding the exogenous protein of interest is designed to encode the Diphtheria toxin fragment A (DT-A) and is designed to be located downstream from the EBV-mir-BART1 binding site in the exogenous RNA molecule. A single molecule of Diphtheria toxin fragment A introduced into a cell can kill the cell [5] and in mammal cells, the removal of a cap reduces translation of mRNA by 35-50 fold and reduces the functional mRNA half-life only by 1.7-fold [6]. For example, see FIG. 17.

The inhibitory sequence is located upstream from the EBV-mir-BART1 binding site and it is designed to include an initiation codon that is located within the human Kozak consensus sequence: 5′-ACCAUGG-3′ (SEQ ID NO. 25) and is not in the same reading frame with the start codon of DT-A. For example, see FIG. 17.

The exogenous RNA molecule of the example further comprises the very efficient cis-acting hammerhead ribozyme-snorbozyme [15] at the 5′ end for reducing the efficiency of translation of the exogenous RNA molecule before it is cleaved by EBV-mir-BART1. The cis-acting hammerhead ribozyme-snorbozyme also comprises 2 initiation codons however each one of them is not in the same reading frame with the start codon of DT-A. For example, see FIG. 17.

The exogenous RNA molecule of the example also comprises the palindromic termination element (PTE) from the human HIST1H2AC (H2ac) gene 3′UTR (5′-GGCUCUUUUCAGAGCC-3′-SEQ ID NO. 34) downstream from the sequence encoding DT-A. The PTE plays an important role in mRNA processing and stability [7]. Transcripts from HIST1H2AC gene lack poly(A) tails and are still stable thanks to the PTE. For example, see FIG. 17.

In this example, which is illustrated in FIG. 17, the exogenous RNA molecule is transcribed by a viral vector under the control of the strong viral CMV promoter. The sequence of the entire exogenous RNA molecule of this example is set forth as SEQ ID NO. 44.

After the transcription of the exogenous RNA molecule of the example in a target cell, which is introduced with the vector encoding the exogenous RNA molecule, the cis acting ribozyme removes the CAP from the 5′ end for reducing any translation of the exogenous RNA molecule and the palindromic termination element stabilizes the exogenous RNA molecule and protects it from degradation. The out of reading frame initiation codons prevent translation of DT-A, however in the presence of the endogenous EBV-mir-BART1 in the target cell the exogenous RNA molecule of the example is cleaved (the sequence of the cleaved sequence is set forth as SEQ ID NO. 45), and the out of reading frame initiation codons are detached, so that DT-A is translated and expressed in at least one copy of the protein, which is enough to cause cell death. For example, see FIG. 17.]

### Example 3

### Use of the Composition of the Invention to Kill HIV-1 Infected Cells

According to the World Health Organization, in 2006 there were about 39.5 million people with HIV worldwide. According to estimates of the Joint United Nations Program on HIV and AIDS, HIV is set to infect 90 million people in Africa, resulting in a minimum estimate of 18 million orphans. HIV (Human immunodeficiency virus) can lead to the acquired immunodeficiency syndrome (AIDS). Two species of HIV infect humans: HIV-1 and HIV-2. HIV-1 is more virulent, relatively easily transmitted, and is the cause of the majority of HIV infections globally. HIV-2 is less transmittable than HIV-1 and is largely confined to West Africa.

Many viruses, including HIV exhibit a dormant or latent phase, during which little or no protein synthesis is conducted. The viral infection is essentially invisible to the immune system during such phases. Current antiviral treatment regimens are largely ineffective at eliminating cellular reservoirs of latent viruses [1].

Recent genome-wide screens; enabled by computational approaches and high-throughput validation, have discovered 109 microRNA precursors encoded by viruses [13]. Recent studies suggest the role of HIV-1 encoded microRNAs (e.g. miR-N367) in affecting and/or maintaining a latent infection [1, 14 and 19].

HIV-1 transcription is suppressed by nef-expressing miRNA, miR-N367 (SEQ ID NO. 46), in human T cells [19]. The miR-N367 reduces HIV-1 LTR promoter activity through the negative responsive element of the U3 region in the 5′-LTR [19]. Therefore, nef miRNA produced in HIV-1-infected cells may downregulate HIV-1 transcription through both a post-transcriptional pathway and a transcriptional neo-pathway [19].

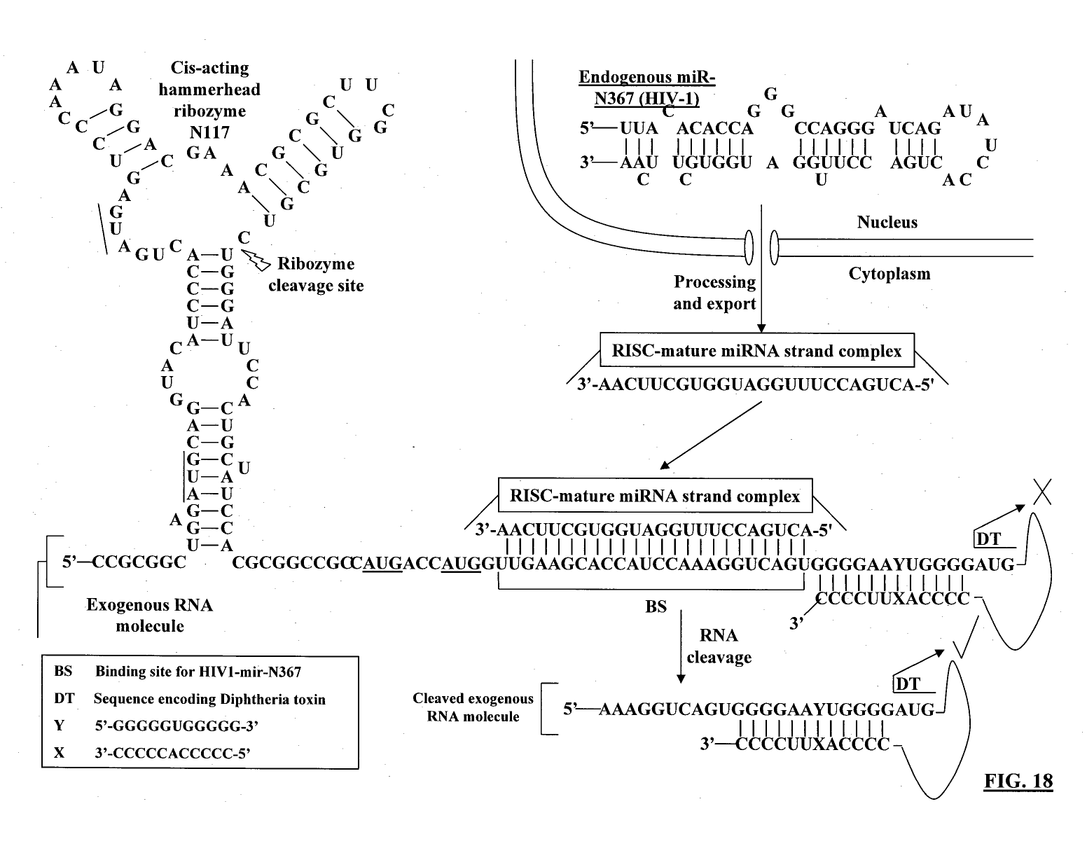

In this example, which is illustrated in FIG. 18, the composition of the invention is designed to kill cells that comprise the endogenous miR-N367 (hiv1-mir-N367) and therefore also comprise HIV-1.

The mature endogenous miRNA strand of miR-N367 is: 5′-ACUGACCUUUGGAUGGUGCUUCAA-3′ (SEQ ID NO. 47), the binding site of the exogenous RNA molecule of the example is designed to comprise the sequence 5′-UUGAAGCACCAUCCAAAGGUCAGU-3′ (SEQ ID NO. 48) that is 100% complementary to the mature miRNA strand of miR-N367. (As illustrated in FIG. 18).

The sequence encoding the exogenous protein of interest is designed to encode Diphtheria toxin (DT) protein and is designed to be located downstream from the miR-N367 binding site in the exogenous RNA molecule. (FIG. 18).

The inhibitory sequence is located upstream from miR-N367 binding site and it is designed to include 2 initiation codons that one of them is located within the human Kozak consensus sequence: 5′-ACCAUGG-3′ (SEQ ID NO. 25) and each of them is not in the same reading frame with the start codon of DT. (FIG. 18).

The exogenous RNA molecule also comprises a nucleotide sequence of 22 nucleotides (SEQ ID NO. 49) downstream from the miR-N367 binding site and upstream from the sequence encoding the DT protein, such that the nucleotide sequence is capable of binding to a sequence of 22 nucleotides (SEQ ID NO. 50) that is located downstream from the sequence encoding the DT, such that the exogenous RNA molecule forms a circular structure that increases the efficiency of translation of DT, particularly when the exogenous RNA molecule is cleaved.

The exogenous RNA molecule also include the very efficient cis-acting hammerhead ribozyme—N117 [16] at the 5′ end for reducing the efficiency of translation of the exogenous RNA molecule before it is cleaved by the endogenous miRNA. The cis-acting hammerhead ribozyme—N117 also comprises 2 initiation codons, none of them is in the same reading frame with the start codon of DT protein. For example, see FIG. 18.

In this example the exogenous RNA molecule is transcribed by a viral vector under the control of the strong viral CMV promoter. The sequence of the entire exogenous RNA molecule of this example is set forth as SEQ ID NO. 51.

After the transcription of the exogenous RNA molecule of the example in a target cell, which is introduced with the vector encoding the exogenous RNA molecule, the cis acting ribozyme removes the CAP from the 5′ end for reducing any translation by the exogenous RNA molecule. The out of reading frame initiation codons prevent translation of DT, however in the presence of the endogenous miR-N367 (or HIV-1) in the cell, the exogenous RNA molecule is cleaved (the sequence of the cleaved sequence is set forth as SEQ ID NO. 52), and the out of reading frame initiation codons are detached from the sequence encoding the DT protein, so that the DT is capable of being expressed. The RNA portion that includes the sequence encoding the DT protein forms a circular structure that increases the translation of the DT protein, for killing the HIV-1 infected cells. For example, see FIG. 18.

The viral vector of the example may also encode transcriptional factors that are capable of enhancing the transcription of HIV1-miR-N367 in HIV-1 infected cell (for example, NF-κB). The viral vector may also encode genes that are capable of preventing new HIV-1 particles production (for example, Rev, which prevents HIV-1 mRNA splicing).

### Example 4

### Use of the Composition of the Invention to Kill Metastatic Breast Cancer Cells

In metastatic breast cancer cells, the expression of miR-10b (SEQ ID NO. 53) is upregulated compared to healthy or nonmetastatic tumourigenic cells [8]. The expression of miR-10b is upregulated by the transcription factor Twist [8]. The target of miR-10b is HOXD10 and reducing in HOXD10 level results in higher level of RHOC and the higher level of RHOC stimulates cancer cell motility [8].

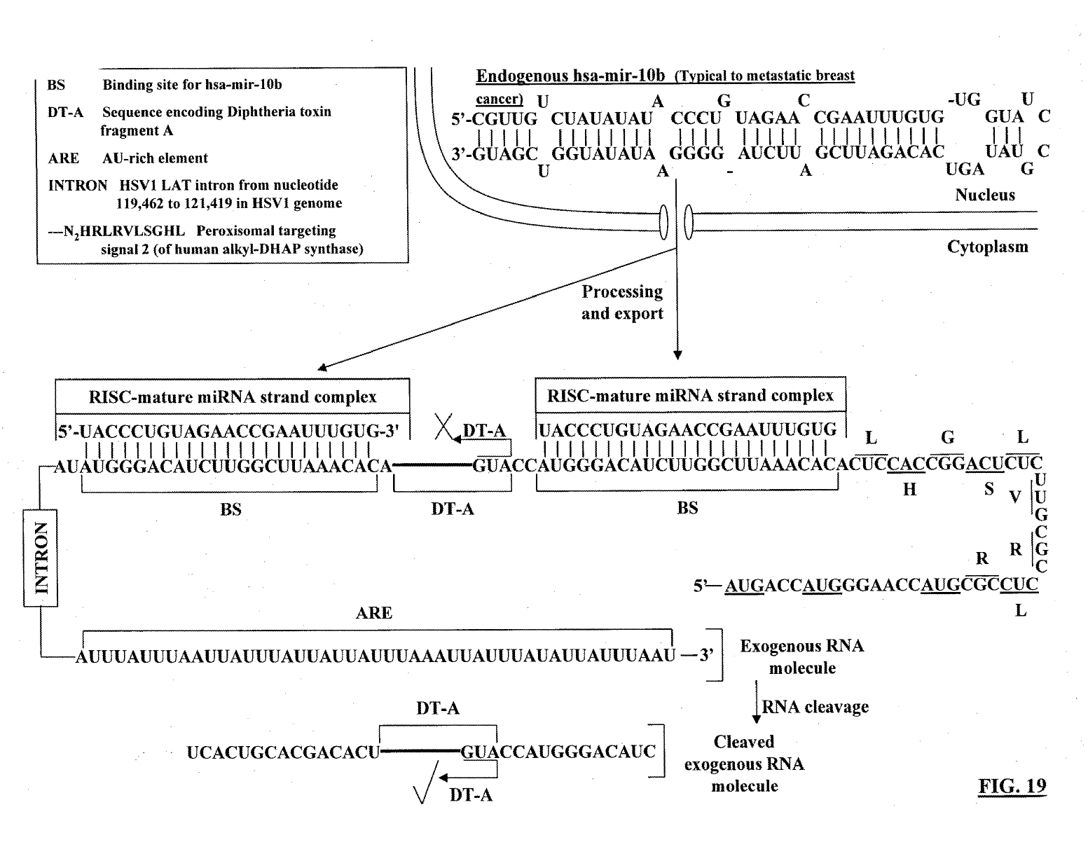

In this example, which is illustrated in FIG. 19, the composition of the invention is designed to kill cells that comprise the endogenous miR-10b, which is typical to metastatic breast cancer cells.

The mature endogenous miRNA strand of miR-10b is: 5′-UACCCUGUAGAACCGAAUUUGUG-3′ (SEQ ID NO. 54), the exogenous RNA molecule of the example is designed to comprise 2 binding sites for miR-10b, such that each one of them comprises the sequence: 5′-CACAAAUUCGGUUCUACAGGGUA-3′ (SEQ ID NO. 55) that is 100% complementary to the mature miRNA strand of miR-10b [31]. (FIG. 19).

The sequence encoding the exogenous protein of interest is designed to encode the Diphtheria toxin fragment A (DT-A) protein and is designed to be located between the 2 binding sites for miR-10b in the exogenous RNA molecule. In mammal cells, a single molecule of Diphtheria toxin fragment A introduced into a cell can kill the cell [5].

The exogenous RNA molecule of the example comprises 2 inhibitory sequences one at the 5′ end and other at the 3′ end.

The inhibitory sequence that is located at the 5′ end of the exogenous RNA molecule is designed to include 3 initiation codons, such that one of them is located within the human Kozak consensus sequence: 5′-ACCAUGG-3′ (SEQ ID NO. 25), and none of them is in the same reading frame with the start codon of the DT-A encoding sequence and such that all the 3 initiation codons are in the same reading frame.

The inhibitory sequence that is located at the 5′ end of the exogenous RNA molecule also include a nucleotide sequence downstream from the 3 initiation codons and upstream from the 2 binding sites for miR-10b, such that the nucleotide sequence is in the same reading frame with the 3 initiation codons and such that the nucleotide sequence encodes for a sorting signal for the subcellular localization that is the Peroxisomal targeting signal 2 of the human alkyl dihydroxyacetonephosphate synthase (H₂N—-RLRVLSGHL—SEQ ID NO. 27) [28]. In mammal cells, proteins that bear a sorting signal for the subcellular localization can be localized to the subcellular localization while they are being translated with their mRNA.

The inhibitory sequence that is located at the 3′ end of the exogenous RNA molecule is designed to include the HSV1 LAT intron downstream from the 2 binding sites for miR-10b, such that the exogenous RNA molecule is a target for nonsense-mediated decay (NMD) that degrades the exogenous RNA molecule that includes an intron downstream from the coding sequence in the exogenous RNA molecule [29].

The inhibitory sequence that is located at the 3′ end of the exogenous RNA molecule also includes an AU-rich element at the 3′ end that stimulates degradation of the exogenous RNA molecule. The AU-rich elements is 47 nucleotides long and it includes the sequences: 5′-AUUUA-3′ (SEQ ID NO. 31) and 5′-UUAUUUA(U/A)(U/A)-3′ (SEQ ID NO. 32) [26].

In this example the exogenous RNA molecule is transcribed by a viral vector under the control of the strong viral CMV promoter. The sequence of the entire exogenous RNA molecule of this example is set forth as SEQ ID NO. 56.

After the transcription of the exogenous RNA molecule of the example in a target cell, which is introduced with the vector encoding the exogenous RNA molecule, the out of reading frame initiation codons prevent translation of DT-A, the Peroxisomal targeting signal 2 sends the erroneous protein and the exogenous RNA molecule to the peroxisome, the intron targets the exogenous RNA molecule to degradation by the nonsense-mediated decay (NMD) and the AU-rich element also stimulates degradation of the exogenous RNA molecule. However in the presence of the endogenous miR-10b in the cell, the exogenous RNA molecule is cleaved (the sequence of the cleaved sequence is set forth as SEQ ID NO. 57), and all the inhibitory sequences are detached, so that DT-A protein is translated and expressed in at least one copy of the protein, which is enough to cause cell death.

### Example 5

### Use of the Composition of the Invention to Kill HSV-1 Infected Cells

Many viruses, including HSV-1 (herpes simplex virus-1) exhibit a dormant or latent phase, during which no protein synthesis is conducted. The viral infection is essentially invisible to the immune system during such phases. Current antiviral treatment regimens are largely ineffective at eliminating cellular reservoirs of latent viruses [1].

The latency-associated transcript (LAT) of herpes simplex virus-1 (HSV-1) is the only viral gene expressed during latent infection in neurons. LAT inhibits apoptosis and maintains latency by promoting the survival of infected neurons. No protein product has been attributed to the LAT gene. Studies suggest that the miRNA-miR-LAT (SEQ ID NO. 58) encoded by the HSV-1 LAT gene confers resistance to apoptosis [17]. miR-LAT is generated from the exon 1 region of the HSV-1 LAT gene and therefore miR-LAT is expressed during latent infection [17].

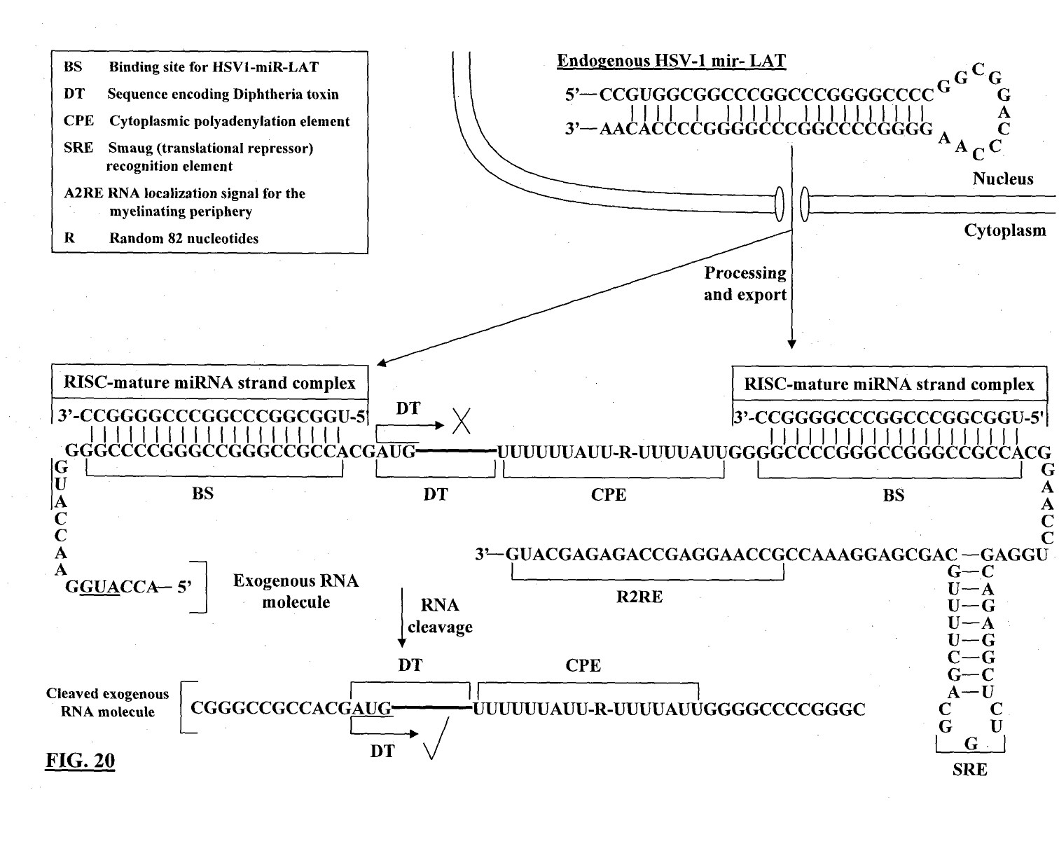

In this example, which is illustrated in FIG. 20, the composition of the invention is designed to kill cells that comprise the endogenous miR-LAT and therefore also comprise HSV-1.

The mature endogenous miRNA strand of miR-LAT is: 5′-UGGCGGCCCGGCCCGGGGCC-3′ (SEQ ID NO. 59), and the exogenous RNA molecule of the example is designed to include 2 binding sites for miR-LAT, such that each one of binding sites include the sequence: 5′-GGCCCCGGGCCGGGCCGCCA-3′ (SEQ ID NO. 60) that is 100% complementary to the mature miRNA strand of miR-LAT [17].

The sequence encoding the exogenous protein of interest is designed to encode the Diphtheria toxin (DT) protein and is designed to be located between the 2 miR-LAT binding sites in the exogenous RNA molecule (FIG. 20).

The exogenous RNA molecule also includes 2 inhibitory sequences, one at the 5′ end and other at the 3′ end.

The inhibitory sequence that is located at the 5′ end of the exogenous RNA molecule is designed to include 2 initiation codons that each one of them is located in the human Kozak consensus sequence: 5′-ACCAUGG-3′ (SEQ ID NO. 25) and none of them is in the same reading frame with the start codon of DT protein. (FIG. 20).

The inhibitory sequence that is located at the 3′ end of the exogenous RNA molecule is designed to comprise the translational repressor smaug recognition elements (SRE): 5′-UGGAGCAGAGGCUCUGGCAGCUUUUGCAGCG-3′ (SEQ ID NO. 28) downstream from the 2 miR-LAT binding sites. Smaug 1 is encoded in human chromosome 14 and is capable of repressing translation of SRE-containing messengers [24, 25]. Murine Smaug 1 is expressed in the brain and is abundant in synaptoneurosomes, a subcellular region where translation is tightly regulated by synaptic stimulation [24].

The inhibitory sequence that is located at the 3′ end of the exogenous RNA molecule also includes an RNA localization signal for myelinating periphery (A2RE—Nuclear Ribonucleoprotein A2 Response Element): 5′-GCCAAGGAGCCAGAGAGCAUG-3′ (SEQ ID NO. 29) at the 3′ end [27]. A2RE is a cis-acting sequence that is located at the 3′-untranslated region of MBP (Myelin basic protein) mRNA and is sufficient and necessary for MBP mRNA transport to the myelinating periphery of oligodendrocytes [27]. The hnRNP (Heterogeneous Nuclear Ribonucleoprotein) A2 binds the A2RE and mediates transport of MBP [27].

The exogenous RNA molecule also includes a cytoplasmic polyadenylation element (CPE) immediately downstream from the sequence encoding the DT protein. The CPE comprises the sequence 5′-UUUUUUAUU-3′ (SEQ ID NO. 38) immediately downstream from the sequence encoding the DT protein and the sequence 5′-UUUUAUU-3′ (SEQ ID NO. 39), 91 nucleotides downstream from the sequence encoding the DT protein [23]. In mammals, CPEB (cytoplasmic polyadenylation element binding protein) is present in the dendritic layer of the hippocampus (the portion of the brain that is responsible for long-term memory) [30]. In the synapto-dendritic compartment of mammalian hippocampal neurons, CPEB appears to stimulate the translation of α-CaMKII mRNA that comprises CPE by polyadenylation-induced translation [30].

In this example, the exogenous RNA molecule is transcribed by a viral vector under the control of the strong viral CMV promoter. The sequence of the entire exogenous RNA molecule of this example is set forth as SEQ ID NO. 61.

After the transcription of the exogenous RNA molecule of the example in a target cell, which is introduced with the vector encoding the exogenous RNA molecule, the out of reading frame initiation codons prevent translation of DT protein, the Smaug1 (translational repressor) binds to the smaug recognition elements (SRE) and inhibits DT protein translation and the hnRNP A2 binds the A2RE and mediates the transport of the exogenous RNA molecule to the myelinating periphery. However in the presence of the endogenous miR-LAT (of HSV-1) in the target cell, the exogenous RNA molecule is cleaved (the sequence of the cleaved sequence is set forth as SEQ ID NO. 62), and the 2 inhibitory sequences are detached, so that the CPEB (cytoplasmic polyadenylation element binding protein) binds the CPE and stimulates the extension of the polyadenine tail in the cleaved exogenous RNA molecule, such that DT is capable of being expressed and consequently kill the cell as well as neighboring cells.

### LIST OF REFERENCES

1. Weinberg M. S and Morris K. V, 2006. Are viral-encoded microRNAs mediating latent HIV-1 infection? DNA Cell Biol 25: 223-231.
2. Velculescu V E et al, 2006. The Consensus Coding Sequences of Human Breast and Colorectal Cancers, Science 314, 5797: 268-274.
3. Lord M J, Jolliffe N A, Marsden C J, et al, 2003, Ricin Mechanisms of Cytotoxicity, Toxicol Rev 22, 1: 53-64.
4. Jeen-Kuan Chen, Chih-Hung Hung, Yen-Chywan Liaw and Jung-Yaw Lin, 1997, Identification of amino acid residues of Abrin-a A chain is essential for catalysis and reassociation with Abrin-a B chain by site-directed mutagenesis, Protein Engineering 10: 827-833.
5. Yamaizumi, M, Mekada, E, Uchida, T. and Okada Y, 1978, One molecule of Diphtheria toxin fragment A introduced into a cell can kill the cell, cell 15, 1: 245-50.
6. Gallie D R, 1991, The cap and poly(A) tail function synergistically to regulate mRNA translational efficiency. Genes Dev 5, 11: 2108-16.
7. Entrez Gene: HIST1H2AC histone cluster 1, H2ac.
8. T. Dalmay, 2008, MicroRNAs and cancer, Journal of Internal Medicine 263, 4: 366-375.
9. Y Zeng, 2006, Principles of micro-RNA production and maturation, Oncogene 25: 6156-6162.
10. B M Engels and G Hutvagner, 2006, Principles and effects of microRNA-mediated post-transcriptional gene regulation, Oncogene 25: 6163-6169.
11. Benjamin Haley & Phillip D Zamore, 2004, Kinetic analysis of the RNAi enzyme complex, Nature Structural & Molecular Biology 11: 599-606.
12. William C S Cho, 2007, OncomiRs: the discovery and progress of microRNAs in cancers. Molecular Cancer 6: 60.
13. Vinod Scaria and Vaibhav Jadhav, 2007, microRNAs in viral oncogenesis, Retrovirology 4: 82.
14. Vinod Scaria, Manoj Hariharan, Beena Pillai, Souvik Maiti, Samir K. Brahmachari, 2007, Host-virus genome interactions: macro roles for microRNAs, Cellular Microbiology 9, 12: 2784-2794.
15. Maurille J. Fournier et al, 1999. A small nucleolar RNA: ribozyme hybrid cleaves a nucleolar RNA target in vivo with near-perfect efficiency, PNAS 96, 12: 6609-6614.
16. Laising Yen, Jennifer Svendsen, Jeng-Shin Lee, John T. Gray, Maxime Magnier, Takashi Baba, Robert J. D'Amato & Richard C. Mulligan, 2004, Exogenous control of mammalian gene expression through modulation of RNA self-cleavage, Nature 431, 471-476.
17. Gupta, J. J. Gartner, P. Sethupathy, A. G. Hatzigeorgiou & N. W. Fraser, 2006, Anti-apoptotic function of a microRNA encoded by the HSV-1 latency-associated transcript, Nature 442, 82-85.
18. http://microrna.sanger.ac.uk/cgi-bin/sequences/miRNA_entry.pl?acc=MI0000267
19. Shinya Omoto and Yoichi R. Fujii, 2005, Regulation of human immunodeficiency virus 1 transcription by nef microRNA, J Gen Virol 86: 751-755.
20. Grey F, Meyers H, White E A, Spector D H, Nelson J, 2007, A Human Cytomegalovirus-Encoded microRNA Regulates Expression of Multiple Viral Genes Involved in Replication. PLoS Pathog 3, 11: e163. doi:10.1371/journal.ppat.0030163.
21. Do Nyun Kim, Hiun-Suk Chae, Sang Taek Oh, Jin-Hyoung Kang, Cho Hyun Park, Won Sang Park, Kenzo Takada, Jae Myun Lee, Won-Keun Lee, and Suk Kyeong Lee, 2007, Expression of Viral MicroRNAs in Epstein-Barr Virus-Associated Gastric Carcinoma, Journal of Virology p. 1033-1036, Vol. 81, No. 2.
22. Chabanon Herve and Ian Mickleburgh, 2004, Zipcodes and postage stamps: mRNA localisation signals and their trans-acting binding proteins, Briefings in Functional Genomics and Proteomics 3:240-256.
23. Wu L, Wells D, Tay J, Mendis D, Abbott M A, Barnitt A, Quinlan E, Heynen A, Fallon J R, Richter J D, 1998, CPEB-mediated cytoplasmic polyadenylation and the regulation of experience-dependent translation of alpha-CaMKII mRNA at synapses, Neuron 21, 5: 936-8.
24. Maria V. Baez and Graciela L. Boccaccio, Career investigator of the Consejo Nacional de Investigaciones Cientificas y Tecnologicas, 2005, Mammalian Smaug Is a Translational Repressor That Forms Cytoplasmic Foci Similar to Stress Granules, J. Biol. Chem. 280, 52: 43131-43140.
25. C A Smibert, J E Wilson, K Kerr, and P M Macdonald, 1996, smaug protein represses translation of unlocalized nanos mRNA in the Drosophila embryo, Genes & Development 10:2600-2609.
26. Carine Barreau, Luc Paillard and H. Beverley Osborne, 2006, AU-rich elements and associated factors: are there unifying principles?, Nucleic Acids Research 33, 22: 7138-7150.
27. Trent P. Munro, Rebecca J. Magee, Grahame J. Kidd, John H. Carson, Elisa Barbarese, Lisa M. Smith, and Ross Smith, 1999, Mutational Analysis of a Heterogeneous Nuclear Ribonucleoprotein A2 Response Element for RNA Trafficking, J Biol Chem, 274, 48: 34389-34395.
28. Suresh subramani, 1998, Components Involved in Peroxisome Import, Biogenesis, Proliferation, Turnover, and Movement, PHYSIOLOGICAL REVIEWS 78: 171-188.
29. Isken O, Maquat L E, 2007, Quality control of eukaryotic mRNA: safeguarding cells from abnormal mRNA function, Genes Dev 21, 15:1833-56.
30. Joel D. Richter, 2001, Think globally, translate locally: What mitotic spindles and neuronal synapses have in common, Proc Natl Acad Sci USA 98, 13: 7069-7071.
31. http://microrna.sanger.ac.uk/cgi-bin/sequences/miRNA_entry.pl?acc=MI0000267
32. Michael S. Wollenberg and Sanford M. Simon, 2004, Signal Sequence Cleavage of Peptidyl-tRNA Prior to Release from the Ribosome and Translocon, J. Biol. Chem. 279: 24919-24922.
33. Dan Frumkin, Adam Wasserstrom, Shalev Itzkovitz, Tomer Stern, Alon Harmelin, Raya Eilam, Gideon Rechavi and Ehud Shapiro, 2008, Cell Lineage Analysis of a Mouse Tumor, Cancer Research 68: 5924-5931.
34. Ugo Moens, 2009, Silencing Viral MicroRNA as a Novel Antiviral Therapy? Journal of Biomedicine and Biotechnology.
35. Zhumur Ghosh, Bibekanand Mallick and Jayprokas Chakrabarti, 2009, Cellular versus viral microRNAs in host-virus interaction. Nucleic Acids Res.
36. Michaelis M, Doerr H W, Cinatl J. Neoplasia, 2009, The story of human cytomegalovirus and cancer: increasing evidence and open questions. Neoplasia Press.
37. Theodore W H, Epstein L, Gaillard W D, Shinnar S, Wainwright M S, Jacobson S, 2008, Human herpes virus 6B: a possible role in epilepsy?. Epilepsia.
38, Elfakess R, Dikstein R. (2008). A translation initiation element specific to mRNAs with very short 5′UTR that also regulates transcription. PloS One. 2008 Aug. 28; 3(8):e3094.

1-42. (canceled)

43. A composition comprising one or more polynucleotides for directing expression of an exogenous protein of interest specifically in a cell expressing a specific endogenous miRNA, said one or more polynucleotides encoding an exogenous RNA molecule, which comprises:
a) a sequence encoding for the exogenous protein of interest;
b) an inhibitory sequence that is capable of inhibiting the expression of the exogenous protein of interest; and
c) a binding site for said specific endogenous miRNA,
whereby only in the presence of said specific endogenous miRNA, the exogenous RNA molecule is cleaved at a cleavage site, thereby releasing the inhibitory sequence from the sequence encoding the exogenous protein of interest such that the exogenous protein of interest is capable of being expressed.

44. The composition of claim 43, wherein said cleavage site is located within said binding site and wherein the cleavage site is located between the inhibitory sequence and the sequence encoding the exogenous protein of interest.

45. The composition of claim 43, wherein said binding site for the specific endogenous miRNA is of sufficient complementarity to a sequence within said specific endogenous miRNA, for said specific endogenous miRNA to direct cleavage of said exogenous RNA molecule at the cleavage site, said specific endogenous miRNA is a cellular microRNA, or a viral microRNA, expressed by a virus selected from the group consisting of a double-stranded DNA virus, a single-stranded DNA virus, a double-stranded RNA virus, a double-stranded RNA virus, a single-stranded (plus-strand) virus, a single-stranded (minus-strand) virus and a retrovirus.

46. The composition of claim 43, wherein said endogenous microRNA is expressed specifically in neoplastic cells.

47. The composition of claim 43, wherein the exogenous protein of interest is a toxin, selected from the group consisting of: Ricin, Ricin A chain, Abrin, Abrin A chain, Diphtheria toxin A chain, alpha toxin, saporin, maize RIP, barley RIP, wheat RIP, corn RIP, rye RIP, flax RIP, Shiga toxin, Shiga-like RIP, momordin, thymidine kinase, pokeweed antiviral protein, gelonin, Pseudomonas exotoxin, Pseudomonas exotoxin A, Escherichia coli cytosine deaminase and modified forms thereof.

48. The composition of claim 43, wherein said inhibitory sequence is located upstream from the cleavage site, and wherein said inhibitory sequence reduces the efficiency of translation of said exogenous protein of interest from said exogenous RNA molecule.

49. The composition of claim 48, wherein said inhibitory sequence comprises a plurality of initiation codons, wherein each of said initiation codons and said sequence encoding exogenous protein of interest are not in the same reading frame, and/or wherein each of said initiation codons is consisting essentially of 5′-AUG-3′, and/or wherein each of said initiation codons is located within a Kozak consensus sequence.

50. The composition of claim 43, wherein said inhibitory sequence is capable of binding to a polypeptide, wherein said polypeptide reduces the efficiency of translation of said exogenous protein of interest from said exogenous RNA molecule, or wherein said inhibitory sequence comprises an RNA localization signal for subcellular localization, an endogenous miRNA binding site, or both.

51. The composition of claim 43, wherein said composition further comprises a polynucleotide sequence encoding a functional RNA that is capable of inhibiting the expression, directly or indirectly, of an endogenous exonuclease.

52. The composition of claim 43, wherein said binding site for the specific endogenous miRNA is a plurality of binding sites for the same or different endogenous miRNAs and wherein said cleavage site is a plurality of cleavage sites.

53. The composition of claim 43, wherein said polynucleotide comprises one or more DNA molecules, one or more RNA molecules or combinations thereof.

54. The composition of claim 43, wherein said exogenous RNA molecule further comprises a stop codon that is located between the initiation codon and the start codon of said sequence encoding protein of interest, wherein said stop codon and said initiation codon are in the same reading frame and wherein said stop codon is selected from the group consisting of: 5′-UAA-3′,5′-UAG-3′ and 5′-UGA-3′.

55. The composition of claim 43, wherein said cell is selected from the group consisting of: human cell, animal cell, cultured cell and plant cell.

56. The composition of claim 43, wherein said composition is introduced into a cell, said cell is present in an organism.

57. A diagnostic kit comprising the composition of claim 43.

58. A pharmaceutical composition comprising the composition of claim 43 and one or more excipients.

59. A method for targeted killing of a target cell, the method comprising introducing into the target cell the composition of claim 43, wherein the target cell comprises the specific endogenous miRNA.

60. A method of treating cancer in a subject in need thereof, the method comprising administering the pharmaceutical composition of claim 58 to said subject, whereby the cancer cells of said subject comprises the specific endogenous miRNA, thereby treating cancer in said subject.

61. A vector comprising a polynucleotide sequence encoding for an exogenous RNA molecule, wherein said exogenous RNA molecule comprises:
a) a sequence encoding for an exogenous protein of interest;
b) an inhibitory sequence that is capable of inhibiting the expression of the exogenous protein of interest; and
c) a binding site for a specific endogenous miRNA.

62. The vector of claim 61, wherein said vector is a viral vector or a non viral vector.

63. The vector of claim 61, wherein said binding site for the specific endogenous miRNA is of sufficient complementarity to a sequence within a specific endogenous miRNA for the specific endogenous miRNA to direct cleavage of said exogenous RNA molecule at the cleavage site, upon introducing the vector into a cell comprising said specific endogenous miRNA.

64. The vector of claim 63, wherein said cleavage site is located within said binding site for the specific endogenous miRNA, and wherein the cleavage site is located between the inhibitory sequence and the sequence encoding the exogenous protein of interest.

65. The vector of claim 61, wherein the specific endogenous miRNA is a cellular microRNA, a viral microRNA, or both.

66. The vector of claim 61, wherein the exogenous protein of interest is a toxin, selected from Ricin, Ricin A chain, Abrin, Abrin A chain, Diphtheria toxin A chain, alpha toxin, saporin, maize RIP, barley RIP, wheat RIP, corn RIP, rye RIP, flax RIP, Shiga toxin, Shiga-like RIP, momordin, thymidine kinase, pokeweed antiviral protein, gelonin, Pseudomonas exotoxin, Pseudomonas exotoxin A, Escherichia coli cytosine deaminase and modified forms thereof.
